(19) United States
(12) Patent Application Publication
(10) Pub. No.: US 2012/0323112 A1
(43) Pub. Date: Dec. 20, 2012

(54) NANOPARTICLES FOR ACCOUSTIC IMAGING, METHODS OF MAKING, AND METHODS OF ACCOUSTIC IMAGING

(75) Inventors: Jesse Jokerst, San Francisco, CA (US); Sanjiv S. Gambhir, Portola Valley, CA (US)

(73) Assignee: The Board of Trustees of the Leland Stanford Junior University, Palo Alto, CA (US)

(21) Appl. No.: 13/495,218

(22) Filed: Jun. 13, 2012

Related U.S. Application Data
(60) Provisional application No. 61/498,087, filed on Jun. 17, 2011.

(51) Int. Cl. A61B 6/00; C12N 5/071; A61B 5/055; A61B 8/13; A61B 6/02

(52) U.S. Cl. 600/420; 600/431; 600/425; 435/325

(57) ABSTRACT

Embodiments of the present disclosure provide for nanoparticles for acoustic imaging, methods of using the nanoparticles, methods of imaging a condition, and the like. Embodiments of the present disclosure include nanoparticles (e.g., silica nanoparticles) that can be used to image, detect, study, monitor, evaluate, and/or screen a sample or subject (e.g., whole-body or a portion thereof).

### CROSS-REFERENCE TO RELATED APPLICATION

This application claims priority to U.S. provisional application entitled “SILICA NANOPARTICLES, METHODS OF MAKING, AND METHODS OF USE,” having Ser. No. 61/498,087, filed on Jun. 17, 2011, which is entirely incorporated herein by reference.

### FEDERAL SPONSORSHIP

This invention was made with Government support under Contract/Grant No. U54 CA119367 and R25-T CA118681, awarded by the National Cancer Institute. The Government has certain rights in this invention.

### BACKGROUND

Imaging monitors the efficacy of cardiac regenerative medicine by reporting the viability, location, and number of implanted stem cells. Strategies utilizing positron emission tomography (PET), magnetic resonance imaging (MRI), and other approaches have been reported. However, these techniques have limitations including temporal resolution, the incorporation of reporter genes, and expensive, bulky equipment. Thus, there is a need to overcome these limitations.

### SUMMARY

Embodiments of the present disclosure provide for nanoparticles for acoustic imaging, methods of using the nanoparticles, methods of imaging a condition, and the like. Embodiments of the present disclosure include nanoparticles (e.g., silica nanoparticles) that can be used to image, detect, study, monitor, evaluate, and/or screen a sample or subject (e.g., whole-body or a portion thereof).

An embodiment of the present disclosure includes a method of imaging cells, among others, that includes: disposing one or more cells in a subject, wherein one or more of the cells includes one or more silica nanoparticles within the cell; imaging the subject and the cells to determine the placement of the cells within the subject; and detecting an acoustic signal, wherein the acoustic signal correlates to the position of the cells within the subject.

An embodiment of the present disclosure includes a method of making cells, among others, that includes: exposing one or more cells to a plurality of silica nanoparticles; and incubating the cells and silica nanoparticles, wherein the silica nanoparticles become disposed within the cells.

An embodiment of the present disclosure includes a method of imaging a target, among others, that includes: exposing a subject to an imaging device, wherein the subject was introduced to a silica nanoparticle, wherein the silica nanoparticle includes a targeting agent having an affinity for a target; and detecting the silica nanoparticles, wherein the location of the silica nanoparticles correlates to the location of the target.

An embodiment of the present disclosure includes a method of imaging a target, among others, that includes: exposing a subject to an imaging device, wherein the subject was introduced to a silica nanoparticle; and detecting the silica nanoparticles, wherein the location of the silica nanoparticles correlates to the location of the target.

An embodiment of the present disclosure includes a method of imaging and treating a subject, among others, that includes: administering to a subject in need of treatment a therapeutically effective amount of an agent, wherein the agent is attached to a silica nanoparticle, wherein the silica nanoparticle includes a targeting agent having an affinity for a target; exposing a subject to an imaging device; and detecting the silica nanoparticles, wherein the location of the silica nanoparticles correlates to the location of the target.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1.1 illustrates the characterization of SiGNR Contrast Agent. TEM images of GNRs (FIG. 1.1(A)) and SiGNRs (FIG. 1.1(C)) were obtained and the materials were studied by absorption spectroscopy at 1:30 dilution of stock solution (˜5 nM) in water. The spectra were normalized relative to the maximum absorbance. A slight red shift noted for the silica-coated agents (FIG. 1.1(B)). FIG. 1.1(D) illustrates the backscatter (B-mode) and PA signals were studied and the addition of the silica coat increased PA signal four-fold. FIG. 1.1(A) and 1.1(C) show some imaging artifacts (white) from heterogeneity in nanoparticle size when imaged in a finite focal depth. This is not indicative of impurity or other species.

FIG. 1.2 illustrates the toxicity and proliferation of SiGNR-labeled MSCs. FIG. 1.2(A) illustrates the capacity of the MTT assay to count cells was confirmed with increasing numbers of plated MSCs (# indicates cytotoxic positive control; 0.25 mg/mL CTAB). FIG. 1.2(B) illustrates the increasing concentrations of SiGNRs show increasing toxicity to 10,000 MSCs after overnight (˜20 hours) incubation with SiGNRs. FIG. 1.2(C) illustrates the incubation time of one concentration (0.07 nM) was further optimized with 3, 6, and 20 hours of incubation. “Ctrl.” in C) indicates no SiGNRs. Incubation at 3 hours at this concentration produced an insignificant (p>0.05) decrease in cell metabolism. FIG. 1.2(D) illustrates a study of the impact SiGNRs have on MSC growth proliferation, MSCs both loaded and unloaded with SiGNRs were serially monitored. There was no significant change to their growth as probed by MTT assay. Both unlabeled and SiGNR-labeled MSCs showed a doubling time of three days. In FIG. 1.2(A)—(C) illustrates the error bars represent the standard deviation of three replicate experiments. Error bars in FIG. 1.2(D) represent standard deviation of six replicate wells.

FIG. 1.3 illustrates the confirmation of SiGNRs Inside MSCs. FIG. 1.3 (A)-(E) illustrate TEM images of MSCs loaded with SiGNRs were collected at increasing magnifications. The dashed, colored inset in FIG. 1.3(A)-(E) correspond to the sequential, higher magnification image in the following panel. FIG. 1.3(C) illustrates the nanorods inside a MSC vesicle. FIG. 1.1 (F) illustrates a portion of the EDS spectra acquired on FIG. 1.3(E) (dashed inset) that confirms the presence of gold. Presence of Cu is from the formvar coated TEM grid and Os is from the OsO₄ stain. The silica coat is not highly visible because the electron density of silica is nearly equivalent to the stained cells. See additional example in FIG. 1.14.

FIG. 1.4 illustrates histology images confirm that the osteogenic and adipogenic differentiation capacity of MSCs is unchanged by presence of SiGNRs. Cells in images on top row are non-induced controls, while the bottom row was cultured in either osteogenic (left) or adipogenic (right) media. The experiments presented in FIG. 1.4(B), (F), (D), and (H) were performed on cells loaded with SiGNRs before plating. FIG. 1.4(A), (E), (C), and (G) illustrate the unloaded control cells. Both loaded and unloaded cells show increased mineral deposition as determined by Alizarin Red S staining in the osteogenic experiments (red color; FIG. 1.4(E), (F)). Differentiation into adipocytes is similarly unaffected by the presence of the nanoparticles. Black arrows in FIG. 1.4(G) and (H) highlight lipid vacuoles stained by Oil Red O. Importantly, not only is differentiation capacity retained, but the presence of SiGNRs does not induce unintended differentiation (FIG. 1.3(B) and (D)). See FIG. 1.17 for white light photographs of the cell culture plates.

FIG. 1.5 illustrates secretome analysis of labeled cells. The change in secretome cytokine expression levels is shown for 26 different proteins. Cell culture media from SiGNR-loaded MSCs and control MSCs was analyzed for these proteins. The concentration of protein in SiGNR-loaded cells was divided by the concentration in controls cells to produce the metric above. Except for IL-6, no protein had a concentration that changed more than 200% (1-fold change). Black bars indicate a statistically significant (p<0.05) change in expression; green bars indicate a p-value above 0.05. Please see Table 1, FIG. 1.17 for actual values and additional statistical content.

FIG. 1.6 illustrates in vivo positive and negative controls; labeled MSC injection. This figure presents both B-mode (grey scale) and PA (red) images of the intra-muscular injection of a positive control (0.7 nM SiGNRs; left), negative control (0 nM SiGNRs (no cells); middle), and 800,000 SiGNR-labeled MSCs (right) all in 50% matrigel/PBS into hind limb muscle of an athymic mouse. Imaging sequence is as follows: pre-injection (FIG. 1.6(A), (B), (C)); needle insertion and position (FIG. 1.6(D), (E), (F)); post-injection (FIG. 1.6(G), (H), (I)); needle removal and final imaging (FIG. 1.6(J), (K), (L)), and contrast enhancement to illustrate increased signal (FIG. 1.6(M), (N), (O)). Pixels increased relative to pre-injection image are coded yellow. Note significant signal increase in FIG. 1.6(M) and (O) at injection site relative to (FIG. 1.6(A) and (C)) (dashed circles highlight injection site). Also, note low signal in negative control FIG. 1.6(N). Scale bar in M and intensity scale in L/O applies to all images. The “b” in all panels indicates bone and the red dashed circle in FIG. 1.6(J, K, L) indicates that the injection bolus can also be seen with B-mode ultrasound.

FIG. 1.7 illustrates the validation of imaging data. A) Spectral analysis of tissue and 800,000 MSCs after i.m. injection. Also shown in green is the normalized spectral analysis of the MSCs in vivo. A broad increase in PA signal is seen, which may be due to aggregation and resonance coupling of the contrast (see 1.7E and FIG. 1.14). This normalized spectrum is more red-shifted versus SiGNRs imaged in a phantom (FIG. 1.11D). That experiment suggested an intensity maximum near the absorbance peak shown in FIG. 1.1. B) The average signal for decreasing numbers of cells and as well as the negative control indicate that the calculated estimate for limit of detection is 90,000 cells. 100,000 cells were easily imaged above background injection. Error bars represent the standard error of the background-corrected signal for each group of mice. C) H&E staining of muscle tissue (right) with adjacent MSCs (left). D) The fluorescence of a cell tracking dye (green) from an adjacent section illustrates higher signal for MSCs than muscle tissue. E) Higher magnification of MSCs shows SiGNRs (black) inside the cells (black arrows).

FIG. 1.8 illustrates a block diagram of photoacoustic imaging system. The LAZR photoacoustic imaging system uses a fiber-coupled tunable optical parametric oscillator (OPO) laser (680 nm to 950 nm, 10 Hz pulse repetition frequency) integrated to a 256 element linear array ultrasound transducer with a center frequency of 21 MHz. The fiber coupled laser beam is split into two parallel beams on either side of the transducer. The PC contains control sequence, data acquisition board, and a monitor. The control sequence triggers the laser source and the ultrasound transducer such that multiple ultrasound images can be acquired in real time in between two photoacoustic frames due to the low repetition rate of the laser. The monitor can then in real time display both ultrasound and photoacoustic B-mode images reconstructed using the software. The transducer can be scanned in one direction to acquire a three-dimensional (3D) data. Using a beam sampler and a photo detector, photoacoustic images are corrected for pulse to pulse variations in the laser intensity. Please also see FIG. 1.10.

FIG. 1.9 illustrates photographs of photoacoustic imaging system. A) The imaging system (LAZR; Visualsonics) consists of laser excitation source housed in a separate cart coupled to light-tight imaging chamber. B) The animal subject is immobilized on a heated imaging bed with built-in physiological monitoring. Signal is collected by a transducer and processed with a dedicated PC coupled to the imaging chamber.

FIG. 1.10 illustrates the optimization of imaging conditions for SiGNRs. A 0.7 nM inclusion of SiGNRs was imaged under increasing gain (A), power (B), and dynamic range (C). The signal of the inclusion (open diamond) and signal-to-background (black square) were plotted. Note that some background values were zero giving a non-real value for S/B. These were not plotted. Error bars represent the standard deviation of the three replicates measurements of the same inclusion. Here, gain means hardware gain. The analogue signal (in voltage) is amplified when gain is increased. This is before the analogue signal is digitalized and displayed on the screen. Dynamic range means the dynamic range of the digitalized signal. A high dynamic range means that a larger range of digitalized signal amplitudes is displayed (which generally results in a more faded/wash-out image). On the other hand, if a low dynamic range is set, that means the display offers a smaller number of digitalized signal amplitudes (which generally results in a high-contrast image). Power (%) indicates laser power, which is the transmit power of the OPO laser. This is adjusted by modulating the Q-switch delay. For 100% transmit power, the Q-switch delay is optimized for maximum laser energy output. At 50%, the Q-delay is increased until it reaches 50% laser of max laser energy output. Multi-spectral imaging of the contrast agents in an agarose phantom are presented in panels D and E. In D, the PA signal as a function of wavelength are shown for GNRs and SiGNRs as well as the endogenous, low level PA signal of the agarose. Values were normalized using the PA signal of the agarose to create E. Both GNRs and SiGNRs show highest PA signal from 680 nm to 780 nm, which corresponds to their absorbance spectra in FIG. 1.1. The enhancement due to silica coating is shown as the green curve in E and ranges from 3.7- to 1.3-fold. In E, GNR and SiGNR curves correspond to the left axis; the enhancement coefficient corresponds to the right.

FIG. 1.11 illustrates the spatial resolution of imaging system. A) A test pattern with variously spaced lines (each line was 200 μm wide, ¼ point) was printed on a piece of transparency film and immobilized in the scanner and imaged as described in FIG. 1.11. B) 2.03 mm spacing in between lines; C) 1.27 mm spacing; D) 340 μm; and E) 58 μm. When lines profiles of D and E were studied, it is clear that 340 μm spacing can be resolved, but 58 μm spacing cannot be resolved. Bar in A is 3 mm and bar for B-E is 3 mm. Photomicrographs of G) stage micrometer, H) the 340 μm separations, and I) the 58 μm separations. G-H were at all with same 10× objective. In B-mode the axial resolution is 75 μm and the lateral resolution is 165 μm. The temporal resolution is tunable from 7 to 300 frames per second.

FIG. 1.12 illustrates the limit of detection for SiGNRs. A) Decreasing concentrations of SiGNRs from 0.7 to 0 nM were immobilized an agarose phantom and imaged as described above. B) Image processing and regression analysis indicates a LOD of 0.03 nM (R²>0.99). C) SiGNRs were mixed 1:1 with matrigel implanted subcutaneously into nu/nu mice and imaged similarly to the phantom. Dashed ovals indicate location of SiGNR bolus. Error bars in B and D represent the standard deviation of three replicate measurements. The LOD in vivo is 0.05 nM and is linear at R²=0.97.

FIG. 1.13 illustrates the confirmation of SiGNRs inside MSCs. A-D) TEM images of MSCs loaded with SiGNRs were collected at increasing magnifications. The dashed, colored inset in A-D corresponds to the sequential, higher magnification image in the following panel. The black arrow in D highlights aggregated SiGNRs that allow absorption beyond the 676 nm normal resonance ex vivo.

FIG. 1.14 illustrates the MSC imaging via SiGNRs. A) 50,000 SiGNR-labeled MSCs (left), 50,000 GNR-labeled MSCs, and 50,000 label free MSCs are shown in B-mode (A), cross section (B) and as a maximum intensity projection (C). Yellow line in C corresponds to cross section shown in B. Note increased signal due to presence of nanorods in MSCs and also notice dramatic increase in PA in SiGNR-MSCs than GNR-MSCs (7.6-fold). D) Decreasing numbers of SiGNR-labeled MSCs were immobilized in an agar phantom and imaged to give a limit of detection of 5,000. Error bars represent the standard deviation of five replicate measurements and in each experiment cells were dissolved in 15 μL of 50% water/50% 1 mg/mL agarose.

FIG. 1.15 illustrates the inductively coupled plasma analysis of gold standards and cells. A) Nanorods) and cells (B) were digested and analyzed for gold content. The MSCs with SiGNRs were significantly (p<0.01) elevated above non-silica GNRs as well as control cells (no contrast agent). This standard curve allowed the calculation of 102,000±1,000 SiGNRs/cell. In A) all samples were dissolved in 5 mL total volume. The highest calibration datum (1.45×10¹¹) corresponds to 15.4 mg/L or 15.4 ppm gold.

FIG. 1.16 illustrates the photographs of differentiation experiments. Cells in images on top row are non-induced controls, while the bottom row was cultured in either osteogenic (left) or adipogenic (right) media. The experiments presented in panels B, F, D, and H were performed on cells loaded with SiGNRs before plating. Panels A, E, C, and G are unloaded control cells. Both loaded and unloaded cells show increased mineral deposition as determined by Alizarin Red S staining in the osteogenic experiments (E, F). Differentiation into adipocytes is similarly unaffected by the presence of the nanoparticles as shown by increased red staining of the lipid vacuoles by Oil Red O. Importantly, not only is differentiation capacity retained, but the presence of SiGNRs does not induced unintended differentiation (B and D). See FIG. 1.4 for detailed microscopy.

FIG. 1.17 illustrates Table 1 showing the cytokine analysis of MSC secretome. 31 different proteins were measured in the cell culture media of MSCs (column “Normal”) and SiGNR-MSCs (column “SiGNR”). Culture medium with no MSCs was also analyzed (column “Media”). The limit of detection (LOD) of the assay is presented as well as the coefficient of variation (CV) in the assay at the given concentration regime. Values of p were calculated to determine whether the increase was statistically significant using p=0.05 as the discriminating value.

FIG. 1.18 illustrates the imaging MSCs in vivo and MSC detection limits. Decreasing numbers of SiGNR-labeled MSCs (in 80 μL 50% matrigel) were injected into the hind limb muscle of athymic mice (n=3 at each cell number) as well as a vehicle control (see FIGS. 6N and 7B): A) 800,000 MSCs, B), 300,000 MSCs, and C) 100,000 MSCs. Post processing images present endogenous signal in red; green/yellow indicates pixels that were increased due to injection of SiGNR-labeled MSCs. The b indicates bone. Panels D-I are serial imaging of one bolus of 800,000 cells injected in a separate experiment. Panel D is pre-injection; E is immediately post-injection; F, G, H, and I are 1, 3, 4, and 7 days post injection, respectively. Clear discrimination between MSCs and surrounding tissue could be imaged up to four days despite challenges in exact animal repositioning.

FIG. 1.19 illustrates the ex vivo analysis of the muscle tissue treated with SiGNR-loaded MSCs. Panel A is a white light image of the treated tissue with higher magnification in B. Blue dashed circle in B indicates the bolus of cells loaded with SiGNRs as well as a cell tracker fluorophore (SP-DiOC18(3)). The sample was placed in a fluorescence imaging system with green fluorescent protein filter cubes appropriate for the cell tracking dye and imaged. Fluorescence signal on the sample correlates to the location of the MSCs. Scale bar in A and C is 10 mm and scale bar in B is 4 mm.

FIG. 2.1 illustrates SiNPs are a multimodal contrast agent for stem cell therapy. FIG. 2.1(A) Cardiac stem cell therapy utilizes mesenchymal stem cells loaded with silica nanoparticles. The silica nanoparticles are constructed of a silica (Si0₂) framework (grey) that stabilizes Gd³⁺ (red) and FITC fluorophores (green) with an impedance mismatch that backscatters ultrasound (black waves). FIG. 2.1(B) A FITC reporter (green) is linked to the SiO₂ matrix via a silane terminal group (grey). FIG. 2.1(C) TEM image of SiNPs show spherical particles with a diameter of ˜300 nm. (Scale bar=250 nm).

FIG. 2.2 illustrates imaging MSCs with SiNPs via transmission electron microscopy. MSCs were fixed, stained, sectioned, and placed on copper grids for TEM imaging. FIG. 2.2(A) 440× magnification unloaded cell. FIG. 2.2(B) SiNP-loaded cell at 440× magnification with red arrows highlighting dark spots (SiNPs) distributed throughout the cell. FIG. 2.2(D) 4000× magnification image of SiNPs. The dark area was confirmed to be SiNPs via EDS (FIG. 2.2(E)), which showed the characteristic silicon peak at 1740 eV. White areas in B indicate ripping of the epoxy resin support matrix that occurs due to presence of SiNPs during microtoming (green arrows). Inset in FIG. 2.2(E) presents distribution of SiNP location throughout 45 cells (error bars represents the standard error). The increased echogenicity of MSCs is illustrated in FIG. 2.2(G) and FIG. 2.2(H). FIG. 2.2(G) is a representative 40 MHz B-mode US image of 50,000 MSCs without SiNPs and imaged in an agarose phantom. FIG. 2.2(H) is a similar image containing 50,000 SiNP-loaded MSCs. Green outline highlights the cell pellet in agarose phantom. Signal is 3-fold higher in the labeled cells. FIG. 2.2(C) presents the signal of decreasing numbers of cells in the same phantom. The LOD was 650 cells at 40 MHz. In FIG. 2.2©, error bars represent the standard deviation of five replicate measurements at each concentration. FIG. 2.2(F) Decreasing amounts of cells were also analyzed by T1 SE imaging. From the linear portion of the graph, a LOD of 340,000 cells was calculated.

FIG. 2.3 illustrates the influence of SiNP on MSC metabolism, proliferation, and secretome. FIG. 2.3(A) Triplicate wells of 20,000 cells were incubated with increasing amounts of SiNPs or media only and their metabolic activity was determined by MTT Assay. No statistically significant decrease in metabolic activity between the control cells and loaded cells was observed. FIG. 2.3(B) Growth rates of loaded (0.5 mg/mL for 6 hours; 6540±620 SiNPs/MSC) and unloaded cells are also similar and show a doubling time of ˜48 hours for both loaded and unloaded MSCs. FIG. 2.3(C) reports the change in secretome cytokine expression levels for 26 different proteins. Cell culture media from SiNP-loaded MSCs and control MSCs were analyzed for these proteins. The concentration of protein in SiNP-loaded cells was divided by the concentration in controls cells to produce the metric above. Black bars indicate a statistically significant (p<0.05) change in expression; green bars indicate a p-value above 0.05. Please see Table 1, FIG. 2.27, for actual values and additional statistical content.

FIG. 2.4 illustrates the pluripotency of MSCs is retained after labeling with SINPs. Cells in images on top row are osteogenic controls, middle row is adipogenic, and bottom row is chondrogenic controls cultured in either control media (left) or differentiation media (right). The experiments presented in FIGS. 2.4B, 2.4F, 2.4J, 2.4D, 2.4H, and 2.4L were performed on cells loaded with SiNPs before plating. FIGS. 2.4A, 2.4E, 2.4I, 2.4C, 2.4G, 2.4K, and 2.4L show unloaded control cells. Both loaded and unloaded cells show increased mineral deposition as determined by Alizarin Red S staining in the osteogenic experiments (red color; FIGS. 2.4C, 2.4D). Differentiation into adipocytes is similarly unaffected by the presence of the nanoparticles. Chondrogeneis is indicated by Alcian blue staining in FIG. 2.4K and 2.4L and is more prevalent than cells grown in control media. Black arrows in FIG. 2.4G and 2.4H highlight lipid vacuoles stained by Oil Red O. Importantly, not only is pluripotency retained, but the presence of SiNPs does not induced unintended differentiation (FIGS. 2.4B, 2.4F, and 2.4J).

FIG. 2.5 illustrates US imaging of MSCs after intracardiac implantation. Short axis views of the left ventricle of nu/nu mice are shown in this figure. Left-most panels are after insertion of a needle catheter, middle panel present an image after injection of payload, and right-most panels are image enhanced version of the post injection image after background subtraction and image analysis. The red arrow represents the bevel of the needle catheter. LV=left ventricle. Three experiments are shown including the vehicle carrier (top), 6 mg of SiNPs (middle), and 1,000,000 SiNP-loaded MSCs (bottom). All scale bars are 2 mm.

FIG. 2.6 illustrates MR imaging of MSCs after US-guided intracardiac delivery. TI SE axial sections of the chest cavity of nu/nu mice are shown in this figure. FIGS. 2.6A and 2.6C are representative pre-injection images for the vehicle control and MSC experiments, respectively. FIG. 2.6D is post injection imaging of 1.5×10⁶ MSC cells with enhanced contrast clearly seen on the left ventricular wall. FIG. 2.6C shows no increased contrast as a result of the matrigel/PBS injection. In all images, LV=the center of the left ventricle; Lu=Lung; Sh=left shoulder; and LD=latissimus dorsi muscle. Red arrow indicates injection site before and after implant. The intensity scale bar in FIG. 2.6A applies to all images. FIG. 2.6E) The MRI and US signal change as function of the number of injected cells. The response was linear for both modalities above R²=0.97. F) Animals injected (on day 0; red datum) with 1,000,000 MSCs were monitored sequentially. The signal of the cell bolus remains elevated above baseline (red datum) in a statistically significant way (p<0.05) for 13 days post injection. Error bars represent the standard error for the three animals in each sample population. (See Methods for explanation of the ordinates.)

FIG. 2.7 illustrates the histology validation of in vivo imaging. We used histology and immunofluoresence to image cardiac tissue after SCT. FIGS. 2.7A-C are 2.7H&E stains of cardiac tissue under increasing magnifications. This sample was explanted one day after treatment with 500,000 MSCs in the left ventricle wall. Clear discrimination is seen between the pinker native tissue and dark purple MSCs. The dashed box illustrates the area subjected to increased magnification in the followed panel. FIG. 2.7D) H&E illustrates LV and heart wall explanted ten days after SCT. Red box indicates treated area and is magnified in FIG. 2.7E. Blue box is untreated area (FIG. 2.7F). FIG. 2.7G and 2.7H are immunofluorescence from an adjacent slice from the same tissue sample. Red signal in FIGS. 2.7D and 2.7E indicates troponin immunofluorescence and green indicates MSCs from the embedded fluorescence of the SiNPs. Treated area shows presence of MSCs while untreated area has only troponin.

FIG. 2.8 illustrates the growth curve and spectral behavior of SiNPs. FIG. 2.8A illustrates the size of SiNPs was measured periodically during the synthesis; the maximum size is reached at approximately 2 hours with no further growth seen in overnight incubation. FIG. 2.8B illustrates a UV-Vis absorbance curve of 0.01 mg/mL SiNPs in water. Red arrow indicates FITC absorption. FIG. 2.8C illustrates excitation (solid) and emission (dashed) spectra of SiNPs (1 μg/mL) and stock FITC (1 μM). Spectra are normalized to their maximum. RFU=relative fluorescence intensity. The US signals for different sized SiNPs in an agarose phantom are shown from imaging at 40 MHz (FIG. 2.8D) and 16 (MHz FIG. 2.8E). Error bars represent the standard deviation of five measurements.

FIG. 2.9 illustrates SiNP characterization. FIG. 2.9A illustrates that the SiNP size was determined via TEM images with a mode size of 300 nm. 83% of all NPs were between 200-500 nm. FIG. 2.9B illustrates a histogram of intensity-weighted DLS shows the bias of that technique to larger particles with a Z-average size of 600 nm (0.1 mg/mL in water).

FIG. 2.10 illustrates optimization of US imaging parameters for SiNPs. Imaging conditions were optimized for (FIG. 2.10A) frequency, (FIG. 2.10B) power, (FIG. 2.10C) gain, and (FIG. 2.10D) dynamic range. A 2.5 mg/mL sample of SiNPs was placed in an agarose phantom and imaged at varying input ultrasound frequencies. Signal was defined as B-mode contrast generated from the SiNP inclusion and background was defined as the US contrast generated from the agarose gel surrounding the inclusion. Signal to background ratio (S/B) was calculated as a descriptor of contrast. Error bars represent the standard deviation of five different fields of view. Default settings for this study include 100% power, transducer gain of 20 dB, dynamic range of 60 dB, 40 MHz frequency, and display map G1.

FIG. 2.11 illustrates the concentration-dependent US contrast and ex vivo limit of detection. B-mode ultrasound images of SiNPs in 1% agarose phantom at the following concentrations: FIG. 2.11A, 4.0 mg/mL; FIG. 2.11B, 2.0 mg/mL; FIG. 2.11C, 1.25 mg/mL; FIG. 2.11D, 0.5 mg/mL; FIG. 2.11E, 0.05 mg/mL. FIG. 2.11F is a water control (0 mg/mL SiNPs). Red scale bar in FIG. 2.11A is 2 mm and applies to all images in FIG. 2.11. Intensity scale in A applies to all panels.

FIG. 2.12 illustrates the performance of SiNPs as US contrast ex vivo. FIG. 2.12A illustrates the B-mode ultrasound intensity of SiNPs plotted as a function of concentration shows a linear dynamic range of 0-5 mg/mL at 40 MHz and 0-2.5 mg/mL at 16 MHz. FIG. 2.12B shows lower concentrations and illustrates the LOD of 11.5 and 100.5 μg/mL for 40 and 16 MHz, respectively. Error bars represent standard deviation of five replicate measurements and both are linear at R²>0.99.

FIG. 2.13 illustrates the MRI LOD and T1-Relaxivity of SiNPs. FIG. 2.13A illustrates the decreasing concentrations of SiNPs in water were evaluated ex vivo. (Concentrations in inset: 1-10 mg/mL; 2-5 mg/mL; 3-2.5 mg/mL; 4-1.25 mg/mL; 5-0.625 mg/mL; red box is diluent control) The LOD is 0.03 mg/mL SiNPs. Error bars represent the standard deviation of the three replicate samples. FIG. 2.13B illustrates the T1 of decreasing concentrations of SiNPs from 2.0 to 0.1 mg/mL was measured in a 1T permanent magnet. The inverse of T1 as a function of concentration indicates a T1 relaxivity value of 6×10⁶/(mM s).

FIG. 2.14 illustrates Gd³⁺ loading and stability analyses of SiNPs via ICP. FIG. 2.13A illustrates increasing amounts of SiNPs were analyzed via ICP-AES, with a linear relationship between volume of SiNPs and moles of Gd³⁺ (R²=0.9999). From this, an average of 1.47×10⁶ Gd³⁺ ions per SiNP were calculated. Error bars represent the standard deviation of triplicate samples. FIG. 2.13B illustrates the stability of the SiNP in presence of diluents and murine serum. Less than 5% of the Gd³⁺ dissociated from the SiNP even in serum at 24 hours at body temperature. Error bars represent the standard deviation of triplicate samples.

FIG. 2.15 illustrates the optimization and characterization of SiNP cell loading. FIG. 2.13A), Loading MSCs with increasing amounts of SiNPs indicated that optimal signal (as determined by peak FC signal) was achieved with 0.5 mg/mL SiNPs. FIG. 2.13B), The length of incubation time was similarly optimized and indicated that 6 hours was sufficient. FIG. 2.13(C), The stability of cell loading was probed by serially analyzing cells post loading (circles). Intensity reached a ½max value at approximately two days post loading. Error bars represent the coefficient of variation of the FC histograms for the labeled cells.

FIG. 2.16 illustrates visualizing MSC loading of SiNPs with fluorescence microscopy. Unlabeled MSCs in FIG. 2.16A show little autofluorescence, while labeled MSCs in FIG. 2.16B show green fluorescence from SiNPs via a 10× objective and standard green fluorescent protein optical filters. Green channel images thresholded above 777 on 12 bit images via ImageJ and brightfield images employ 10 ms of exposure time. Scale bar in FIG. 2.16A and FIG. 2.16B is 100 μm. Panels C-E are confocal images under brightfield illumination in FIG. 2.16C, confocal fluorescence through the cells medial slice in FIG. 2.16D, and merged image in FIG. 2.16E. Confocal (Z-axis) scanning indicated that SiNPs were present not only on the cell exterior, but also inside the cell.

FIG. 2.17 illustrates TEM of SiNPs inside MSCs. 500 nm sections of fixed MSCs were imaged with TEM at various magnifications. Sections are coded by color. FIG. 2.17A illustrates 440× magnification shows MSC with dark areas indicating SiNP. FIGS. 2.17B and 2.17C are at 2100× and show both small SiNPs (yellow arrows) and larger clusters of SiNPs (magenta arrows). FIG. 2.17D offers an additional example at 1600×. Scale bar in FIG. 2.17A is 5000 nm; FIG. 2.17B and FIG. 2.17C is 1000 nm; FIG. 2.17D is 2000 nm.

FIG. 2.18 illustrates a TEM of SiNPs Inside MSCs at increasing magnification. 500 nm sections of fixed MSCs were imaged with TEM at various magnifications. FIGS. 2.18A-C on far left are at 440× magnification. Subset i is at 830×, subset ii is at 1100×, and subset iii is at 2100× (except Aiii at 1600×).

FIG. 2.19 illustrates STEM and EDS mapping of SiNPs inside MSCs. FIGS. 2.19A and 2.19B are STEM images of MSCs including SiNPs (black spots). FIG. 2.19B is magnification of area indicated in FIG. 2.19A. FIG. 2.19C shows a representative positive (red) and negative (black) spectra (with the characteristic silica K electrons at 1700 eV) corresponding to an area with (positive) and without (negative) SiNPs. EDS mapping was done in the areas coded by red and blue boxes, which correspond to FIG. 2.19D and FIG. 2.19E, respectively. FIG. 2.19D and 2.19E show EDS maps of the highlighted areas with the highest red intensity corresponding to most silica. Panel D shows one large (˜500 nm) SiNP and the blue panel (FIG. 2.19E) shows a collection of smaller SiNPs.

FIG. 2.20 illustrates cell-based assays. FIG. 2.20A illustrates the MTT assay was used to count increasing numbers of cells, which confirmed its suitability for cell proliferation assays (FIG. 2.20, main text). Error bars are the standard deviation of four replicate wells and the experiment indicated by asterisk was a positive control that included a cytotoxic agent. FIG. 2.20B illustrates the florescence intensity of the saline wash after cell loading was measured and shows no further decrease after wash 3. Error bars represent the standard deviation of three replicate measurements.

FIG. 2.21 illustrates the cell morphology. MSCs loaded with SiNPs (left) and native cells (right) were studied by light microscopy daily for seven days. Cell morphology is similar in both sets of digital photomicrographs. Images in FIGS. 2.21 A and 2.21B were taken on day 1, FIG. 2.21C and 2.21D are day 3, and FIG. 2.21E and 2.21F day 7.

FIG. 2.22 illustrates a representative T1 weighted image of a phantom with PBS diluent as well as increasing numbers of SiNP-labeled MSCs. Values above section in the phantom indicate the number of MSCs (K=1000; M=million). The * indicates that the 950,000 in that well are unloaded with SiNPs. The presence of the contrast agent increases T1 signal by a factor of 2 (labeled versus unlabeled cells). See FIG. 2.2F.

FIG. 2.23 illustrates the compatibility of SiNP-based contrast with clinical scanners. 40,000 SiNP-loaded MSCs were implanted in an agarose phantom and imaged with 40 MHz (FIG. 2.23A) and 16 MHz (FIG. 2.23B) via the Vevo pre-clinical scanner as well as with a clinical Philips iU22 scanner with 17-5 MHz (FIG. 2.23C) and 12-5 MHz (FIG. 2.23D) transducers. FIG. 2.23E shows replicate measurements as well as the agarose vehicle (diluent). All scale bars are 2 mm and error bars represent standard deviation.

FIG. 2.24 illustrates the impact of SiNP Fraction on US Intensity. FIGS. 2.24A-C are TEM images of a freshly mixed batch of SiNPs showing a distribution of sizes (scale bar=500 nm). The batch was allowed to settle overnight. Then the supernatant was collected and imaged (FIG. 2.24B) showing a more monodisperse collection of SiNPs. The sediment was also imaged and showed larger particles above 1 μm in size. We also measured the US contrast of the three fractions with FIGS. 2.24D-F corresponding to the particle fractions above. Indeed, most of the US signal is generated by the larger aggregates. However, when both the sediment and the supernatant is incubated with cells, the US signal is the same and nearly 7 times higher than unlabeled cells. Error bars represent the standard deviation of five FOVs. Scale bars in FIGS. 2.24A-C are 500 nm. D-F scale bars are 2 mm.

FIG. 2.25 illustrates example Mis-Injection of MSC Detected via US Imaging. In this example, we intended to inject 500,000 SiNP-labeled MSCs (green) into the wall of the LV (red circle), but the bolus was actually delivered in the extra-cardial space. US imaging detected this error in real time.

FIG. 2.26 illustrates the integration of MSCs with LV Wall. FIG. 2.26A illustrates a subset of mouse electrocardiogram after injection of a SiNP-labeled MSC bolus (500,000 MSCs). This section was chosen because it was in between respiratory cycles. FIG. 2.26B illustrates the signal inside an AOI was measured as a function of time (frame rate number). Clear correlation between phase of electrocardiogram and location of the MSC bolus is seen.

FIG. 2.27 illustrates Table S1 Cytokine analysis of MSC secretome. 31 different proteins were measured in the cell culture media of MSCs (column “Normal”) and SiNP-MSCs (column “SiNP”). Culture medium with no MSCs was also analyzed (column “Media”). The limit of detection (LOD) of the assay is presented as well as the coefficient of variation (CV) in the assay at the given concentration regime. Values of p were calculated to determine whether the increase was statistically significant using p=0.05 as the discriminating value.

FIG. 3.1 is a graph that illustrates an increase in ultrasound contrast over time after intravenous injection of particles. This figure shows signal increase in a U87MG xenograft tumor after injection of particles. A 12.5% signal increase is realized.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of imaging, chemistry, material science, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

The term “detectable” and “detection” refer to the ability to observe a specific signal over the background signal.

The term “ultrasound” or ‘US’” describes the delivery of sound waves to a sample. Ultrasound may involve the collection of sound waves for the purpose of creating and image. The term “B-mode” describes an ultrasound imaging technique that uses the reflection or backscatter of introduced ultrasound waves. The term “contrast mode” describes a method of ultrasound detection that utilizes resonance of a contrast agent to generate an image.

The term “photoacoustic imaging” describes signal generation caused by a light pulse, absorption, and expansion of a contrast agent, followed by acoustic detection, where the contrasting agent absorbs the light energy and converts it to thermal energy that generates the photoacoustic signal. The term “acoustic detectable signal” is a signal derived echo resulting from a particle (e.g., silica nanoparticle) that interacts with the matter (e.g., tissue) around the particle. The acoustic detectable signal is detectable and distinguishable from other background acoustic signals that are generated from the subject or sample. In other words, there is a measurable and statistically significant difference (e.g., a statistically significant difference is enough of a difference to distinguish among the acoustic detectable signal and the background, such as about 0.1%, 1%, 3%, 5%, 10%, 15%, 20%, 25%, 30%, or 40% or more difference between the acoustic detectable signal and the background) between acoustic detectable signal and the background. Standards and/or calibration curves can be used to determine the relative intensity of the acoustic detectable signal and/or the background. The term “echogenicity” is the amount of acoustic detectable signal that a sample possesses. The term “hyperechoic” describes areas of increased B-mode ultrasound.

The term “acoustic signal” refers to a sound wave produced by one of several processes, methods, interactions, or the like, that provides a signal that can then be detected and quantitated with regards to its frequency and/or amplitude. The acoustic signal can be generated from or modulated by one or more particles of the present disclosure. In an embodiment, the acoustic signal may be the sum of each of the individual ultrasound or photoacoustic signals. In an embodiment, the acoustic signal can be generated from a summation, an integration, or other mathematical process, formula, or algorithm, where the acoustic signal is from one or more probes. In an embodiment, the summation, the integration, or other mathematical process, formula, or algorithm can be used to generate the acoustic signal so that the acoustic signal can be distinguished from background noise and the like. It should be noted that signals other than the acoustic signal can be processed or obtained is a similar manner as that of the acoustic signal.

The acoustic signal can be detected and quantified in real time using an appropriate detection system. For example, two instruments that can be used quantifying acoustic signal are the Vevo 700 and Vevo 2100 from Visualsonics Corp. Others can be used and these can be purchased from manufacturers such as Philips, Siemens and General Electric. The units of acoustic signal can vary and include echogenicity units (EU) or mean grey scale. Input units include dB and frequency (MHz.) Maximum intensity persistence imaging can also be used and is described in Investigative Radiology: March 2011-Volume 46-Issue 3-pp 187-195. Other detection strategies including capacitive micromachined ultrasonic transducers (CMUT) arrays can also be used to detect the acoustic signal. Instruments suitable for detection photoacoustic imaging include instruments from Endra (Nexus 128) and Visual Sonics (LAZR).

The term “photoacoustic detectable signal” is a signal derived the contrasting agent absorbing light energy and converting it to thermal energy that generates the photoacoustic signal. The photoacoustic detectable signal is detectable and distinguishable from other background photoacoustic signals that are generated from the subject or sample. In other words, there is a measurable and statistically significant difference (e.g., a statistically significant difference is enough of a difference to distinguish among the photoacoustic detectable signal and the background, such as about 0.1%, 1%, 3%, 5%, 10%, 15%, 20%, 25%, 30%, or 40% or more difference between the photoacoustic detectable signal and the background) between photoacoustic detectable signal and the background. Standards and/or calibration curves can be used to determine the relative intensity of the photoacoustic detectable signal and/or the background.

The term “magnetic resonance detectable signal” is detectable signal is detectable and distinguishable from other background magnetic resonance signals that are generated from the subject or sample. In other words, there is a measurable and statistically significant difference (e.g., a statistically significant difference is enough of a difference to distinguish among the magnetic resonance detectable signal and the background, such as about 0.1%, 1%, 3%, 5%, 10%, 15%, 20%, 25%, 30%, or 40% or more difference between the magnetic resonance detectable signal and the background) between magnetic resonance detectable signal and the background. Standards and/or calibration curves can be used to determine the relative intensity of the magnetic resonance detectable signal and/or the background.

The term “optical detectable signal” (e.g., a fluorescent signal) is a signal derived from a particle that absorbs light and converts absorbed energy into optical energy of a different wavelength. The optical detectable signal is detectable and distinguishable from other background optical signals that are generated from the subject or sample. In other words, there is a measurable and statistically significant difference (e.g., a statistically significant difference is enough of a difference to distinguish among the optical detectable signal and the background, such as about 0.1%, 1%, 3%, 5%, 10%, 15%, 20%, 25%, 30%, or 40% or more difference between the ultrasound detectable signal and the background) between ultrasound detectable signal and the background. Standards and/or calibration curves can be used to determine the relative intensity of the ultrasound detectable signal and/or the background.

The term “dispose” describes the permanent or temporary attachment of matter to a supporting material.

The term “illuminating” as used herein refers to the application of a light source, including near-infrared (NIR), visible light, including laser light capable of exciting dyes and nanoparticle cores of the embodiments of the probes herein disclosed.

The term “in vivo imaging” as used herein refers to methods or processes in which the structural, functional, or physiological state of a living being is examinable without the need for a life ending sacrifice.

The term “aggregate” as used herein refers to a distance decrease between particles. In an embodiment, interaction between particles may occur. Aggregates of particles may have two or more individual particles combined into one grouping. Aggregation may occur spontaneously in a sample or subject or it may be controlled by a chemical or biological process. Aggregation may occur after injection of individual particles or delivery of individual particles to sample or particles may be aggregated prior to delivery.

The term “particle” or “nanoparticle” as used herein refers to a contrast agent with dimensions of about 1 and 5000 nm.

The term “silica” as used here refers to a structure containing at least the following the elements: silicon and oxygen. Silica may have the fundamental formula of SiO₂ or it may have another structure including SiₓO_{y} (where x and y can each independently be about 1 to 10). Additional elements including, but not limited to, carbon, nitrogen, sulfur, phosphorus, or ruthenium may also be used. Silica may be a solid particle or it may have pores. The silica may also be doped with material to increase its photoacoustic signal such as gold, silver, platinum, carbon nanotubes, or other solid materials. Small molecule dye, quenchers, or fluorophores may also be incorporated to increase photoacoustic signal.

The term “phantom” as used herein refers to a specimen created for the purposes of measuring acoustic detectable signal or other signal. These phantoms may be made of agar and other material.

The term “non-invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The term “sample” can refer to a tissue sample, cell sample, a fluid sample, and the like. The sample may be taken from a subject. The tissue sample can include brain, hair (including roots), buccal swabs, blood, saliva, semen, muscle, or from any internal organs, or cancer, precancerous, or tumor cells associated with any one of these. The fluid may be, but is not limited to, urine, blood, ascites, pleural fluid, spinal fluid, and the like. The body tissue can include, but is not limited to, brain, skin, muscle, endometrial, uterine, and cervical tissue or cancer, precancerous, or tumor cells associated with any one of these. In an embodiment, the body tissue is brain tissue or a brain tumor or cancer. In another embodiment, the body tissue is the heart or cardiac tissue or muscle tissue.

The term “administration” refers to introducing a probe of the present disclosure into a subject (e.g., a living subject). One preferred route of administration of the compound is oral administration. Another preferred route is intravenous administration. However, any route of administration, such as topical, subcutaneous, peritoneal, intraarterial, inhalation, vaginal, rectal, nasal, introduction into the cerebrospinal fluid, or instillation into body compartments can be used. Administration may also take place via any type of catheter. This delivery may be through a catheter placed in the coronary arteries. Intra-cardiac delivery is another route of administration and involves placing a delivery tool such as a catheter into the cardiac tissue.

The term “unit dosage form,” as used herein, refers to physically discrete units suitable as unitary dosages for human and/or animal subjects, each unit containing a predetermined quantity of a compound calculated in an amount sufficient (e.g., weight of host, disease, severity of the disease, etc.) to produce the desired effect in association with a pharmaceutically acceptable diluent, carrier or vehicle. The specifications for unit dosage forms depend on the particular compound employed, the route and frequency of administration, and the effect to be achieved, and the pharmacodynamics associated with each compound in the host. A unit dosage form can also be considered to be a discreet number of therapeutic cells or stem cells.

The term “therapeutically effective amount” as used herein refers to that amount of an embodiment of the agent being administered that will relieve to some extent one or more of the symptoms of the disease or condition being treated, and/or that amount that will prevent, to some extent, one or more of the symptoms of the disease or condition that the subject being treated has or is at risk of developing.

As used herein, “treat”, “treatment”, “treating”, and the like refer to acting upon a disease, condition, or disorder with an agent to affect the disease, condition, or disorder by improving or altering it. The improvement or alteration may include an improvement in symptoms or an alteration in the physiologic pathways associated with the disease, condition, or disorder. “Treatment,” as used herein, covers one or more treatments of a disease, condition, or disorder in a subject (e.g., a mammal, typically a human or non-human animal of veterinary interest), and includes: (a) reducing the risk of occurrence of the disease in a subject determined to be predisposed to the disease, condition, or disorder but not yet diagnosed (b) impeding the development of the disease, condition, or disorder, and/or (c) relieving the disease, condition, or disorder, e.g., causing regression of the disease, condition, or disorder and/or relieving one or more disease, condition, or disorder symptoms.

As used herein, the terms “prophylactically treat” or “prophylactically treating” refers completely or partially preventing (e.g., about 50% or more, about 60% or more, about 70% or more, about 80% or more, about 90% or more, about 95% or more, or about 99% or more) a disease, condition, or disorder thereof and/or may be therapeutic in terms of a partial or complete cure for a disease, condition, or disorder and/or adverse effect attributable to the disease, condition, or disorder.

As used herein, the term “host,” “subject,” or “patient,” includes humans and mammals (e.g., mice, rats, pigs, cats, dogs, and horses). Typical subject to which particles of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above that is alive. The term “living host” refers to the entire host or organism and not just a part excised (e.g., a liver or other organ) from the living host.

### General Discussion

Embodiments of the present disclosure provide for nanoparticles for acoustic imaging, methods of using the nanoparticles, methods of imaging a condition, and the like. Embodiments of the present disclosure include nanoparticles (e.g., silica nanoparticles) that can be used to image, detect, study, monitor, evaluate, and/or screen a sample or subject (e.g., whole-body or a portion thereof).

Regenerative medicine has yet to be completely realized in part because of insufficient imaging modalities to track the location(s), number, and differentiation of implanted stem cells. Ultrasound (US) is an especially promising technology due to its broad access, high resolution, low cost, and depth penetration. Unlike PET and MRI, US facilitates the real-time guidance of stem cell implantation in cardiac and abdominal applications. Unfortunately, US is challenged by a lack of effective probes.

The gold standard in ultrasound contrast agents is the microbubble. While the microbubble offers intense signal, it is very large (1-5 micrometers) and is only stable for hours. Although microbubbles have been used for vascular applications, their large size prevents routine cell labeling, which is limited to the cell exterior.

Nanoparticles, such as silica nanoparticles, of the present disclosure are stable for many days and are an order of magnitude smaller (e.g., 200 nm). Because of their small size, they can be loaded inside the cell, rather than only on the exterior (like microbubbles). The microbubbles may not be truly labeled into the cell, especially after the many manipulations involved in stem cell implantation. Thus, with silica nanoparticles, for example, the clinician can be certain the increased signal is from the cells, not free contrast agent. Further, their long term stability can allow clinicians to monitor the cells over days or weeks.

In an embodiment, silica nanoparticles can be used as a US contrast agent, in particular, they can be used to image cells such as mesenchymal stem cells (MSCs) in living subjects. The silica nanoparticles offer intense, stable, and optionally multimodal signal, in the B-mode and/or contrast mode with potentially biodegradable byproducts. In an embodiment, the silica nanoparticles can be about 200 nm (e.g., about 25 to 5000 nm) in diameter (or longest dimension if not a sphere) nanoparticles and are compatible with 16-40 MHz ultrasound. Lower ultrasound frequencies (e.g., 5-12 MHz, or other frequencies) more compatible with human imaging may also be used. In addition, the silica nanoparticles can have an embedded fluorescent tag to label the cell (e.g., mesenchymal stem cells). In an embodiment, the silica nanoparticles can be imaged on the cell interior with light and electron microscopy. The tagged cells are easily discriminated from untagged cells by 16 MHz B-mode ultrasound. Use of embodiments of the present disclosure appears to be the first demonstration of in vivo cell tracking with microbubble-free US.

For applications beyond cell implantation, this small size may facilitate extravasation from the vascular space, which is impossible with the micrometer-sized microbubbles. These silica nanoparticles are useful alternatives to microbubbles for ultrasound contrast because of their size (nm) and stability (months) and could have additional applications beyond cell tracking.

In an embodiment, the silica particles can have a complementary signaling mode. In another embodiment, the silica nanoparticles can have other imaging modes such as photoacoustic, PET, and/or MRI by including photoacoustic absorbers, PET, or MRI modalities in, part of, or attached to the silica nanoparticles. For example, an iron oxide core/silica shell can be used for tandem ultrasound/MRI imaging. In another example, a gold core/silica shell can be used for tandem ultrasound/photoacoustic imaging. In another embodiment, silica particles with a fluorescent tag embedded inside can be used for both flow cytometry sorting before implantation and ultrasound imaging during/after implantation or for general imaging (i.e., administered separate from the cell). In another embodiment, silica particles can also be radiolabeled for nuclear imaging.

Besides stem cell tracking, silica particles could be a replacement for a variety of other applications currently reserved for microbubbles including imaging tumor via molecular targets. One fundamental advantage of the present disclosure will be that these particles could potentially leave the blood vessels and lodge in tumor. This is impossible with microbubbles because of their size.

An embodiment of the silica nanoparticles can be advantageous in that the silica nanoparticles can be imaged in real-time. Current approaches to imaging implanted cells involve MRI or PET imaging, which unfortunately cannot be done while the cells are being implanted (real time imaging). Imaging during implantation is critical to ensure that the cells are in the proper location. Embodiments of the present disclosure offer ultrasound contrast, which is a real time imaging modality. In addition, the silica nanoparticles are advantageous in that they do not use air to generate ultrasound contrast. Air in the vascular space can be problematic. The silica nanoparticles are sub-micron (e.g., less than 500 nm) and offer safety, cost, and intensity advantages over microbubbles. Furthermore, existing ultrasound contrast agents are limited to vascular targets because of their size. Silica nanoparticles may leave the circulation to image targets outside the vascular space, i.e., they may label cell surface markers on cells in the parenchyma or bulk of a tumor or organ, not only the vascular space.

In an embodiment, silica nanoparticles can be made of entirely silica or the silica can be a layer or coating disposed on a nanoparticle (e.g., a metal nanoparticle such as a sphere, rod, and the like). In an embodiment, the silica nanoparticle can have one or more agents (e.g., targeting agent, fluorescent agent, and the like) disposed on the silica nanoparticle.

In an embodiment, the silica nanoparticles can be made using a synthesis (e.g., a modified Stober approach, for example see Langmuir 2005, 21, 4277-4280 and Langmuir 2005, 21, 7524-7527) involving water, ethanol, strong base, and a tetraethylorthosilicate precursor. The diameter or the effective diameter (e.g., if the nanoparticle is rotated about a center of rotation (e.g., to form an imaginary sphere) and the outer most distance from the center of rotation reached (the edges of the imaginary sphere) defines the effective diameter) can be about 20 nm to 5000 nm or a radius of about 10 to 2500 nm. The dimensions of the silica nanoparticle are measured using transmission electron microscopy. For example, about 1 microliter of particle sample with optical density of about 0.1 is placed on a formvar grid from Ted Pella, dried, and imaged under standard conditions with a Tecnai microscope. Diameter is also measured by dynamic light scattering via a Zetasizer instrument from Malvern. Here, 1.5 mL of the same sample with the optical density mentioned above is analyzed using software from Malvern. When the particles are imaged in a planar fashion, the circularity of the particles may have values from 0.5 to unity. Here, circularity=4 pi(area/perimeter̂2) and is measured with ImageJ software from the National Institutes of Health. In addition, the diameter or the effective diameter can be tuned based on concentrations of starting materials.

In an embodiment, the silica particles may be porous to modulate ultrasound contrast. This can be done by adding surfactants or other polymers such as cetyltrimethylammonium bromide, hexadecyltrimethylammonium bromide, or mesitylene to the reaction mixture. The porosity may be measured by the size of the pores in the particles (1-20 nm) by electron microscopy or atomic force microscopy or by the surface area of the particles (10-1200 m²/g). Surface area may be measured by a surface area analyzer using BET theory (see Adsorption of Gases on Heterogeneous Surfaces by Rudzinski and Everett and J. Am. Chem. Soc., 1938, 60, 309).

In an embodiment, the silica nanoparticles can either be spherical, rod-like, or irregularly shaped. The aspect ratio (defined as width/height) may be about unity for spherical particles to 10. In an embodiment, aggregation of silica nanoparticles (e.g., 2 to 2000) can be used to modulate the signal intensity.

As noted above, the silica nanoparticles can include a core and a silica layer or coating (e.g., about 1 to 100 nm, about 1 to 50 nm, or about 1 to 20 nm, thick). In an embodiment, the surface can be coated (e.g., a thickness of one monolayer to hundreds of layers and coverage of about 1 to 100%). This coating may involve a trimethoxysilane (TMS) group to bind to the silica surface with any number of pendant groups attached to the TMS. This organosilane may have, but is not limited to, a mercapto, amino, carboxyl, cyano, azido, alkyne group, or a combination thereof. It may also have a polyethylene glycol or polyethylimine constituent to modulate cellular uptake. These groups may be further modified to attach a targeting ligand through standard bioconjugation chemistry. Additional details are provided in the Examples.

In an embodiment, silica nanoparticles can be used to track cells (e.g., stem cells, induced pluripotent stem cells, embryonic stem cells, donor marrow cells, other donor cells, cancer cells, cancer stem cells, donor tissue, or regenerated tissue) or other agents (e.g., medical devices, catheters, needles, or stents) implanted into a living subject. In an embodiment, where cells are destined for implantation, the cells can be incubated with the silica nanoparticles and incorporate the silica nanoparticles into the cytoplasm, for example. Subsequently, the cells are harvested and introduced to the injured site. In another embodiment, the cells loaded with nanoparticles can be injected intravenously. The cells might then traffic to a site of interest such as an inflamed region, an infected region, or a region of chornic disregulation such as a tumor. The nanoparticle inside the cell will then report signal. As mentioned above, the silica nanoparticles can enhance the B-mode and/or photoacoustic contrast mode or MRI contrast of imaging of the implanted cells and allow the surgeon to guide the cells to the correct location. Embodiments of the present disclosure can be used in applications such as cancer, precancerous tissue, tumors, cardiology, echocardiology, cardiovascular medicine, regenerative medicine, cell therapy, imaging, orthopedics, internal medicine, radiology, interventional radiology, obstetrics/gynecology, urology, and oncology.

In another embodiment, the silica nanoparticles can be used to image organs or tumors by accumulation after intra-venous injection. The silica nanoparticles can be coated with a targeting agent having an affinity for a target. In particular, the targeting agent can bind to integrins. In an embodiment, the silica nanoparticles can be injected intravenously and then accumulate specifically at the diseased area for ultrasound imaging. In addition, the silica nanoparticles can include a therapeutic agent that can be used to treat a condition or disease. The release of this material may be controlled by tuning the size and charge of the nanoparticle and/or the nanoparticle pores. Therapeutic agents may treat the living subject to which they are administered. The therapeutic agent may also treat the cell or stem cell that contains the nanoparticle. In addition, ultrasound ablation can increase the release of therapeutic agents embedded in the silica nanoparticles.

As mentioned above, embodiments of the method include disposing one or more cells in a subject. The cells include one or more silica nanoparticles within the cell. In an embodiment, the silica nanoparticles can include one or more other imaging modalities and/or a therapeutic agent. While and after the cells are disposed in the subject, the subject and the cells can be imaged to determine the placement of the cells within the subject. A signal (e.g., acoustic signal) can be detected during the placement of the cells and/or after placement of the cells. The detected signal can be used to correlate the position of the cells within the subject. In an embodiment, the imaging is conducted using an ultrasound device, endoscopic ultrasound device, intravascular ultrasound device, or ultrasound device utilizing a capacitive micromachined ultrasonic transducer (e.g., Vevo 700, Vevo 2100, or General Electric Vivid 7 or any other clinical/preclinical systems or endoscopic designs). In an embodiment, a 2-dimensional, pulse-wave Doppler, continuous-wave Doppler, or color Doppler imaging can be used. Use of an ultrasonic device is advantageous in that the signal can be detected in real-time. If additional modalities are used, other devices can be used to image (e.g., photoacoustic device, MRI device, fluorescent detection system, nuclear detection system, and the like). In each of these, the signal (e.g., MRI signal, fluorescent signal, nuclear imaging signal, etc.) can be used to correlate the position of the cells within the subject.

In another embodiment, the present disclosure provides for a method of imaging. In an embodiment, silica nanoparticles of the present disclosure can be introduced (e.g., administered) to a subject where the silica nanoparticle may include a targeting agent having an affinity for a target. Passive targeting may also be used. After an appropriate amount of time, the subject is exposed to one or more imaging devices so that a signal(s) (e.g., ultrasonic signal) can be detected. The location of the target can be correlated with the location of the detected signal(s).

In another embodiment, the present disclosure provides for a method of administering to a subject in need of treatment a therapeutically effective amount of an agent. The therapeutic agent is attached to a silica nanoparticle. In addition, the silica nanoparticle includes a targeting agent having an affinity for a target (e.g., a disease, condition, or a compound (e.g., protein, cancer, etc.) associated with the disease or condition). Silica nanoparticles of the present disclosure can be introduced (e.g., administered) to a subject. After an appropriate amount of time, the subject is exposed to one or more imaging devices so that a signal(s) (e.g., ultrasonic signal) can be detected. The location of the target can be correlated with the location of the detected signal(s). Once the location of treatment is known (e.g., the location of the silica nanoparticles since the targeting agent has an affinity for the area (e.g., protein, cancer, etc.) to be treated), an ultrasonic ablation can be performed to release the therapeutic agent at the desired location.

As mentioned above, the silica nanoparticle can include one or more agents (e.g., a chemical or biological agent). In an embodiment, the agent can include a targeting agent, a drug, a therapeutic agent, a radiological agent, a chemological agent, a small molecule drug, a biological agent (e.g., peptides, proteins, oligomers, antibodies, antigens, and the like), and combinations thereof. In an embodiment, each agent can be disposed indirectly or directly on the silica nanoparticle.

In an embodiment, the targeting agent has an affinity for a target in a subject or a tissue or fluid sample from a subject. In particular, the agent enables the nanoparticle to be used to image, detect, study, monitor, evaluate, and/or screen a disease, condition, or related biological event corresponding to the target.

In an embodiment, the targeting agent can function to cause the silica nanoparticle to interact with a molecule(s) or protein(s) or other target. In an embodiment, the targeting agent can have an affinity for a cell, a tissue, a protein, DNA, RNA, an antibody, an antigen, and the like, that may be associated with a condition, disease, or related biological event, of interest. In particular, the targeting agent can function to target specific DNA, RNA, and/or proteins of interest. The targeting agent can include, but is not limited to, polypeptides (e.g., proteins such as, but not limited to, antibodies (monoclonal or polyclonal)), antigens, nucleic acids (both monomeric and oligomeric), polysaccharides, sugars, fatty acids, steroids, purines, pyrimidines, ligands, aptamers, small molecules, ligands, or combinations thereof, that have an affinity for a condition, disease, or related biological event or other chemical, biochemical, and/or biological events of the condition, disease, or biological event. In an embodiment, the targeting agent can include: sequence-specific DNA oligonucleotides, locked nucleic acids (LNA), and peptide nucleic acids (PNA), antibodies, and small molecule protein receptors.

Embodiments of the present disclosure can be used to image, detect, study, monitor, and/or evaluate, a condition or disease such pre-cancerous tissue, cancer, or a tumor. In an embodiment, the method includes imaging pre-cancerous tissue, cancer, or a tumor. In particular, the silica nanoparticles of the present disclosure can be used to image a tumor since the silica nanoparticles can enter the tumor since they are relatively small. A method can include exposing a subject to an imaging device (e.g., ultrasound detection system and/or ultrasound source system). The subject is given the silica nanoparticles prior to exposure and/or during exposure to the imaging device and after a period of time the particle enters the tumor. Subsequently, the silica nanoparticles are detected and the location of the silica nanoparticles can be determined. The location of the silica nanoparticles can be correlated with the location of the tumor and/or the presence of the tumor. Additional details are described in Examples.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Brief Introduction

Improved imaging modalities are critically needed for optimizing stem cell therapy. Techniques with real-time content to guide and quantitate cell implantation are especially important in applications such as musculoskeletal regenerative medicine. Here, we report the use of silica-coated gold nanorods as a contrast agent for photoacoustic imaging and quantitation of mesenchymal stem cells in rodent muscle tissue. The silica coating increased the uptake of gold into the cell more than 5-fold, yet no toxicity or proliferation changes were observed in cells loaded with this contrast agent. Pluripotency of the cells as retained, and secretome analysis indicated that only IL-6 was disregulated more than 2-fold from a pool of 26 cytokines. The low background of the technique allowed imaging of down to 100,000 cells in vivo. The spatial resolution is 340 μm, and the temporal resolution is 0.2 s, which is at least an order of magnitude below existing cell imaging approaches. This approach has significant advantages over traditional cell imaging techniques like positron emission tomography and magnetic resonance imaging including real time monitoring of stem cell therapy.

### Introduction

The promises of stem cell therapy (SCT) for musculoskeletal disease such as muscular dystrophy including regeneration of myofibers have been hampered by poor survival of implanted cells.¹⁻⁷ A variety of stem cell types have been examined including satellite cells⁸ and mesenchymal stem cells (MSCs).⁹ MSCs have had few adverse events with generation of muscle cells.⁹⁻¹³

Specific limitations to stem cell therapy (SCT) include cell death, contamination by undifferentiated cells, and cell delivery to untargeted areas.¹⁴ In one of the first human examples of SCT, cells were mis-injected in 50% of patients.^{15, 16} In that study, cell imaging during injection could not be performed and the poor injection rates were not identified until post-procedure magnetic resonance imaging (MRI) analysis. Although local delivery improves accuracy, there is no way to image and quantitate the number of cells accumulating at the target site in real time. Indeed, it is currently unclear whether the lack of response observed in some SCT is due to poor biology or poor graft delivery.

Imaging is a fundamental tool to improve SCT and can assist with proper delivery of cells and also monitors the short-term and long-term fate of delivered cells.¹⁷ Such imaging is critical to determine the location and quantity of cells during the transplant event, but also the quantity and redistribution during tissue repair. There are two main approaches to stem cell imaging: 1) labeling with a reporter gene or 2) labeling with an exogenous contrast agent. Reporter genes for positron emission tomography (PET) and optical imaging are quantitative and offer content on cell proliferation, but are difficult to envision clinically due to depth limitation (optical) and the need for alteration of the stem cell machinery. Alternatively, iron oxide nanoparticles are used for cell imaging with MRI. MRI has excellent resolution, soft tissue contrast, and detection limits (10-20 cells/voxel).^{15, 17-20} MRI cell tracking was reported nearly a decade ago and is currently capable of single cell imaging^{21, 22}. Unfortunately, both MRI and PET have temporal resolution of minutes, which precludes them from use during the cellular implantation event.

One alternative approach to these established techniques is photoacoustic (PA) imaging. In photoacoustic imaging (PAI), ultrasound waves are generated via a pressure difference induced by the rapid heating from a nanosecond light pulse incident on the sample.²³⁻³⁰ PAI may either use endogenous contrast such as oxy- and deoxy-hemoglobin³¹ or exogenous contrast agents such as small molecules,³² carbon nanotubes,^{28, 33} or gold nanorods (GNRs).^{34, 35} PAI is used tangentially with normal backscatter mode (B-mode) ultrasound. It is quantitative, non-invasive, and has short scan times. It is an ideal tool to use for stem cell implantation because B-mode ultrasound will already be used to localize the delivery catheter near the diseased site. PAI can quantitate the implanted cells in real time to confirm that an adequate number of cells reach the treatment site.

In this Example, we use silica-coated GNRs (SiGNRs) as a PA contrast agent to label MSCs and image them in the musculature of living mice. Cellular uptake of the contrast agent is facilitated by the silica coat, which also increases the PA signal of the GNRs.³⁵⁻³⁷ We measured the effect of the SiGNRs on MSC viability, proliferation, differentiation, and cytokine expression. We imaged and quantitated MSCs in agarose phantoms, and finally injected labeled MSCs into the muscle of living mice to estimate in vivo detection limits.

### Results

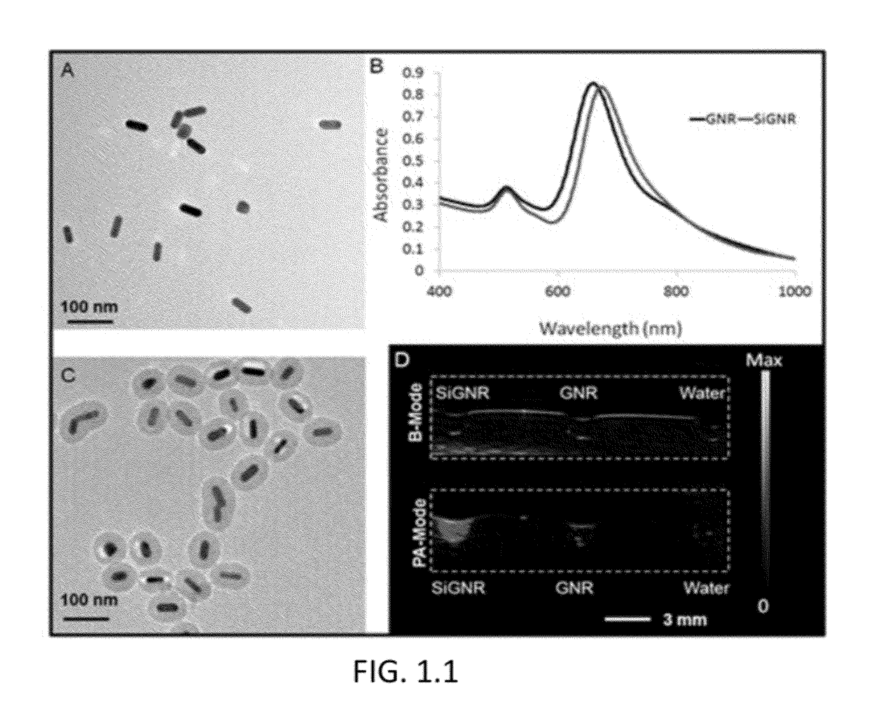

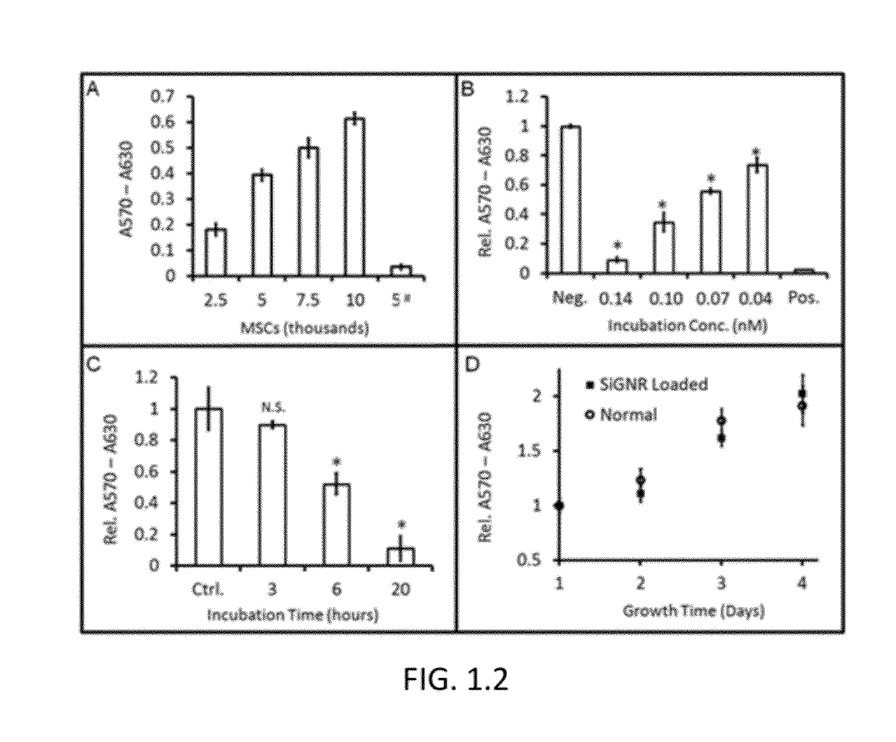

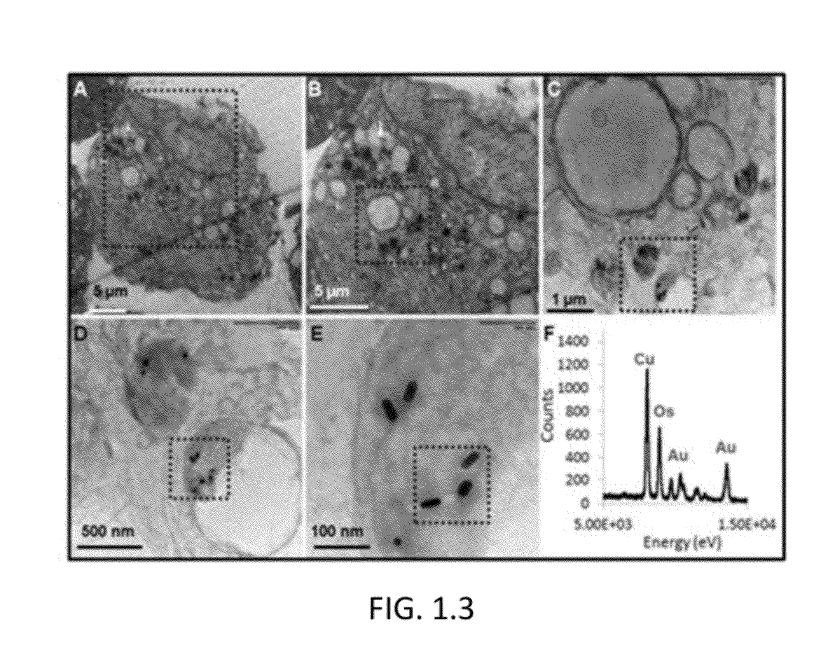

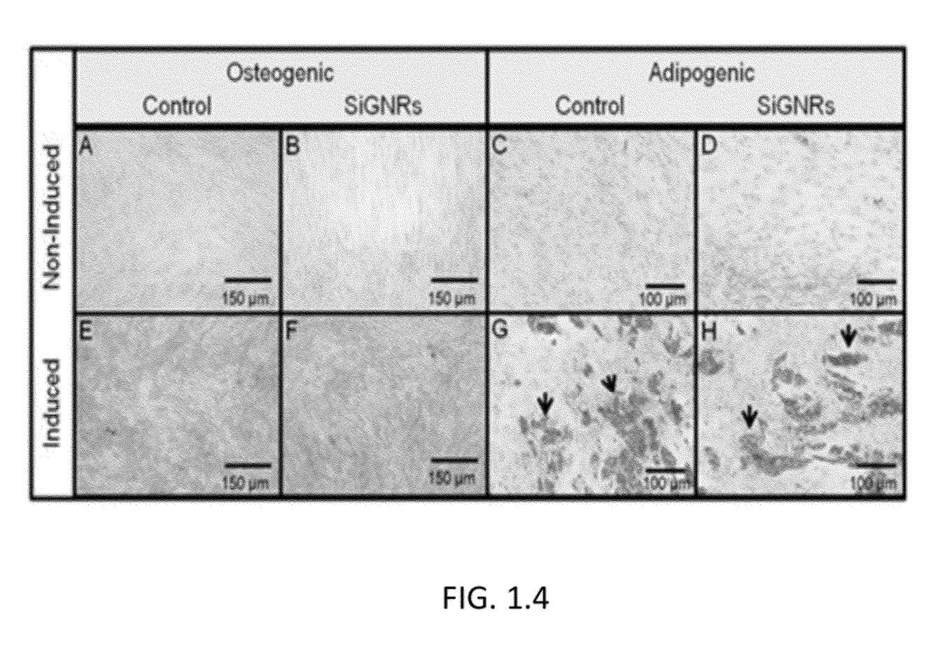

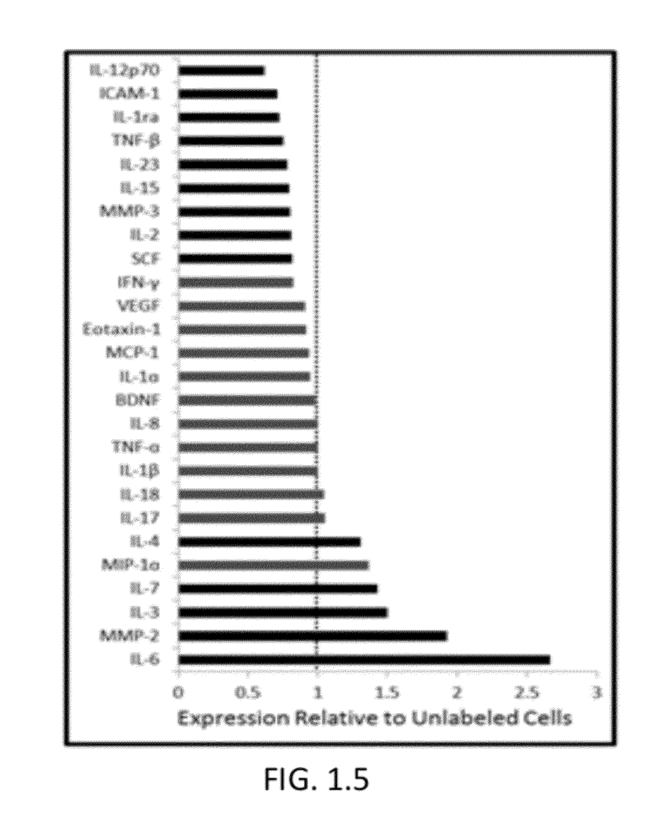

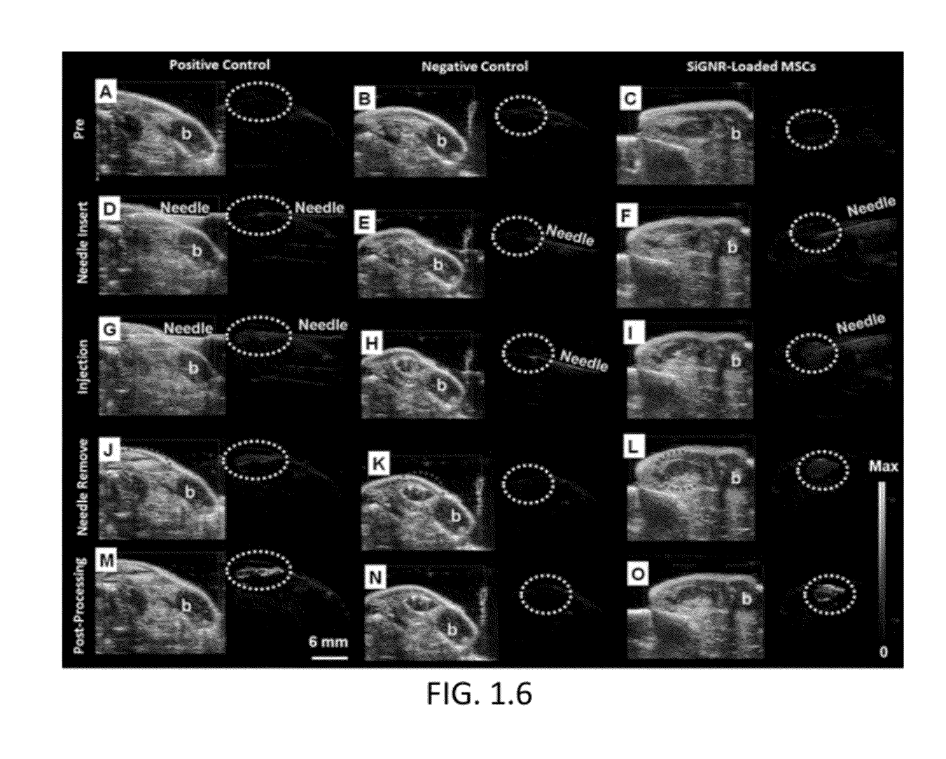

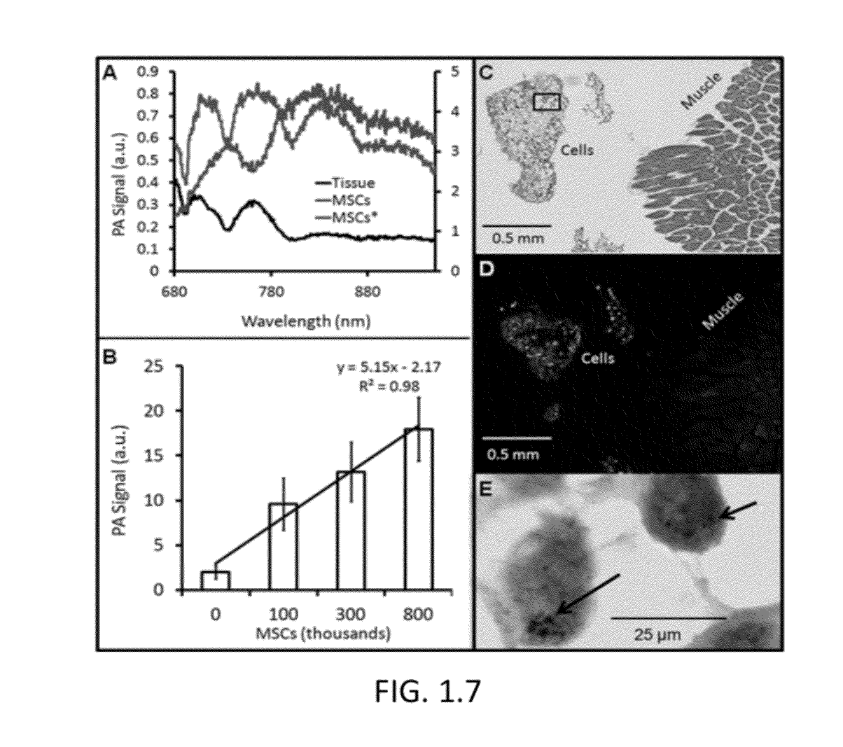

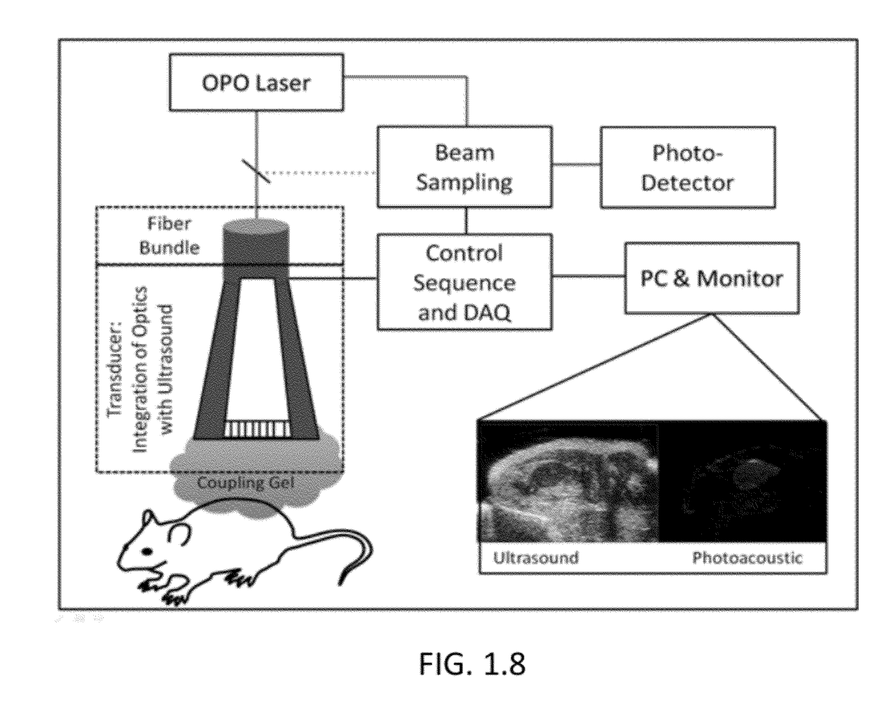

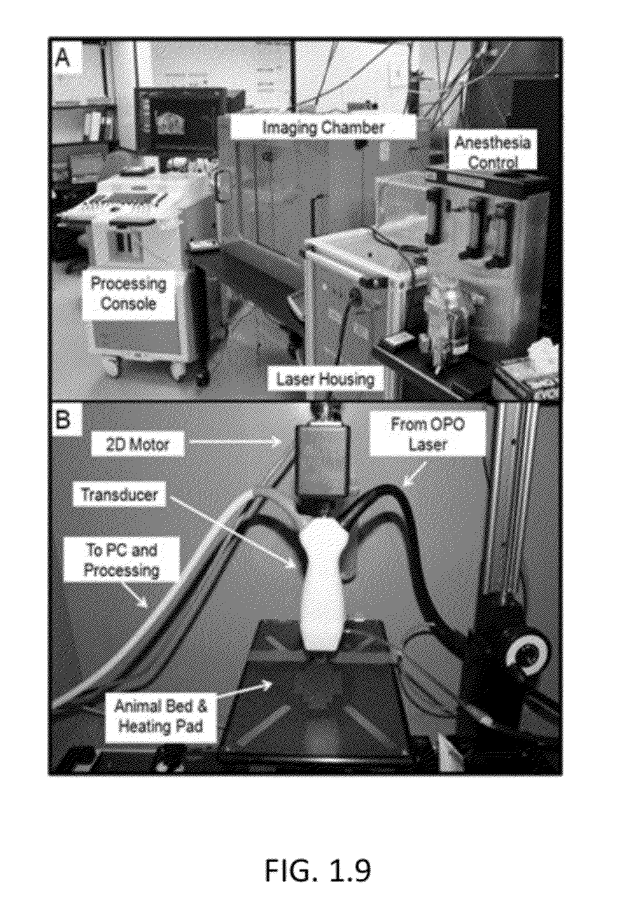

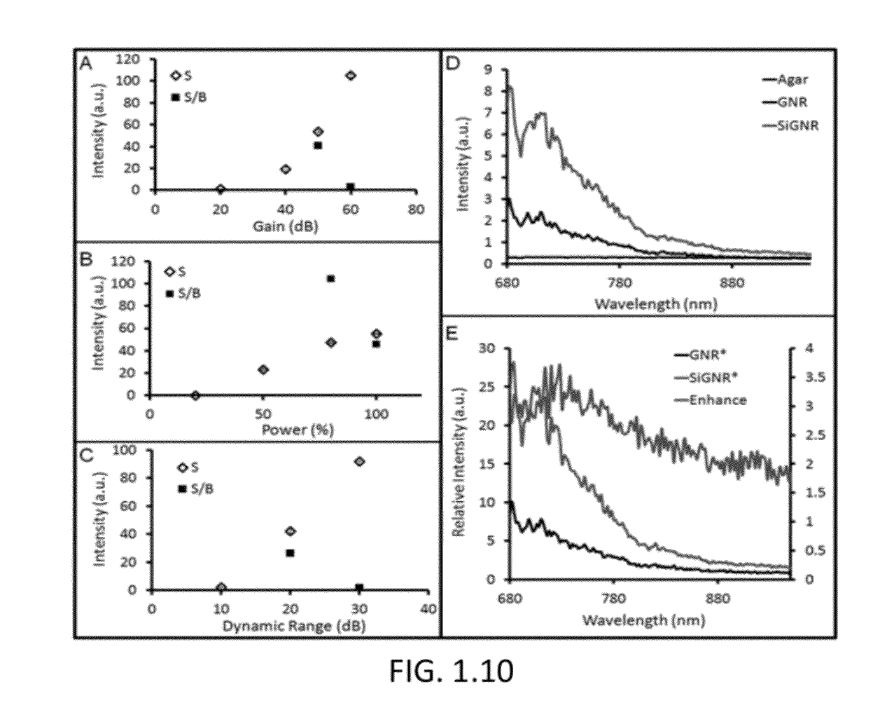

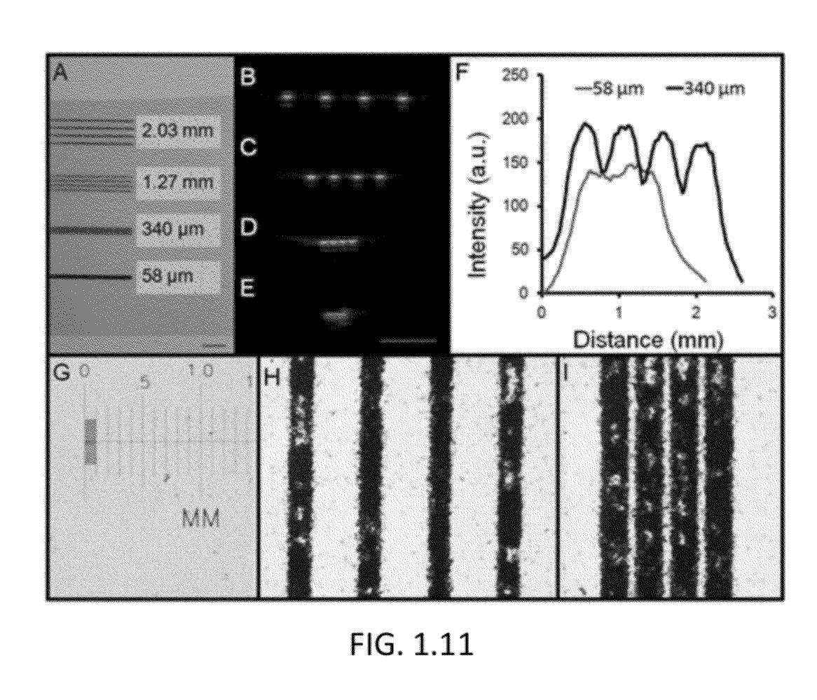

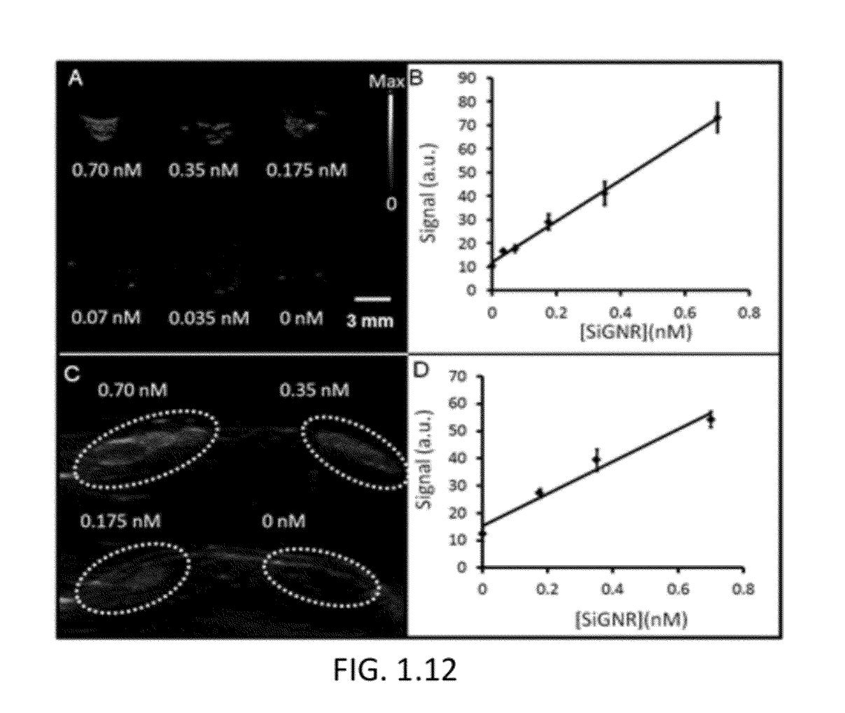

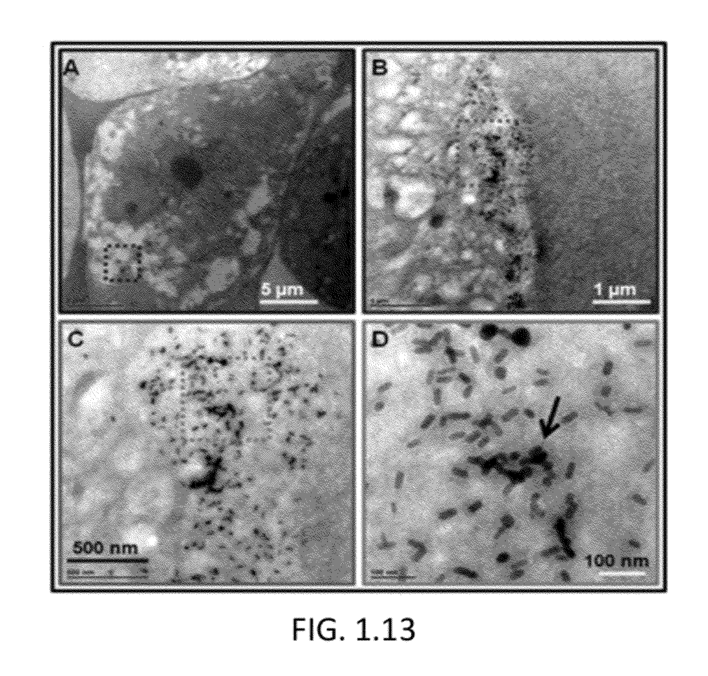

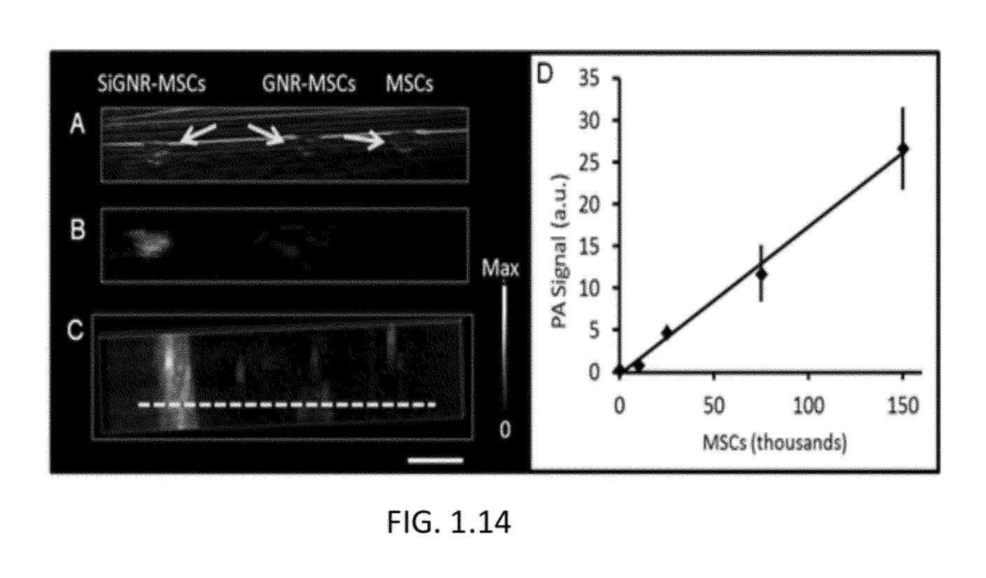

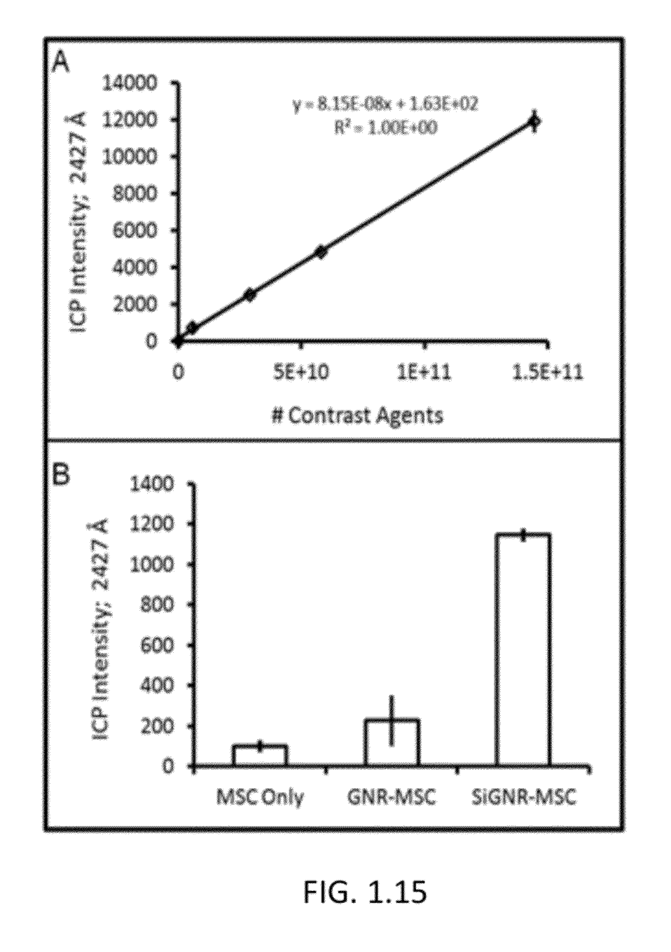

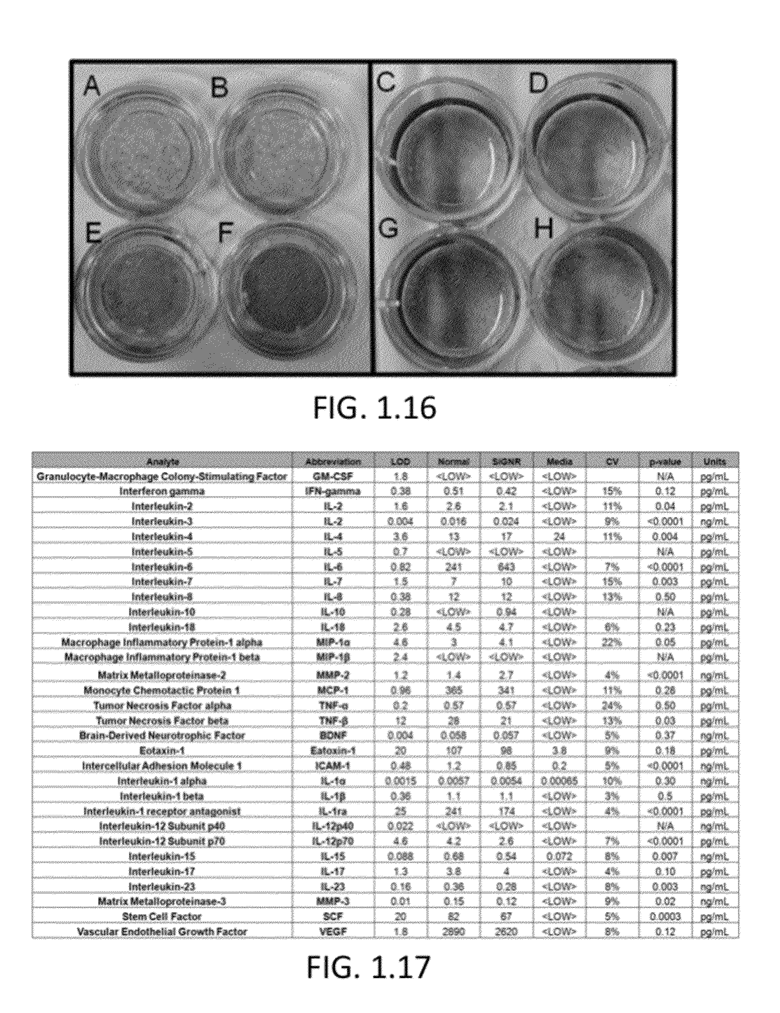

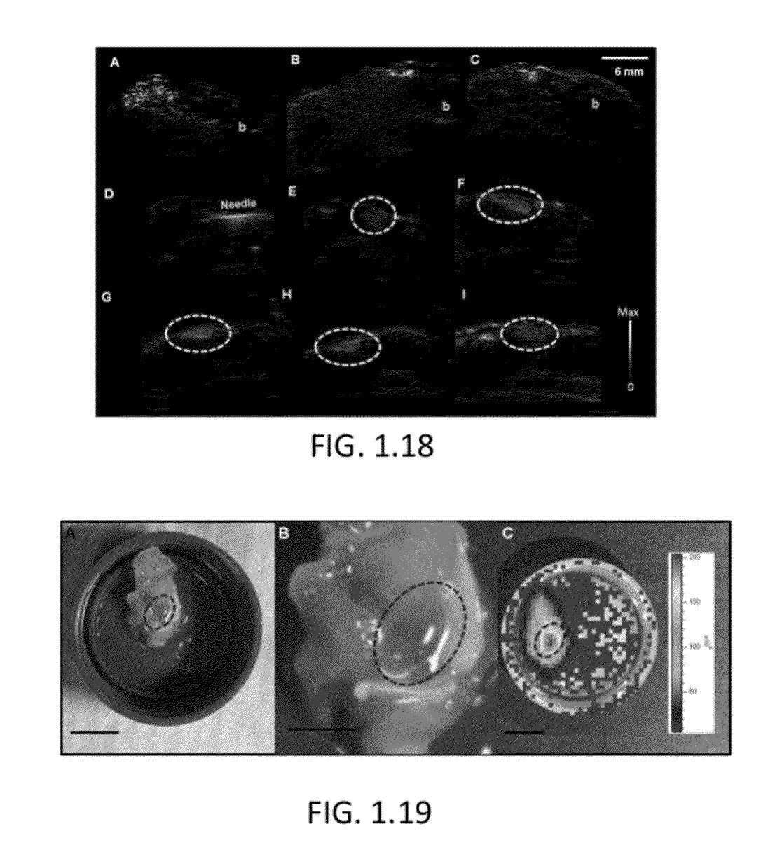

The GNRs and SiGNRs were characterized via TEM and absorbance spectroscopy (FIG. 1.1). The GNRs had a peak resonance at 665 nm with average dimensions of 42.17±5.11 nm by 14.90±0.58 nm as measured by TEM and ImageJ analysis (FIG. 1.1A). After silica coating (FIG. 1.1B), the dimensions increased to 82.99±3.86 by 64.20±3.48 nm width with an additional 11 nm in red-shift of the plasmon resonance to 676 nm (FIG. 1.1C). This 20 nm shell thickness was previously reported to be optimal for PA imaging.³⁵ DLS indicated that the GNRs had a charge of 14.7 mV and the SiGNRs were 7.8 mV in 1:1 PBS/water.³⁸ The PA signal of GNRs and SiGNRs at 1.4 nM was also calculated and the silica coating produced a 4-fold increase in PA signal (FIG. 1.1D). Previous reports suggest that silica coating provides a three-fold increase in PA signal.³⁵ The four-fold increase seen here is likely due to a closer matching of the SiGNR peak (676 nm) with the excitation pulse (680 nm) relative to the uncoated GNRs (665 nm).

The PA scanner consisted of three separate components including a light-tight imaging chamber, an excitation source, and a PC-based processing console (FIGS. 1.9 and 1.10) The imaging conditions (gain, power, and dynamic range) of the PA instrument for this contrast agent were empirically optimized. For additional details of these descriptors, please see caption of FIG. 1.11. The laser power was monitored with an external power meter as well as internal power sampling. At 680 nm, the average power detected 1 cm away from the transducer was 9.5 mJ (6.9-12.9 mJ) with root mean square variation of 10.1% for 500 pulses. FIG. 1.11 presents an experiment in which other parameters were sequentially modulated and the resulting signal from the contrast agent was plotted along with the signal-to-background ratio. Optimal conditions were achieved with a gain of 50 dB, 80% power, a persistence of four frames (no persistence was used for real time imaging), and 20 dB of dynamic range. These conditions were used for the remainder of the experiments. The spatial resolution was probed by imaging a test pattern printed on transparency film. Spacing of 340 μm was easily resolved while spacing of 58 μm could not be resolved (FIG. 1.12). There was a linear relationship (R²>0.99) between concentration (up to 0.7 nM) and PA signal of the SiGNRs in an agarose phantom with a LOD of 0.03 nM SiGNRs (FIG. 1.13A, 1.13B). No decrease in PA signal intensity was observed for the SiGNRs over 60 days.

The capacity of SiGNRs to label MSCs was studied next. Previously, silica has facilitated endocytosis into a variety of cell types, including MSCs.^{39, 40} To choose the appropriate starting concentration and incubation time of the SiGNRs, we used the MTT cell toxicity assays and centered the study near 0.05 nM SiGNRs, which has previously shown efficacy for cellular labeling with gold core/silica shell nanoparticles (FIG. 1.2).⁴¹ Both SiGNR concentration (FIG. 1.2B) and the incubation time (FIG. 1.2C) were studied. The results indicate that 0.07 nM of SiGNRs (1.5×10⁶ SiGNRs/MSC) at 3 hours of incubation time gave no statistically significant change in MSC metabolic activity relative to the negative control (p>0.05). To confirm SiGNR endocytosis, TEM images of fixed cells were acquired, and accumulation of SiGNRs inside MSC vesicles was noted (FIGS. 1.3 and 1.14). The silica coat was not entirely clear because the electron density of silica is approximately the same as the 400 nm section of resin.

The capacity of GNR- and SiGNR-labeled MSCs to generate PA signal was studied relative to nanoparticle free-MSCs, all at 50,000 cells (in 15 μL) in an agarose phantom (FIG. 1.15). Maximum intensity projections were created to analyze the data with ROI analysis. A 2.8-fold increase was measured for the GNR-labeled MSCs relative to unlabeled MSCs. The signal of GNR-MSCs was 7.6-fold lower than the same number of MSCs with SiGNRs (FIG. 1.15C). Theoretically, this increase should have been 20-fold (5 times more contrast with 4 times more signal). This lower observed signal is likely due to optical attenuation and scatter that occurs inside the MSC. The effect of incubation time on PA signal of MSCs was also measured for the 3, 6, and 20 hour time points at 0.07 nM. The 6 hour time point had the same (p=0.33) PA intensity as the 3 hour incubation sample, but the 20 hour sample was reduced. The PA signal of the 20 hour sample was 34% of the 3 hour sample. To determine the ex vivo LOD, we immobilized decreasing numbers of SiGNR-loaded MSCs into a phantom and collected PA images. The LOD above the water blank was 5,000 cells (FIG. 1.15B).

ICP analysis determined the amount of gold loaded into the cells. First, increasing number of GNRs and SiGNRs were analyzed for their gold content and that signal was plotted in FIG. 1.16A. This calibration plot was used with the gold content of dissolved MSCs to determine the number of SiGNRs present in the total sample as well as the quantity on a per-cell basis. We calculated 102,000±1,000 SiGNRs per MSC; the gold signal from SiGNR-loaded MSCs was 5-fold higher than that from GNR-loaded MSCs (FIG. 1.16C).

We performed additional studies to determine whether the SiGNRs altered the normal behavior of MSCs beyond gross toxicity assays like the MTT metabolic tests. First, to determine whether this loading changes the normal proliferation of MSCs, 3,000 MSCs with and without SiGNR loading were seeded in a 96 well plate and monitored sequentially with MTT. There was no significant (p=0.35) difference between the growth of the two cell populations (FIG. 1.2D). The doubling time for both populations was three days.

Next, we used differentiation reagents to determine whether the SiGNRs impacted the pluripotency of the MSCs.⁴² This work sought to answer two questions: 1) Can SiGNR-loaded MSCs still differentiate⁴³? and 2) Does the presence of SiGNRs induce any unintended differentiation? We were especially concerned that the SiGNRs might unintentionally transform MSCs into osteogenic cells as silicon-based structures have previously been show to induce such differentiation.⁴⁴ Fortunately, SiGNR-loaded cells were still easily transformed into osteogenic and adipogenic cell lines (FIG. 1.4). There was 5-fold more osteogenic signal (as determined by A402) in the induced (FIG. 1.4) cells than non-induced cells (FIG. 1.4B). Adipogenic induction produced many lipid-containing vacuoles in both the control (FIG. 1.4G) and SiGNR containing cells (FIG. 1.4H). See photographs of the culture plates in FIG. 1.17.

A final study analyzed the secretome of SiGNR-loaded and control MSCs⁴⁶. Of the 31 analyzed proteins, 26 had levels in cell culture media that were measureable by the bead-based Luminex assay; Table 1.17⁴⁶. We compared the levels in the labeled MSCs to unlabeled MSCs and found that only interleukin-6 (IL-6) had expression patterns increased or decreased more than 2-fold.

The utility of SiGNRs in living systems was first probed by implanting decreasing concentrations of SiGNRs (80 μL of 0.7, 0.35 and 0.175 nM in 50% matrigel) sub-cutaneously and performing PA imaging. The LOD for the SiGNR contrast agent in vivo (subcutaneous) was 0.05 nM and linear at R²=0.93 (FIG. 1.13B, 1.13D). The next step was to inject SiGNR-labeled MSCs. FIG. 1.6 presents representative sequences of intra-muscular cell implantation (FIG. 1.6, Right) including positive (FIG. 1.6, Left) and negative controls (FIG. 1.6, Middle). Images of hind limb muscle and images, during, and after injection are shown. For video of real-time injection of the SiGNR-labeled MSCs presented in the right of FIG. 1.6. The positive control is 3 nM SiGNRs only, the negative control is PBS, and the cell implantation is 800,000 cells. Importantly, the B-mode image shows the implant in all three examples (FIG. 1.6J, K, and L). The red dashed circle highlights the injection site. For FIG. 1.6K, there is clearly an i.m. bolus injection, but no PA signal. In contrast, FIG. 1.6L with SiGNR-MSCs shows a bolus and PA signal. Spectral analysis of the therapy site was performed before and after injection (FIG. 1.7A).

The difference pre and post injection at the injection site was 670% increase for positive control; no increase in PA signal was observed for the negative (vehicle) control. Decreasing numbers of SiGNR-labeled MSCs (8×10⁵-1×10⁵) were delivered into a mouse hind limb muscle in three replicate mice at each different cell number (FIG. 1.7B and FIG. 1.10) The lowest value imaged was 100,000 cells and the calculated in vivo LOD of MSCs in mouse hind limb muscle is 90,000 cells. One animal with 100,000 cells was monitored longitudinally and a PA image recorded daily. The implanted cell bolus could be monitored for 4 days after injection (see FIG. 1.18).

To validate the imaging data we performed histological analysis in which treated muscle tissue was removed after injection (cells in this example were labeled prior to injection with a green cell tracking fluorophore), fixed, and stained with hematoxylin and eosin. This sample was first placed in a fluorescence imaging chamber using green fluorescent protein filter cubes. Intense green fluorescence is seen corresponding to the green cell tracking dye in the MSCs (FIG. 1.19) The resulting histology slide shows very clear morphological differences between skeletal muscle (FIG. 1.7C; right) and the delivered cells (FIG. 1.7C; left). The fluorescence of the cell tracking dye is obvious when an adjacent slice is imaged with fluorescence (FIG. 1.7D). Although there was some damage during sample preparation causing the delivered cells to lose adherence to the muscle tissue, this confirms that the increase in imaging signal is due to cells. Interestingly, at 40× magnification, dark spots are present in MSCs, likely due to SiGNRs (FIG. 1.7E).

### Discussion

SiGNRs were used as a photoacoustic contrast agent to image MSCs implanted into rodent muscle. The silica coat played two important roles—it enhanced the photoacoustic signal of the GNRs⁴⁷ (FIG. 1.1) and increased uptake of the GNRs into the cell (FIG. 1.16). TEM evidence suggested that the SiGNRs were endocytosed into vesicles inside the MSCs (FIGS. 1.3 and 1.14). Optimal conditions (3 hours incubation at 0.07 nM) were found such that PA signal remained high, but with no negative impact on cell metabolism or proliferation (FIGS. 1.2, 1.4, and 1.5). To the best of our knowledge, this is the first example of in vivo photoacoustic MSC imaging. This approach allows real-time (5 frames per second) PA imaging with the B-mode ultrasound image offering clear anatomic features and the photoacoustic data showing cell specific content at sub-mm resolution.

Very good detection limits for both the contrast alone (0.03 nM) and MSCs (5,000 ex vivo; 90,000 in vivo) were measured and thus the sensitivity of this approach is suitable for imaging MSCs in vivo. Furthermore, the injection procedure (25 gauge catheter) caused very little trauma resulting in background photoacoustic signal (FIG. 1.6H, 1.6K, 1.6N). It is important to note that the number of cells used here is more than two orders of magnitude below what would be delivered clinically, and complements nicely the existing ultrasound infrastructure.⁴⁸

Previous reports have shown that surface charge, polymer coatings, and incubation concentration can affect the loading level of GNRs into cancer cell lines including MBT2,⁴⁹ HeLa,⁵° and HT29 cells.⁵¹ Values in these experiments range from 50-150,000 nanorods per cell.⁴⁹⁻⁵¹ This work with SiGNRs shows that a very high amount of contrast can be loaded into these cells (101,000±1,000 SiGNRs/MSC by ICP). For 30 μm diameter MSCs, this translates into 0.005% of the cell volume being occupied by SiGNRs or 12 nM. While this value is much higher than the concentration shown to induce toxicity (FIG. 1.2), this in vivo concentration is in vacuoles that likely prevent toxicity. Nevertheless, proliferation and metabolic screens indicated these MSCs behaved as non-labeled MSCs (FIG. 1.2). The pluripotency of the MSCs is retained as illustrated for osteogenic and adipogenic differentiation (FIG. 1.4). Furthermore, there is no unintended differentiation, which is a concern since nanoparticles can sometimes give rise to spontaneous osteogenesis.^{52, 53} More importantly, the relatively stable secretome suggests most cellular pathways are unaffected by SiGNRs and that any paracrine effects of MSC therapy will remain available to damaged tissue.⁵⁴

However, there are some important considerations to measuring SiGNR-labeled cells. Challenges inherent to PA imaging include light scatter, inaccuracies in reconstructions, frequency/signal changes due to volume modifications, tissue background, and attenuation of the excitation source. The signal may be especially reduced at deeper implant sites, even though some reports show PA depth penetration of several centimeters.^{23, 55} Although suitable for imaging cells in muscle tissue, applications in deeper areas may require the use of a photoacoustic catheter or endoscope, which are under construction in our lab. Also in preparation are more sophisticated analysis schemes to analyze the images on a pixel-by-pixel basis rather than with ROI analysis. We continuously monitored one treated animal; the cell bolus could be monitored for 4 days after injection (FIG. 1.18), but further work is needed to use PAI along for long term cell tracking due to the limitations mentioned above. In addition, the current generation of tunable lasers do not have extremely tight stability of power output—resolving the stability of laser power is critical to making reproducible photoacoustic measurements since PA signal directly correlates to intensity of the incident laser pulse. Future work will explore multispectral imaging. In the meantime, we use a feedback loop (FIG. 1.9) in which the laser output power is constantly monitored and the resulting output PA signal is normalized to the laser intensity.

Importantly, clinical adaptations of this work could only label a percentage of the cells, leaving the remainder free of contrast. The next generation of this contrast agent may have a larger aspect ratio to induce longer red-shifted resonances.⁵⁶ Finally, we will dope Gd³⁺ into the silica shell of the SiGNRs for T1 magnetic resonance imagine to complement the PA mode for long-term monitoring of implanted MSCs with greater depth of penetration. This work is an important next step in imaging stem cell delivery in real time and this report is the first example of stem cell quantitation with the photoacoustic modality. B-mode imaging for visualization of the delivery catheter and the in vivo environment is complemented nicely by PA-mode that specifically enhances MSC signal.

### CONCLUSION

SiGNRs were used as PA contrast agents to label MSCs. Cells were imaged ex vivo in an agarose phantom and in vivo after intra-muscular injection. Cell detection limits in vivo (100,000) were well below clinically relevant numbers. Imaging data was confirmed with histology. Proper cell loading conditions were selected such that metabolism, proliferation, and pluripotency were retained. Secretome analysis indicates that a wide variety of cytokines and chemokines were differentially expressed in the SiGNR-labeled MSCs, but 25 of the 26 proteins had expression levels with changes within one-fold of baseline. These data suggest that the therapeutic benefit of the MSCs will be retained despite the presence of contrast agent and the 0.2 s temporal resolution of the PA imaging technique can offer real time content on cell location and number.

### Materials and Methods.

Reagents. The following reagents were acquired and used as received: cetyltrimethylammonium bromide (CTAB; Sigma Aldrich), gold (III) chloride (Sigma Aldrich), sodium borohydride (Fluka), ascorbic acid (Sigma Aldrich), silver nitrate (Acros), 10 M sodium hydroxide (Sigma Aldrich), tetraethyl orthosilicate (TEOS, Acros), dimethylthiazolyl-diphenyltetrazolium (MTT; Biotium), phosphate buffered saline (PBS, Gibco), SP-DiOC18(3) cell tracking dye (Invitrogen), Oil Red O (Sigma Aldrich), Alizarin Red S (Sigma Aldrich), and agarose (Invitrogen). Millipore water (at 18 MOhm) was used. A Synergy 4 (Biotek) microplate reader was used for cell assays.

Gold Nanorod Synthesis. The GNRs were prepared via the seeded-growth mechanism previously described with slight modifications.^{56, 57} Briefly, gold seed was prepared by the addition of 5 mL 0.2 M CTAB to 5 mL of 0.005 M gold chloride in a scintillation vial. Then, 0.6 mL of 0.01 M NaBH₄ (previously chilled for ten minutes in an ice water bath) was quickly added and the mixture shaken for two minutes. The growth mixture was prepared with the following: 250 mL 0.2 M CTAB, 250 mL 0.001 M AuCl₃, and 12 mL 4 mM AgNO₃. This solution was yellow/brown, but became translucent upon addition of 3.5 mL of 0.0788 M ascorbic acid. Seed (0.6 mL) was then added and the solution became purple/brownish over 30 minutes. The GNRs were purified with four rounds of centrifugation and water washing at 16,000 rcf for 20 minutes and characterized with transmission electron microscopy (TEM), absorption spectroscopy, and dynamic light scattering (DLS; Malvern Zetasizer). A molar extinction coefficient from the literature of 3.1×10⁹ M⁻¹ cm⁻¹ at the peak resonance was used for GNRs with resonance near 660 nm.^{58, 59}

Silica Coating. SiGNRs were prepared by diluting stock GNRs to 2.2 nM in water (10 mL total volume) and treating with 100 μL of 0.1 N NaOH to achieve pH of ˜10. TEOS (6 μL) was added three times, 30 minutes apart and the reaction was allowed to proceed overnight.⁶⁰ The next day, SiGNRs were centrifuged at 6,000 rcf for 5 minutes, redissolved in water, and briefly sonicated to re-suspend.

Cell Culture. All experiments were done with MSCs between passage number 3 and 12 and used 3-6 replicate wells. Cells and media (including differentiation media) were acquired from Lonza. Unless otherwise noted, cells were plated at 5000 cells/cm² of culture plate area and loaded with nanoparticles 2-7 days after plating (˜80% confluence). Cells were counted after harvest and washing. The total number of cells required was dependent on the end application. We used large T225 flasks (25 mL volume) for the muscle implantation experiments, T75 flasks (10 mL volume) but 6 well plates (2 mL) for the loading optimization assays. To label MSCs with SiGNRs, we added SiGNRs to the culture flasks at a working concentration between 0.0 and 0.14 nM SiGNRs with incubation from 3 to 20 hours. Cells loaded with silica-free GNRs were treated identically to the SiGNRs. Cells were then washed thrice with PBS and removed from the flask with TripLE express (Invitrogen). Toxicity assays were performed by plating 10,000 MSCs/well in 96 well plates and loading SiGNRs in situ. Proliferation assays started with a 3000 cells/well with six replicate wells in 96 well plate.

Inductively Coupled Plasma (ICP). We used ICP to determine the amount of SiGNRs in MSCs. 50,000 MSCs were plated in each well of a 6 well plate and grown to near confluency. Three groups were used: MSCs with SiGNRs, MSCs with regular GNRs, and MSCs with no contrast agent. Cells with contrast agent were loaded with SiGNRs or GNRs with isomolar (0.07 nM) and isovolume (2 mL) conditions along with MSCs with no contrast agent. After 3 hours, the media of all wells was removed and cells washed three times with room temperature PBS and removed with trypsin. Cells were counted and transferred to 20% aqua regia in water to dissolve the SiGNRs. The samples were placed in a bath sonicator for 20 minutes to ensure completely dissolution of the cell. Gold ICP standard (Fluka) was used to construct a standard curve. The volume was brought to 5 ml and analyzed for the presence of gold ions with an IRIS Advantage/1000 Radial ICAP Spectrometer (Thermo Scientific). Standards were analyzed in duplicate and cells samples analyzed in triplicate with nearly 100,000 MSCs analyzed per sample.

Differentiation Experiments. Low passage number (<6) MSCs were used for differentiation experiments. Cells were loaded with SiGNRs as described above and the labeled cells were counted and plated as described below. Stained cells were imaged with a Leica light microscope.

The osteogenic protocol used 35 mm collagen-coated culture plates (World Precision Instruments) and 30,000 cells (loaded and unloaded with SiGNRs) per plate. The next day, standard media was replaced with osteogenic media (Lonza PT-3002). Control cells used standard media, and osteogenic media was supplemented with dexamethasone, ascorbate, and b-glycophophate. The media for both control and labeled cells was changed every 2-3 days. After 24 days, cells were fixed with 70% ethanol on ice for one hour and then stained with 2% Alizarin Red in water (pH 4.2; freshly filtered) for 7 minutes followed by water washes until no excess stain was removed. The degree of osteogenesis was quantitated by dissolving the colored complex in 10% acetic acid and measuring A402.

In the adipogenic protocol, 80,000 loaded and unloaded cells were seeded in a 12 well plate and grown for 7 days until they were over-confluent. Cells in the induced population were subjected to three rounds of three-day growth in induction media (Lonza PT-3004) followed by 1-3 intervals in maintenance media. Adipogenic induction media contained recombinant insulin, dexamethasone, indomethacin, 3-isobutyl-1-methyl-xanthine, and gentamicin. Adipogenic maintenance media contained only insulin and gentamicin. Control cells were incubated only in maintenance media. One week after the final round of induction, cells contained a large number of microscopic lipid vacuoles. The MSCs were fixed in 10% formalin for 45 minutes and washed with water and then 60% isopropyl alcohol. Oil red O was used to stain the adipogenic cells. To prepare this stain, 18 mL water was added to 27 mL of 3 mg/mL Oil red O in isopropyl alcohol. After ten minutes the solution was filtered and added to the fixed cells for five minutes followed by water wash. Cells were counterstained with hematoxylin for 2 minutes.

Cytokine Expression. Cells with and without SiGNRs were plated at 20,000/cm² in a 12 well plate and cultured for two days. The media was then exchanged and allowed to stand for 24 hours. That media from positive and control cells was then removed along with media without cells that had been in the incubator for the same amount of time. Secretome analysis was performed with a bead-based assay (Luminex) by a commercial operator (Rules Based Medicine; Austin, Tex.)⁴⁶. Assays used 8 calibrations standards per protein and three controls. The antibodies for capture and detection were directed against all isotypes.⁶¹ Every protein was measured with a redundancy of 50 beads.

PA Imaging. Photoacoustic imaging was performed with a LAZR commercial instrument (Visualsonics) equipped with a 21 MHz-centered transducer and described previously.⁶² The system uses a flashlamp pumped Q-switched Nd:YAG laser with optical parametric oscillator and second harmonic generator operating at 20 Hz between 680 and 970 nm with a 1 nm step size and as pulse of 4-6 ns. The peak energy is 45±5 mJ at 20 Hz at source. The spot size is 1 mm×24 mm and the full field of view is 14-23 mm wide. Acquisition rate is 5 frames per second. Imaging the SiGNRs was originally done in agarose phantoms. These were prepared by first boiling 1 mg/mL agarose in degassed distilled water and pouring 20 mL of the hot mixture into a 10 cm petri dish and allowing it to cool briefly. Once sufficient surface tension had been achieved due to cooling (˜2-3 minutes), we added 2.5 cm sections of polyethylene tubing (Intramedic, PE190, outer diameter 1.70 mm), which floated on top of the agarose and served as a mold. After complete cooling, the tube molds were removed to leave an indentation in the cooled gel. These voids were filled with either contrast agents or MSCs (15 μL) mixed with 15 μL 50% warm 1 mg/mL agarose. The phantom was sealed with a final 2-4 mm of agarose. Typical imaging conditions include 100% power, 50 dB gain, 21 MHz frequency, and 680 nm excitation. The laser output was monitored externally on the animal bed with a Gentec-eo power meter with sensor 1 cm from end of PA transducer as well as internal power sampling.

Animal Studies. Female nu/nu mice (6-16 weeks old) were used in this study in triplicate at each data point. All animal work was conducted in accordance with the Administrative Panel on Laboratory Animal Care at Stanford University. Prior to imaging, mice were anesthetized with 2% isofluorane in house oxygen at 2 L/min and confirmed with tail pinch. MSCs pellets were resuspended in 40 μL of PBS and mixed with an equivalent volume of ice-cold matrigel. This 80-μL cell-containing bolus was loaded into a 0.5 mL insulin syringe (20 μL dead volume) and allowed to come to room temperature prior to delivery to increase the viscosity of the material. Delivery used the syringe in tandem with a 25 gauge winged infusion set. For histology confirmation, cells were labeled with a lipophilic carbocyanine cell tracing dye (SP-DiOC18(3)) prior to injection. This protocol used a 1 μM working solution for 5 minutes in the cell culture incubator on adherent cells followed by 15 minutes in the cold room. Cells were then removed with trypsin for injection.

Histology. Tissue sections were removed and immediately placed in 10% buffered formalin (Fisher) for two days and then transferred to 30% sucrose in PBS. Sections were then placed in optimal cutting temperature (OCT) media and froze for ten seconds in a bath of isopentane that was immersed in a bath of liquid nitrogen. Tissue sections (6 μm) were sliced and placed on charged slides and imaged with an automated histology slide reading tool (Nanozoomer).

Microscopy. All transmission electron microscopy (TEM) and energy-dispersive x-ray spectroscopy (EDS) was performed with a Tecnai G2 X-Twin (FEI Co.) instrument operating at 200 kV. After loading with SiNPs, MSCs were washed three times with PBS, removed from the flask with tryspin, washed with media and saline using 5 min of 1000×g centrifugation to create the pellet and prepared for TEM as described previously.⁶³ Briefly, cells were transferred to a 2:1:1 solution of 0.2 M sodium cacodylate buffer/10% glutaraldehyde/8% paraformaldehye (EMSdiasum). Samples were then stored at 4° C. before being stained en bloc with osmium tetroxide. After 2 h, samples were rinsed with deionized water and stained with uranyl acetate overnight. Samples were dehydrated in progressively higher concentrations of ethanol in water: 50, 70, 95, and 100%. Samples were further dehydrated using propylene oxide and embedded in Embed 812 epoxy resin (EMSdiasum). Thin sections (500 nm) were cut using a Leica Ultracut S microtome and placed on a 200-mesh bare copper grid (Ted Pella). Fluorescence microscopy was performed with a Leica Confocal System and white light microscopy utilized an Axiovert 25 steromicroscope (Zeiss) fitted with a CCD detector and MR Grab software.

Data Analysis. The limit of detection (LOD) was defined as signal detectable three standard deviations above the mean signal of the blank. Images were saved as RGB TIFF files and analyzed with Image J software.⁶⁴ In phantoms, PA signal intensity was measured by region of interest (ROI) analysis and signal was defined as the PA-mode contrast generated by the SiGNR inclusion. Background was defined as the PA-mode ultrasound contrast generated by the agarose gel surrounding the inclusion. In animals, we created contrast enhanced images by subtracting the pre and post-injection PA images and taking the mean in the ROI. We then performed a dynamic threshold on the image. We used the ROI mean for the lower end and the three times that mean on the upper end. These pixels were then assigned to a green look up table, overlaid with the original post-injection (red) image to illustrate enhanced pixels by making the resulting positive pixels green or yellow (green+red). The animal LOD was determined as above using the mean ROI intensity at the injection site and sham injection. Analysis of secretome data divided the mean value of the SiGNR cells by control cells (no SiGNRs). Statistical analysis of secretome data used a two tailed t-test with 49 degrees of freedom (t=1.960) with the assumption that the coefficient of variation applied to both SiGNR and control samples. P-values were calculated from the experimental t values using Microsoft Excel command “T.DIST”.

References, each of which is incorporated herein by reference
1. LaBarge, M. A.; Blau, H. M., Biological Progression from Adult Bone Marrow to Mononucleate Muscle Stem Cell to Multinucleate Muscle Fiber in Response to Injury. Cell 2002, 111, 589-601.
2. Meregalli, M.; Farini, A.; Parolini, D.; Maciotta, S.; Torrente, Y., Stem Cell Therapies to Treat Muscular Dystrophy: Progress to Date. BioDrugs 2010, 24, 237-47.
3. Sohn, R. L.; Gussoni, E., Stem Cell Therapy for Muscular Dystrophy. Exp. Op. Bio. Ther. 2004, 4, 1-9.
4. Corbel, S. Y.; Lee, A.; Yi, L.; Duenas, J.; Brazelton, T. R.; Blau, H. M.; Rossi, F. M. V., Contribution of Hematopoietic Stem Cells to Skeletal Muscle. Nat. Med. 2003, 9, 1528-1532.
5. Torrente, Y.; Belicchi, M.; Sampaolesi, M.; Pisati, F.; Meregalli, M.; D Antona, G.; Tonlorenzi, R.; Porretti, L.; Gavina, M.; Mamchaoui, K., Human Circulating Ac133̂+ Stem Cells Restore Dystrophin Expression and Ameliorate Function in Dystrophic Skeletal Muscle. J. Clin. Invest. 2004, 114, 182-195.
6. Price, F.; Kuroda, K.; Rudnicki, M., Stem Cell Based Therapies to Treat Muscular Dystrophy. Biochim. Biophys. Acta (BBA) 2007, 1772, 272-283.
7. Shabbir, A.; Zisa, D.; Leiker, M.; Johnston, C.; Lin, H.; Lee, T., Muscular Dystrophy Therapy by Non-Autologous Mesenchymal Stem Cells: Muscle Regeneration without Immunosuppression and Inflammation. Transplantation 2009, 87, 1275.
8. Montarras, D.; Morgan, J.; Collins, C.; Relaix, F.; Zaffran, S.; Cumano, A.; Partridge, T.; Buckingham, M., Direct Isolation of Satellite Cells for Skeletal Muscle Regeneration. Science 2005, 309, 2064.
9. Markert, C. D.; Atala, A.; Cann, J. K.; Christ, G.; Furth, M.; Ambrosio, F.; Childers, M. K., Mesenchymal Stem Cells: Emerging Therapy for Duchenne Muscular Dystrophy. PM R 2009, 1, 547-59.
10. Liechty, K. W.; MacKenzie, T. C.; Shaaban, A. F.; Radu, A.; Moseley, A.; Deans, R.; Marshak, D. R.; Flake, A. W., Human Mesenchymal Stem Cells Engraft and Demonstrate Site-Specific Differentiation after in Utero Transplantation in Sheep. Nat. Med. 2000, 6, 1282-1286.
11. Ichim, T. E.; Alexandrescu, D. T.; Solano, F.; Lara, F.; Campion, R. D. N.; Paris, E.; Woods, E. J.; Murphy, M. P.; Dasanu, C. A.; Patel, A. N., Mesenchymal Stem Cells as Anti-Inflammatories: Implications for Treatment of Duchenne Muscular Dystrophy. Cellular immunology 2010, 260, 75-82.
12. Krampera, M.; Pizzolo, G.; Aprili, G.; Franchini, M., Mesenchymal Stem Cells for Bone, Cartilage, Tendon and Skeletal Muscle Repair. Bone 2006, 39, 678-683.
13. Jiang, Y.; Jahagirdar, B. N.; Reinhardt, R. L.; Schwartz, R. E.; Keene, C. D.; Ortiz-Gonzalez, X. R.; Reyes, M.; Lenvik, T.; Lund, T.; Blackstad, M., Pluripotency of Mesenchymal Stem Cells Derived from Adult Marrow. Nature 2002, 418, 41-9.
14. Segers, V. F.; Lee, R. T., Stem-Cell Therapy for Cardiac Disease. Nature. 2008, 451, 937-42.
15. de Vries, I. J.; Lesterhuis, W. J.; Barentsz, J. O.; Verdijk, P.; van Krieken, J. H.; Boerman, O. C.; Oyen, W. J.; Bonenkamp, J. J.; Boezeman, J. B.; Adema, G. J.; Bulte, J. W.; Scheenen, T. W.; Punt, C. J.; Heerschap, A.; Figdor, C. G., Magnetic Resonance Tracking of Dendritic Cells in Melanoma Patients for Monitoring of Cellular Therapy. Nat. Biotechnol. 2005, 23, 1407-13.
16. Bulte, J. W. M., In Vivo Mri Cell Tracking: Clinical Studies. Am. J. Roentgen. 2009, 193, 314.
17. Kircher, M. F.; Gambhir, S. S.; Grimm, J., Noninvasive Cell-Tracking Methods. Nat. Rev. Clin. Pract. 2011, 8, 677-688.
18. Modo, M.; Cash, D.; Mellodew, K.; Williams, S. C. R.; Fraser, S. E.; Meade, T. J.; Price, J.; Hodges, H., Tracking Transplanted Stem Cell Migration Using Bifunctional, Contrast Agent-Enhanced, Magnetic Resonance Imaging. Neuroimage 2002, 17, 803-811.
19. Kraitchman, D. L.; Heldman, A. W.; Atalar, E.; Amado, L. C.; Martin, B. J.; Pittenger, M. F.; Hare, J. M.; Bulte, J. W. M., In Vivo Magnetic Resonance Imaging of Mesenchymal Stem Cells in Myocardial Infarction. Circulation 2003, 107, 2290.
20. Hill, J. M.; Dick, A. J.; Raman, V. K.; Thompson, R. B.; Yu, Z. X.; Hinds, K. A.; Pessanha, B. S. S.; Guttman, M. A.; Varney, T. R.; Martin, B. J., Serial Cardiac Magnetic Resonance Imaging of Injected Mesenchymal Stem Cells. Circulation 2003, 108, 1009.
21. Foster Gareau, P.; Heyn, C.; Alejski, A.; Rutt, B. K., Imaging Single Mammalian Cells with a 1.5 T Clinical Mri Scanner. Magn. Reson. Med. 2003, 49, 968-971.
22. Shapiro, E. M.; Sharer, K.; Skrtic, S.; Koretsky, A. P., In Vivo Detection of Single Cells by Mri. Magn. Reson. Med. 2006, 55, 242-9.
23. Xu, M.; Wang, L. V., Photoacoustic Imaging in Biomedicine. Rev. Sci. Instr. 2006, 77, 041101.
24. Razansky, D.; Distel, M.; Vinegoni, C.; Ma, R.; Perrimon, N.; Koster, R. W.; Ntziachristos, V., Multispectral Opto-Acoustic Tomography of Deep-Seated Fluorescent Proteins in Vivo. Nature Photonics 2009, 3, 412-417.
25. Zerda, A.; Liu, Z.; Bodapati, S.; Teed, R.; Vaithilingam, S.; Khuri-Yakub, B. T.; Chen, X.; Dai, H.; Gambhir, S. S., Ultrahigh Sensitivity Carbon Nanotube Agents for Photoacoustic Molecular Imaging in Living Mice. Nano Lett. 2010, 10, 2168-2172.
26. Razansky, D.; Vinegoni, C.; Ntziachristos, V., Multispectral Photoacoustic Imaging of Fluorochromes in Small Animals. Optics letters 2007, 32, 2891-2893.
27. KÃ¶stli, K. P.; Beard, P. C., Two-Dimensional Photoacoustic Imaging by Use of Fourier-Transform Image Reconstruction and a Detector with an Anisotropic Response. Applied optics 2003, 42, 1899-1908.
28. Kim, J. W.; Galanzha, E. I.; Shashkov, E. V.; Moon, H. M.; Zharov, V. P., Golden Carbon Nanotubes as Multimodal Photoacoustic and Photothermal High-Contrast Molecular Agents. Nat. Nanotechnol. 2009, 4, 688-694.
29. Lu, W.; Huang, Q.; Ku, G.; Wen, X.; Zhou, M.; Guzatov, D.; Brecht, P.; Su, R.; Oraevsky, A.; Wang, L. V., Photoacoustic Imaging of Living Mouse Brain Vasculature Using Hollow Gold Nanospheres. Biomaterials 2010, 31, 2617-2626.
30. Kruger, R. A.; Lam, R. B.; Reinecke, D. R.; Del Rio, S. P.; Doyle, R. P., Photoacoustic Angiography of The Breast. Med. Phys 2010, 37, 6096-6100.
31. Wang, X.; Xie, X.; Ku, G.; Wang, L. V.; Stoica, G., Noninvasive Imaging of Hemoglobin Concentration and Oxygenation in The Rat Brain Using High-Resolution Photoacoustic Tomography. J. Biomed. Opt. 2006, 11, 024015.
32. Levi, J.; Kothapalli, S. R.; Ma, T. J.; Hartman, K.; Khuri-Yakub, B. T.; Gambhir, S. S., Design, Synthesis, and Imaging of an Activatable Photoacoustic Probe. J Am Chem Soc 2010, 132, 11264-9.
33. De la Zerda, A.; Zavaleta, C.; Keren, S.; Vaithilingam, S.; Bodapati, S.; Liu, Z.; Levi, J.; Smith Bryan, R.; Ma, T.-J.; Oralkan, O.; Cheng, Z.; Chen, X.; Dai, H.; Khuri-Yakub Butrus, T.; Gambhir Sanjiv, S., Carbon Nanotubes as Photoacoustic Molecular Imaging Agents in Living Mice. Nat. Nanotechnol. 2008, 3, 557-62.
34. Tong, L.; Wei, Q.; Wei, A.; Cheng, J. X., Gold Nanorods as Contrast Agents for Biological Imaging: Optical Properties, Surface Conjugation and Photothermal Effects. Photochem Photobiol. 2009, 85, 21-32.
35. Chen, Y. S.; Frey, W.; Kim, S.; Kruizing a, P.; Homan, K.; Emelianov, S., Silica-Coated Gold Nanorods as Photoacoustic Signal Nanoamplifiers. Nano Lett 2011, 11, 348-54.
36. Slowing, I.; Trewyn, B. G.; Victor, S. Y. L., Effect of Surface Functionalization of Mcm-41-Type Mesoporous Silica Nanoparticles on The Endocytosis by Human Cancer Cells. J. Am. Chem. Soc 2006, 128, 14792-14793.
37. Chen, L. C.; Wei, C. W.; Souris, J. S.; Cheng, S. H.; Chen, C. T.; Yang, C. S.; Li, P. C.; Lo, L. W., Enhanced Photoacoustic Stability of Gold Nanorods by Silica Matrix Confinement. J. Biomed. Opt. 2010, 15, 016010.
38. Hackley, V. A.; Clogston, J. D., Measuring The Hydrodynamic Size of Nanoparticles in Aqueous Media Using Batch-Mode Dynamic Light Scattering. Meth. Mol. Biol. 2011, 697, 35.
39. Labbaf, S.; Tsigkou, O.; Muller, K. H.; Stevens, M. M.; Porter, A. E.; Jones, J. R., Spherical Bioactive Glass Particles and Their Interaction with Human Mesenchymal Stem Cells in Vitro. Biomaterials 2010.
40. Chung, T. H.; Wu, S. H.; Yao, M.; Lu, C. W.; Lin, Y. S.; Hung, Y.; Mou, C. Y.; Chen, Y. C.; Huang, D. M., The Effect of Surface Charge on The Uptake and Biological Function of Mesoporous Silica Nanoparticles in 3t3-L1 Cells and Human Mesenchymal Stem Cells. Biomaterials 2007, 28, 2959-2966.
41. Jokerst, J. V.; Miao, Z.; Zavaleta, C.; Cheng, Z.; Gambhir, S. S., Affibody-Functionalized Gold-Silica Nanoparticles for Raman Molecular Imaging of The Epidermal Growth Factor Receptor. Small 2011, 7, 625-33.
42. Rides, L. M.; Nam, S. Y.; Sokolov, K.; Emelianov, S. Y.; Suggs, L. J., Function of Mesenchymal Stem Cells Following Loading of Gold Nanotracers. Int. J. Nanomed. 2011, 6, 407-16.
43. Hsiao, J. K.; Tsai, C. P.; Chung, T. H.; Hung, Y.; Yao, M.; Liu, H. M.; Mou, C. Y.; Yang, C. S.; Chen, Y. C.; Huang, D. M., Mesoporous Silica Nanoparticles as a Delivery System of Gadolinium for Effective Human Stem Cell Tracking. Small 2008, 4, 1445-1452.
44. Amaral, M.; Costa, M.; Lopes, M.; Silva, R.; Santos, J.; Fernandes, M., Si3n4-Bioglass Composites Stimulate The Proliferation of Mg63 Osteoblast-Like Cells and Support The Osteogenic Differentiation of Human Bone Marrow Cells. Biomaterials 2002, 23, 4897-4906.
45. Aggarwal, S.; Pittenger, M. F., Human Mesenchymal Stem Cells Modulate Allogeneic Immune Cell Responses. Blood 2005, 105, 1815-1822.
46. Dunbar, S. A., Applications of Luminex Xmap Technology for Rapid, High-Throughput Multiplexed Nucleic Acid Detection. Clin. Chim. Acta. 2006, 363, 71-82.
47. Chen, L. C.; Wei, C. W.; Souris, J. S.; Cheng, S. H.; Chen, C. T.; Yang, C. S.; Li, P. C.; Lo, L. W., Enhanced Photoacoustic Stability of Gold Nanorods by Silica Matrix Confinement. J. Biomed. Opt. 2010, 15, 016010.
48. Sampaolesi, M.; Blot, S.; D'Antona, G.; Granger, N.; Tonlorenzi, R.; Innocenzi, A.; Mognol, P.; Thibaud, J. L.; Galvez, B. G.; Barthelemy, I., Mesoangioblast Stem Cells Ameliorate Muscle Function in Dystrophic Dogs. Nature 2006, 444, 574-579.
49. Li, P. C.; Wei, C. W.; Liao, C. K.; Chen, C. D.; Pao, K. C.; Wang, C. R. C.; Wu, Y. N.; Shieh, D. B., Photoacoustic Imaging of Multiple Targets Using Gold Nanorods. IEEE Ultrason. Ferroelec. Freq. 2007, 54, 1642-1647.
50. Hauck, T. S.; Ghazani, A. A.; Chan, W. C. W., Assessing The Effect of Surface Chemistry on Gold Nanorod Uptake, Toxicity, and Gene Expression in Mammalian Cells. Small 2008, 4, 153-159.
51. Alkilany, A. M.; Nagaria, P. K.; Hexyl, C. R.; Shaw, T. J.; Murphy, C. J.; Wyatt, M. D., Cellular Uptake and Cytotoxicity of Gold Nanorods: Molecular Origin of Cytotoxicity and Surface Effects. Small 2009, 5, 701-708.
52. Gonzalez McQuire, R.; Green, D. W.; Partridge, K. A.; Oreffo, R. O. C.; Mann, S.; Davis, S. A., Coating of Human Mesenchymal Cells in 3d Culture with Bioinorganic Nanoparticles Promotes Osteoblastic Differentiation and Gene Transfection. Advanced materials 2007, 19, 2236-2240.
53. Huang, D. M.; Chung, T. H.; Hung, Y.; Lu, F.; Wu, S. H.; Mou, C. Y.; Yao, M.; Chen, Y. C., Internalization of Mesoporous Silica Nanoparticles Induces Transient but Not Sufficient Osteogenic Signals in Human Mesenchymal Stem Cells. Toxicol. Appl. Pharmacol. 2008, 231, 208-215.
54. Discher, D. E.; Mooney, D. J.; Zandstra, P. W., Growth Factors, Matrices, and Forces Combine and Control Stem Cells. Science 2009, 324, 1673-1677.
55. Didychuk, C. L.; Ephrat, P.; Chamson-Reig, A.; Jacques, S. L.; Carson, J. J. L., Depth of Photothermal Conversion of Gold Nanorods Embedded in a Tissue-Like Phantom. Nanotechnology 2009, 20, 195102.
56. Nikoobakht, B.; El-Sayed, M., Preparation and Growth Mechanism of Gold Nanorods (Nrs) Using Seed-Mediated Growth Method. Chem. Mater 2003, 15, 1957-1962.
57. Wei, A.; Leonov, A. P.; Wei, Q., Gold Nanorods: Multifunctional Agents for Cancer Imaging and Therapy. Meth Mol Biol 2010, 624, 119-30.
58. Liao, H.; Hafner, J. H., Gold Nanorod Bioconjugates. Chemistry of materials 2005, 17, 4636-4641.
59. Orendorff, C. J.; Murphy, C. J., Quantitation of Metal Content in The Silver-Assisted Growth of Gold Nanorods. The Journal of Physical Chemistry B 2006, 110, 3990-3994.
60. Gorelikov, I.; Matsuura, N., Single-Step Coating of Mesoporous Silica on Cetyltrimethyl Ammonium Bromide-Capped Nanoparticles. Nano Lett. 2008, 8, 369-373.
61. Craig-Schapiro, R.; Kuhn, M.; Xiong, C.; Pickering, E. H.; Liu, J.; Misko, T. P.; Perrin, R. J.; Bales, K. R.; Soares, H.; Fagan, A. M., Multiplexed Immunoassay Panel Identifies Novel CSF Biomarkers for Alzheimer's Disease Diagnosis and Prognosis. PLoS One 2011, 6, e18850.
62. Needles, A.; Heinmiller, A.; Ephrat, P.; Bilan-Tracey, C.; Trujillo, A.; Theodoropoulos, C.; Hirson, D.; Foster, F. In Development of a Combined Photoacoustic Micro-Ultrasound System for Estimating Blood Oxygenation, IEEE: 2010; pp 390-393.
63. Thakor, A. S.; Paulmurugan, R.; Kempen P.; Zavaleta, C.; Sinclair, R.; Massoud, T. F.; Gambhir, S. S., Oxidative Stress Mediates The Effects of Raman Active Gold Nanoparticles in Human Cells. Small 2011, 7, 126-136.
64. Abramoff, M. D.; Magalhaes, P. J.; Ram, S. J., Image Processing with ImageJ. Biophoton. Intl. 2004, 11, 36-42.

### Example 2

### Brief Introduction

Imaging monitors the efficacy of cardiac regenerative medicine by reporting the viability, location, and number of implanted stem cells. Strategies utilizing positron emission tomography (PET), magnetic resonance imaging (MRI), and other approaches have been reported. Here, we describe a sub-micron, silica-based contrast agent with concurrent fluorescent, ultrasound (US), and MRI signaling capacity. This single agent can be used for cell sorting (fluorescence), real-time guided cell implantation (US), and high-resolution, long-term (2 weeks) serial monitoring (MRI and US). The agent increases the B-mode US and MRI contrast of labeled mesenchymal stem cells (MSCs) up to 700% and 200%, respectively, with increased contrast persisting up to thirteen days post loading (p<0.05) and compatibility with both pre-clinical and clinical imaging equipment. The agent had no significant (p<0.05) impact on MSC cell metabolic activity or proliferation. Electron microscopy and US imaging suggest that the mechanism of action is in vivo aggregation of the 300 nm SiNPs into larger silica frameworks that modulate US backscatter. The detection limit in cardiac tissue was 250,000 MSCs via MRI and 70,000 via US. To the best of our knowledge, this is the first application of US-guided cell tracking and the first report of such a triple modality optical/US/MRI strategy that has important advantages for cost-effective clinical translation.

### Introduction

Morbidity and mortality due to ischemic heart disease continues to be a significant challenge in cardiovascular medicine. The therapeutic role of stem cells including cardiac stem cells (CSCs)¹, cardiac progenitor cells (CPCs)², embryonic stem cells (ESCs)³, and mesenchymal stem cells (MSCs)⁴ in cardiovascular disease (CVD) including myocardial infarction has recently been detailed in a number of animal studies and human clinical trials⁵⁻⁷. The therapeutic role of MSCs is currently understood to be via the release of therapeutic paracrine factors (cytokines, growth factors, chemokines) that enhance angiogenesis, reduce inflammation, and encourage proliferation of endogenous progenitor cells^{4,8-10}. MSCs are characterized by a very low incidence of adverse events with benefits including increased ejection fraction, reduced ventricular tachycardia, and reverse remodeling in humans¹¹. While there are several clinical trials in progress or completed to study stem cell therapy on CVD patients, safe, consistent, and effective results have yet to be demonstrated^{5,7,8,12}.

Specific limitations to stem cell therapy (SCT) include cell death due to ischemia, anoikis, and immune response, contamination by undifferentiated cells, and cell delivery into fibrotic tissue¹³. In one of the first human examples of SCT, cells were mis-injected in 50% of patients^{14,15}. In that study, the injection needle was positioned under ultrasound guidance by an experienced surgeon, but real time cell imaging was not performed and the poor injection rates were not identified until post-procedure MRI studies. Importantly, this work was in the lymph nodes, which is more straightforward region to inject than cardiac tissue. It is unclear what role poor delivery places in the poor survival in cardiac SCT.

Imaging is a fundamental tool to improve SCT and can assist with proper delivery of cells and monitor the short-term and long-term fate of delivered cells¹⁶. Such imaging is critical to determine the location(s) of cells not only during the transplant event, but also cell number, differentiation, and redistribution after implant. There are currently two approaches to stem cell tracking: 1) labeling the cells with a reporter gene or 2) tagging the cell with a contrast agent. Reporter gene-based techniques include intravital microscopy, in vivo confocal microscopy, bioluminescence, and positron emission tomography (PET). Although optical techniques offer very low cell detection limits and high spatial resolution they require substrates and reporter genes that are inconsistent with clinical translation and have depth penetrations too small for human use. PET imaging with reporter genes¹⁷ or exogenous agents (e.g. ⁶⁴Cu-PTSM)¹⁸ offer deep tissue imaging and quantitative analysis, but is a relatively expensive process and is incompatible with image-guided implantation.

Contrast agents for SCT are almost exclusively used with MRI. This modality (along with PET) is the most advanced technique for tracking stem cells including MSCs^{14, 19-24}. Tracking cells with MRI was first reported nearly a decade ago and current technology is capable of single cell imaging^{23,25}. Typical MRI contrast agents include superparamagnetic materials for T2 contrast^{26,27}; T1 contrast agents include gadolinium-based agents²⁸. While MRI has excellent resolution and soft tissue contrast with detection limits of 10-20 cells/voxel it has temporal resolution of tens of minutes and cannot currently be used in real time to guide the implantation of stem cells.

Ultrasound (US) is an very promising technology for cell tracking due to its broad access, high resolution, low cost, and high depth penetration²⁹. Unlike PET and MRI, US facilitates the real-time guidance of stem cell implantation in cardiac and abdominal applications. Ultrasound is especially promising for cardiac applications because of the ease and broad clinical acceptance of echocardiography. By allowing the physician to image cells during the initial placement, greater accumulation at the diseased site may be achieved. Although the catheter position is easily monitored via angiography and ultrasound, proper catheter position in no way ensures sufficient delivery and immobilization of cells at the desired location(s)^{14,29}. Unfortunately, the use of US for cell tracking is challenged by a lack of effective imaging agents³⁰. Although microbubbles have been used for vascular applications, their large size and composition prevents intra-cellular labeling, which is critical for cell implantation³¹. Microbubbles also fail to produce contrast beyond 30 minutes, which is too short for a typical cell tracking study. To address this limitation, we studied recent reports detailing sub-micron ultrasound contrast agents and hypothesized that they could be tailored to include a fluorescent and MRI reporter and deployed for SCT^{32,33}. Silica nanoparticles were particularly attractive because solid particles have a greater signaling potential than liquid particles³⁴.

In this Example, we report a fundamentally new approach to ultrasound imaging based on nanometer-scale, fluorescent, MRI-active, silica nanoparticles (SiNPs). After systemic characterization of their toxicity, cellular distribution, and stability, we use them to image MSCs via US in animal subjects after intracardiac implant. These probes offer intense, stable, real time, and multimodal signal in B-mode and contrast mode US as well as T1-shortening capabilities via Gd³⁺ doping. To the best of our knowledge, this is the first example of stem cell tracking with ultrasound and this approach offers a convenient and facile method to monitor cellular implantation in living subjects and monitor their progression.

### Materials and Methods

### Particle Synthesis

SiNPs were synthesized through a modified Stober synthesis^{35,36}. First, an organosilane conjugate of fluorescein isothiocyanate (FITC; Aldrich) was created by mixing 5.25 mg FITC Isomer with 73 μL of (3-aminopropyl)-triethoxysilane (APTMS; Alfa Aesar) in 1 mL ethanol (Gold Shield) overnight. The product was confirmed by mass spectrometry. The next day, 25 mL ethanol, 1.5 mL 30% ammonium hydroxide (EMD), and 1.5 mL distilled, deionized (DDI) water were added to a small Erlenmeyer flask with a stir bar at 500 RPM with 100 μL of the silanized FITC product. The contents were brought to 0.5 mg/mL GdCl₃ (Sigma Aldrich) and then two 1 mL aliquots of tetraethylorthosilicate (TEOS; Acros) were added in rapid succession. The mixture turned from a translucent yellow color to opaque over ˜2 hours and the reaction was allowed to proceed overnight. The size of the resulting nanomaterial was measured by dynamic light scattering (DLS; Zetasizer-90; Malvern) by diluting 15 μL of the reaction mixture to 1.5 mL of water. DLS parameters included the silica refractive index of 1.59 with absorbance of 0.45. Standard water values were used for the diluents. After SiNP growth was completed, the particles were pelleted at 6000 RPM (4629 RCF) on an Eppendorf 5804 centrifuge for 10 min and washed three times with ethanol and once with water. To remove any remaining free FITC or Gd³⁺, the particles were dialyzed overnight versus constantly refreshed water. Concentration values were determined by dehydrating known volumes of the material overnight in a 140° C. oven and measuring the resulting mass. The reproducibility of four batches was compared.

### Inductively Coupled Plasma (ICP) Studies

Three studies were performed using ICP. The first measured the amount of Gd³⁺ per SiNP via three runs each of 50, 100, and 200 μL of SiNPs. The second examined the stability of the Gd/Si system. Here, SiNPs (200 μL at 10 mg/mL) were added to 200 μL of mouse serum or water at 37° C. for 2 or 24 hours in triplicate. In both cases, the SiNPs were pelleted by centrifugation and supernatant retained for analysis of Gd³⁺ content. The SiNP pellet was dissolved with 1 mL 10 N NaOH with 40 minutes sonication, neutralized with 1 mL concentrated nitric acid, and diluted to 5 mL with 5% nitric acid. The supernatant was similarly dissolved in 5% nitric acid. The samples were analyzed with an ICAP 6300 system (Thermo Scientific) using 10 and 100 ppm solutions of Gd³⁺ (ICP standard grade; Fluka) in nitric acid as calibrations and standards. The number of SiNPs per volume was determined using the density of silica assuming a sphere with a size of 300 nm and the reported density of silica³⁷ for a molecular weight of 1.7×10¹⁰.

The third study determined the number of SiNPs per MSC. The number of cellular SiNPs were determined by measuring cellular Gd³⁺ and converting the ratio of Gd³⁺:SiNP as noted above. Cells were prepared similarly, using strong base to dissolve SiNPs. ICP analysis of cell culture media employed centrifugation to remove denatured proteins after adjusting to low pH.

### Optical Equipment

Flow cytometry was performed on a FACSCalibur (Becton Dickinson) with 5,000-10,000 cells collected per analysis. A Synergy 4 (Biotek) microplate reader was used for SiNP fluorescence characterization and cell proliferation/toxicity studies. For SiNP fluorescence, FITC filters cubes were used (485/20 nm excitation; 528/20 emission) with 35% detector sensitivity, and Xenon flash source.

### Cell Culture and Labeling

The MSCs and MSC growth media (PT-3001; Lonza) were used between passage 2 and 20. Cells were passaged when they reached 80% confluence with TripLE Express (Invitrogen) with approximately 3-7 days between each passage. Labeling with SiNPs was done without any exogenous transfection agents. SiNPs were added to media and allowed to incubate for 1 to 18 hours. The adherent cells were washed three times with phosphate buffered saline (PBS) prior to removal from the flask. Cells were re-suspended in PBS prior to flow cytometry analysis, ultrasound analysis, microscopy at 10×, or other analysis. Cell proliferation was studied after transfecting cells with SiNP using 3-(4,5-dimethylthiazol-2-yl)-2,5-diphenyltetrazolium bromide (MTT; Biotium).

### Differentiation Experiments

Low passage number (<6) MSCs were used for differentiation experiments and done at least in duplicate. Cells were loaded with SiNPs as described above and the labeled cells were counted and plated as described below. Stained cells were imaged with a Leica light microscope.

The osteogenic protocol used 35 mm collagen-coated culture plates (World Precision Instruments) and 30,000 cells (loaded and unloaded with SiNPs) per plate. The next day, standard media was replaced with osteogenic media (Lonza PT-3002) supplemented with dexamethasone, ascorbate, and b-glycophophate. Control cells used standard media. The media for both control and labeled cells was changed every 2-3 days. After 24 days, cells were fixed with 70% ethanol on ice for one hour and then stained with 2% Alizarin Red in water (pH 4.2; freshly filtered) for 7 minutes followed by water washes until no excess stain was removed. Dissolving the colored complex in 10% acetic acid and measuring the optical density at 402 nm quantitated the degree of osteogenesis.

In the adipogenic protocol, 80,000 SiNP-loaded and unloaded cells were seeded in a 12 well plate and grown for 7 days until they were over-confluent. Cells in the induced population were subjected to three rounds of three-day growth in induction media (Lonza PT-3004) followed by 1-3 intervals in maintenance media. Adipogenic induction media contained recombinant insulin, dexamethasone, indomethacin, 3-isobutyl-1-methyl-xanthine, and gentamicin. Adipogenic maintenance media contained only insulin and gentamicin. Control cells were incubated only in maintenance media. One week after the final round of induction, cells contained a large number of microscopic lipid vacuoles. The MSCs were fixed in 10% formalin for 45 minutes and washed with water and then 60% isopropyl alcohol. Oil red O was used to stain the adipogenic cells. To prepare this stain, 18 mL water was added to 27 mL of 3 mg/mL Oil red O in isopropyl alcohol. After ten minutes the solution was filtered and added to the fixed cells for five minutes followed by water wash. Cells were counterstained with hematoxylin for 2 minutes. Induction of chondrogenesis again used MSCs loaded and unloaded with SiNPs. Control media (Lonza) was supplemented with dexamethasone, ascorbate, gentamicin, sodium pyruvate, proline, and L-glutamine per the manufacturer's instructions. Induction media contained the same as well as 10 ng/mL transforming growth factor beta (TGF-β). Cell pellets containing 250,000 MSCs were created in 15 mL polypropylene tubes and induced for 3 weeks. Media was changed every 2-3 days. Pellets were fixed with 4% gluataraldehyde, 3% acetic acid, and stained with 1% Alcian blue and then suspended in 2% agarose that was embedded in paraffin after cooling. Sections 5 μm thick were sliced with a microtome and immobilized on positively charged slides.

### Cytokine Expression

Cells with and without SiNPs were plated at 20,000/cm² in a 12 well plate and cultured for two days. The media was then exchanged and allowed to stand for 24 hours. That media from positive and control cells was then removed along with media without cells that had been in the incubator for the same amount of time. Secretome analysis was performed with a bead-based assay (Luminex) by a commercial operator (Rules Based Medicine; Austin, Tex.)³⁸. Assays used 8 calibrations standards per protein and three controls. The antibodies for capture and detection were directed against all isotypes³⁹. Every protein was measured with a redundancy of 50 beads.

### Histology

Tissue sections were removed and immediately placed in 10% buffered formalin (Fisher) for two days and then transferred to 30% sucrose in PBS. Sections were then placed in optimal cutting temperature (OCT) media and froze for ten seconds in a bath of isopentane that was immersed in a bath of liquid nitrogen. Tissue sections (6 μm) were sliced and placed on charged slides. Immunofluoresence employed a goat anti-rabbit biotinylated primary antibody and Alexa 647-coated streptavidin. Sections were imaged with an automated histology slide reading tool (Nanozoomer; Hamamatsu).

### Microscopy

All transmission electron microscopy (TEM) and energy-dispersive x-ray spectroscopy (EDS) was performed with a Tecnai G2 X-Twin (FEI Co.) instrument operating at 200 kV. After loading with SiNPs, MSCs were washed three times with PBS, removed from the flask with tryspin, washed with media and saline using 5 min of 1000×g centrifugation to create the pellet and prepared for TEM as described previously⁴⁰. Briefly, cells were transferred to a 2:1:1 solution of 0.2 M sodium cacodylate buffer/10% glutaraldehyde/8% paraformaldehye (EMSdiasum). Samples were then stored at 4° C. before being stained en bloc with osmium tetroxide. After 2 h, samples were rinsed with deionized water and stained with uranyl acetate overnight. Samples were then dehydrated in progressively higher concentrations of ethanol in water: 50, 70, 95, and 100%. Samples were further dehydrated using propylene oxide and embedded in Embed 812 epoxy resin (EMSdiasum). Thin sections (500 nm) were cut using a Leica Ultracut S microtome and placed on a 200-mesh bare copper grid (Ted Pella). Fluorescence microscopy was performed with a Leica Confocal System and white light microscopy utilized an Axiovert 25 steromicroscope (Zeiss) fitted with an AxioCam CCD detector and MR Grab software.

### Ultrasound Imaging

Ultrasound imaging was performed with both a pre-clinical Vevo2100 system with M550 and M250 transducers (Visualsonics) or a clinical scanner (iU22; Philips) with L12-5 and L17-5 transducers. Optimal parameters for imaging SiNPs were obtained by empirical study of a 5 mg/mL SiNP sample including the frequency and power of delivered ultrasound, as well as the gain, range, and display of the acquired ultrasound image. The optimal settings at 40 or 16 MHz were 100% power, gain of 20 dB, and dynamic range of 50 dB. Video frame rate varied between 50 and 300 frames per second (fps). When contrast mode was used, it was in conjugation with the M550 transducer that uses linear contrast of the B-mode signal. At least five fields-of-view were collected for each sample.

To study the echogenicity of SiNPs ex vivo, we used agarose phantoms. A solution of 1% (wt./wt.) UltraPure Agarose (Invitrogen) in DDI water was prepared and boiled briefly and used with a 100×15 mm polystyrene Petri dish. To create the phantom, a 2-3 mm layer of agarose was poured and allowed to cool. Then, a 9-10 cm layer of agarose was poured on top of the already cooled layer. While the new layer was still in the liquid phase, the large end of a 1-200 μL disposable pipette tip (USA Scientific, Inc.) was placed into the liquid. After cooling, this pipette tip was removed to create an inclusion. This void was filled with the sample to be studied and mixed 1:1 with warm agarose. After sample cooling, a final layer of 1-2 mm of agarose was poured to seal the inclusion. The phantom was immersed in water prior to imaging.

### Animal Studies

Female nu/nu mice age 6-16 weeks were used in this study. All animal work was conducted in accordance with the Administrative Panel on Laboratory Animal Care at Stanford University. Prior to imaging, mice were anesthetized with 2% isofluorane at 2 liters/min and confirmed with tail pinch. Intracardiac delivery of MSCs was performed with a custom-made 27 gauge×1″ catheter, while subcutaneous or hepatic delivery utilized a 0.5 mL insulin syringe.

### MRI Characterization and Imaging

To measure the relaxivity of the SiNPs, various concentrations of SiNPs from 0.1 to 2.0 mg/mL were dissolved in DDI water. MRI imaging and T1/relaxivity measurements employed a M2 system from Aspect imaging that utilized a 1T permanent magnet. After auto shimming and scouting, 2 dimension T1 spin echo (SE) multislice images were acquired using vertical frequency directions, 100 mm fields of view, an echo time of 20 ms, repetition time of 500 ms, flip angle of 90°, a 512×512 matrix, 1 excitation, 20 μs of dwell time, and auto gain calibration. Imaging after injection of contrast or cells retained the prior gain settings. Eleven to 13 slices were acquired with no gap between slices, and 1 mm thick slices. The phi and theta angle were set to zero for axial slices with no echo asymmetry. The T1 was determined by measuring the exponential time constant in a plot of peak intensity versus delay between pulses. The reciprocal of T1 was plotted as a function of concentration. The slope of this line measures relaxivity of the SiNPs.

### Data Analysis

The limit of detection (LOD) was defined as signal detectable three standard deviations above the mean signal of the blank, and the limit of quantitation (LOQ) was defined as 10 standard deviations above the mean signal of the blank. Ultrasound image analysis was performed with Vevo and ImageJ software⁴¹. Images were saved as RGB TIFF files or .avi videos. Image quantitation was done with Image J (images) or with Vevo software (cine loops). Grayscale intensity was measured by region of interest (ROI) analysis. For the phantoms, signal was defined as the B-mode ultrasound contrast generated by the SiNP inclusion unless otherwise specified. Background was defined as the B-mode ultrasound contrast generated by the agarose gel surrounding the inclusion. Noise was defined as the standard deviation in the signal in areas without contrast. Signal to noise was calculated by dividing signal by noise.

In animals, contrast mode in the ROI was used to determine increase in signal via subtraction of the pre-injection image from the post-injection image and indicated areas of increased B-mode signal. Those pixels that had greater signal than the pre-injection pixels were assigned to a green lookup table and then overlaid with the original image. Contrast mode videos used the average of at least 100 frames prior to injection of MSCs as the baseline above which subsequent frames were compared. Subsequent pixels 20% above this average were coded green by the Vevo software. Maximum intensity projections sequentially added additional pixels throughout the cine loop. For LOD determinations, we used the mean intensity of the ROI for each animal subject post injection.

Data analysis of MRI images utilized DICOM files and ImageJ. Distance measurements utilized MIcroDICOM software (MicroDicom). For the LOD calculations in MRI, ROIs were drawn around the injected cell bolus for those frames containing cells. Because the size of the implant was different between animals, we used the integrated density of the ROI rather than the mean. The integrated density values for those frames were summed and defined as signal. Because the imaging software scales each imaging session with a unique scaling factor and because of differences in receiver gain between studies, we normalized the value of signal by the mean intensity of latissimus dorsi muscle in the same slice as the cell bolus. That value was plotted as a function of injected cell number. For time course studies, we used the mean intensity of one DICOM slice normalized by muscle intensity again using ImageJ.

### Results

### SiNPs Characterization

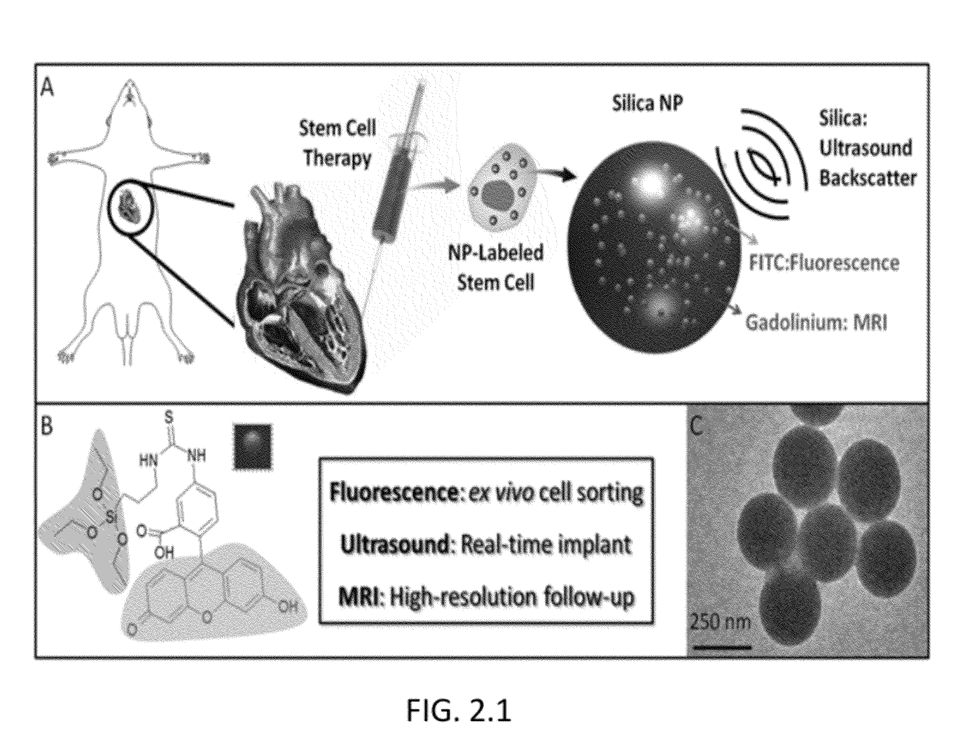

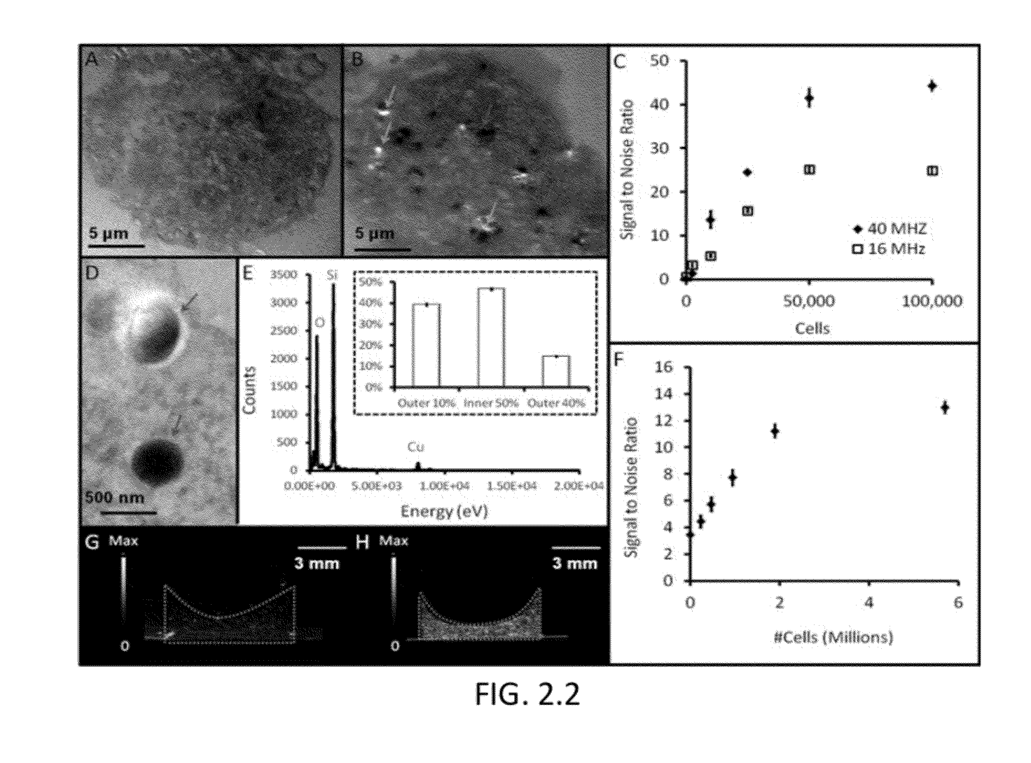

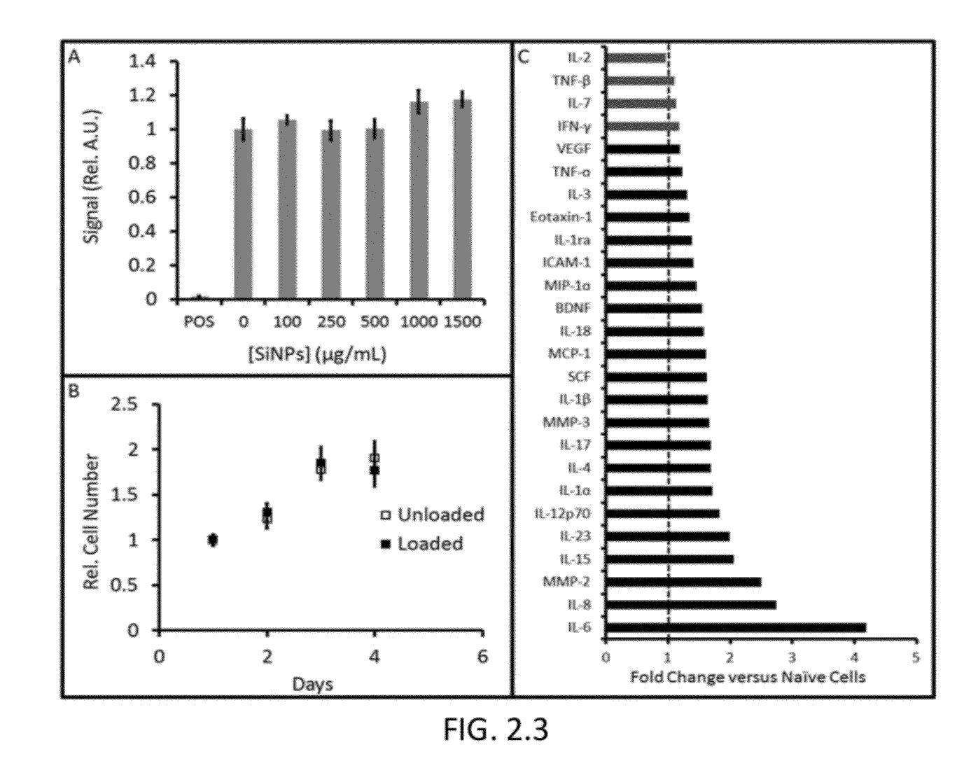

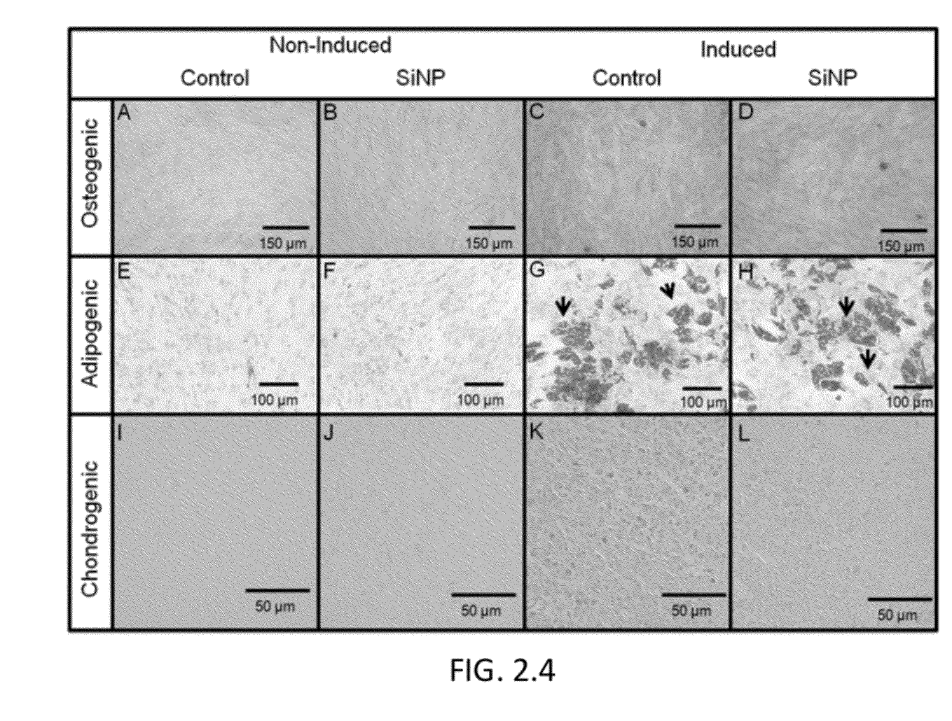

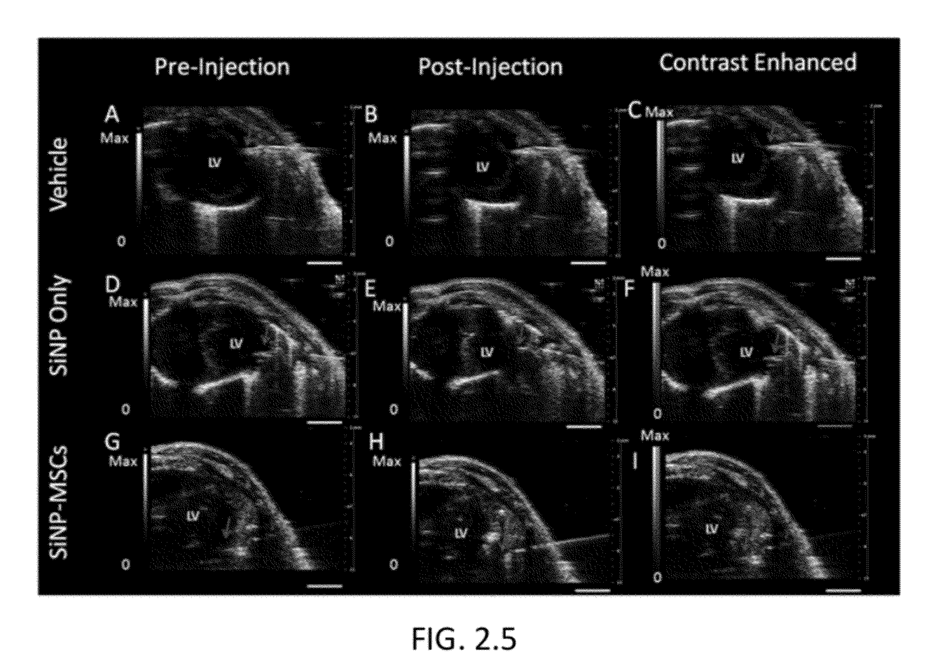

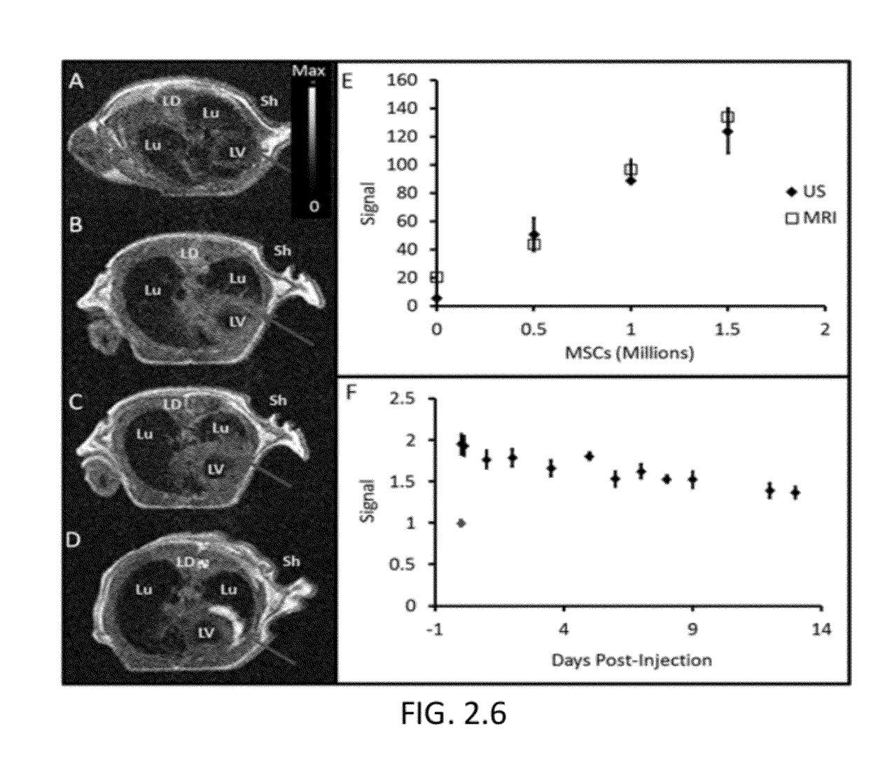

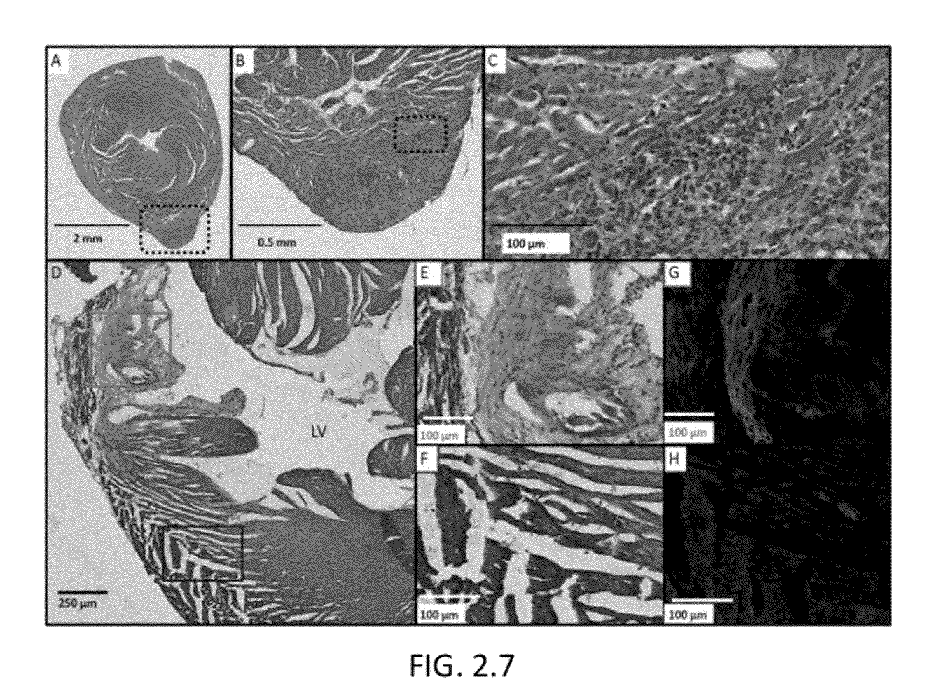

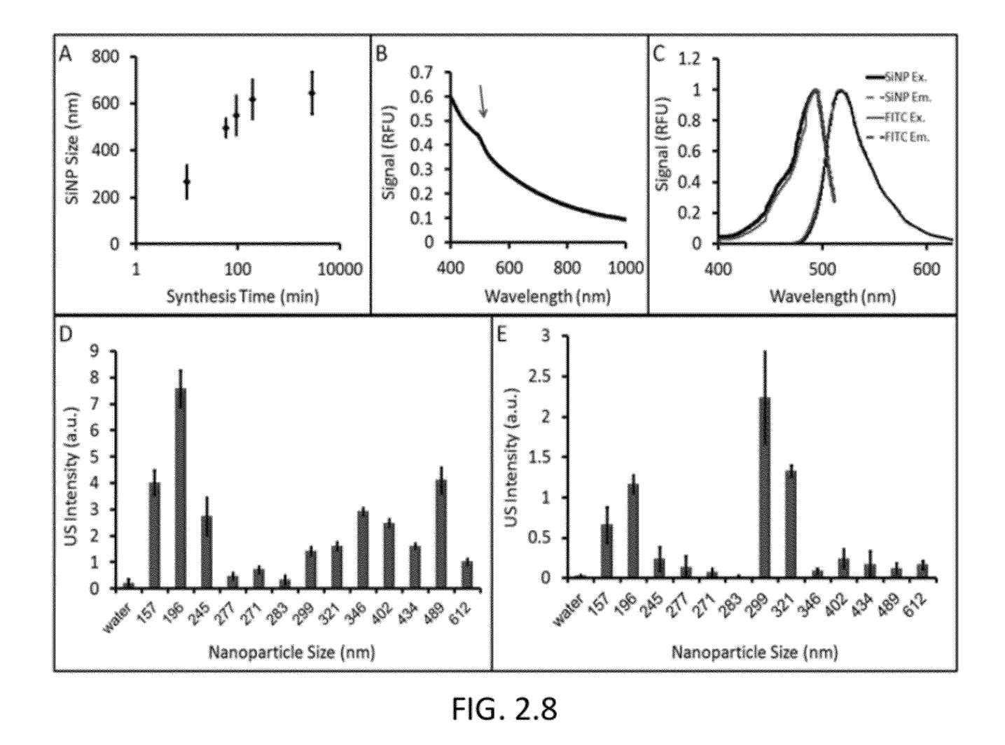

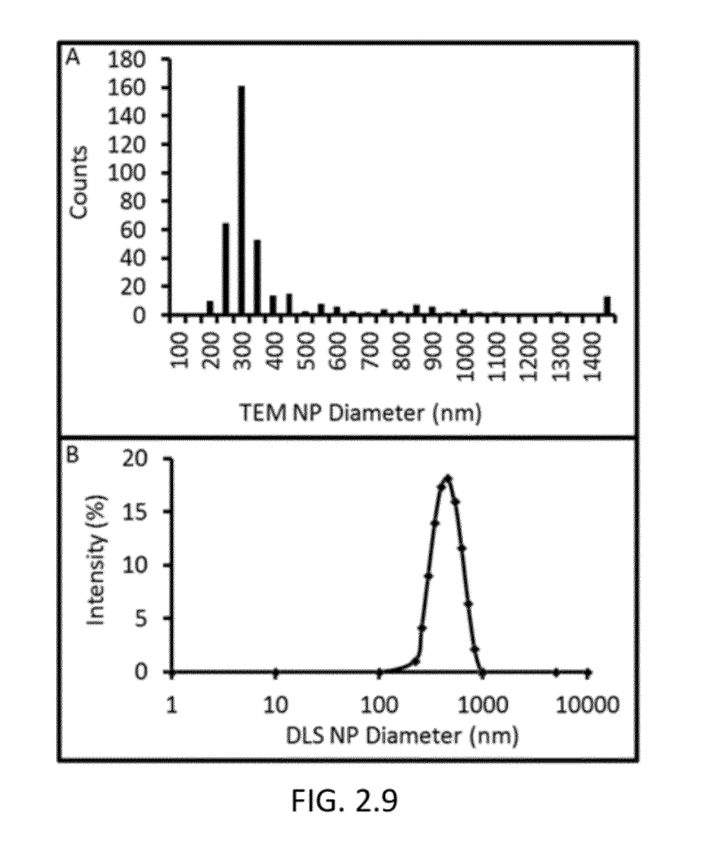

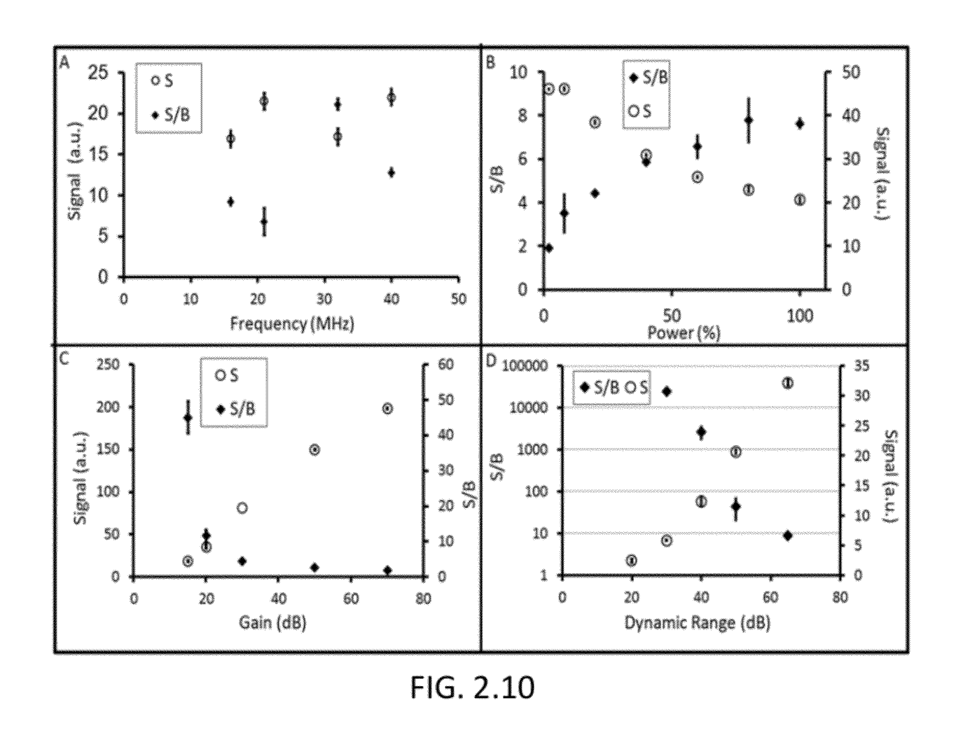

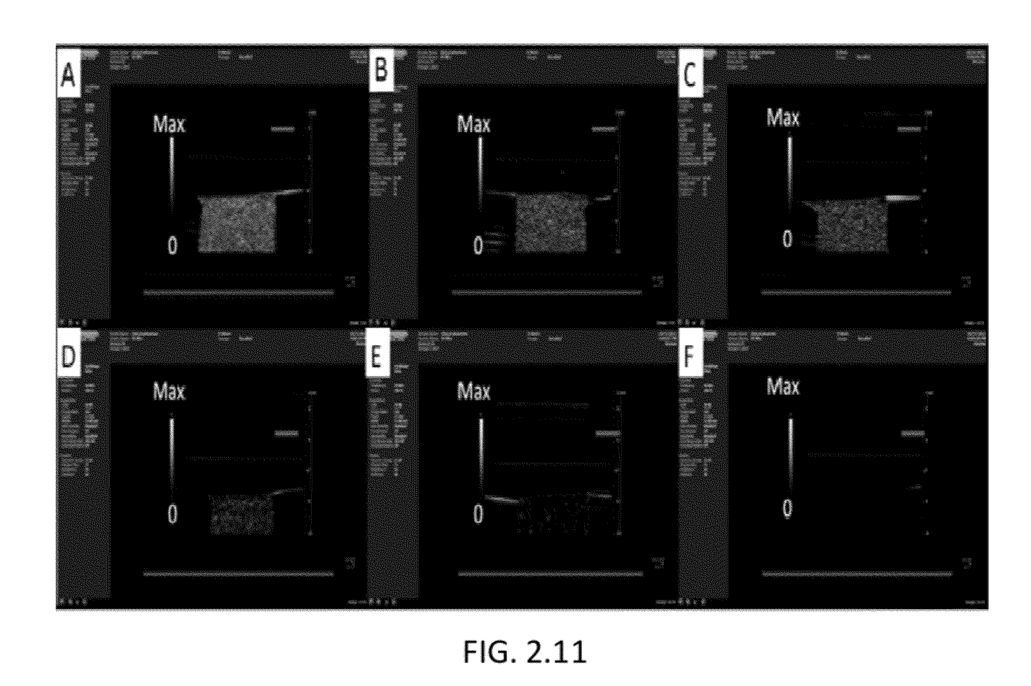

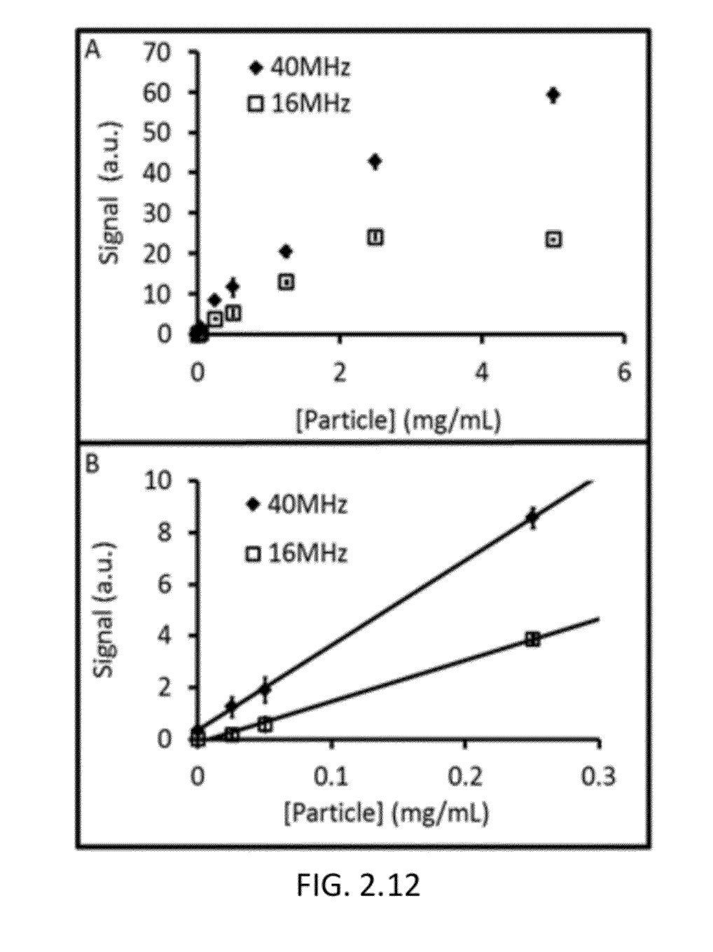

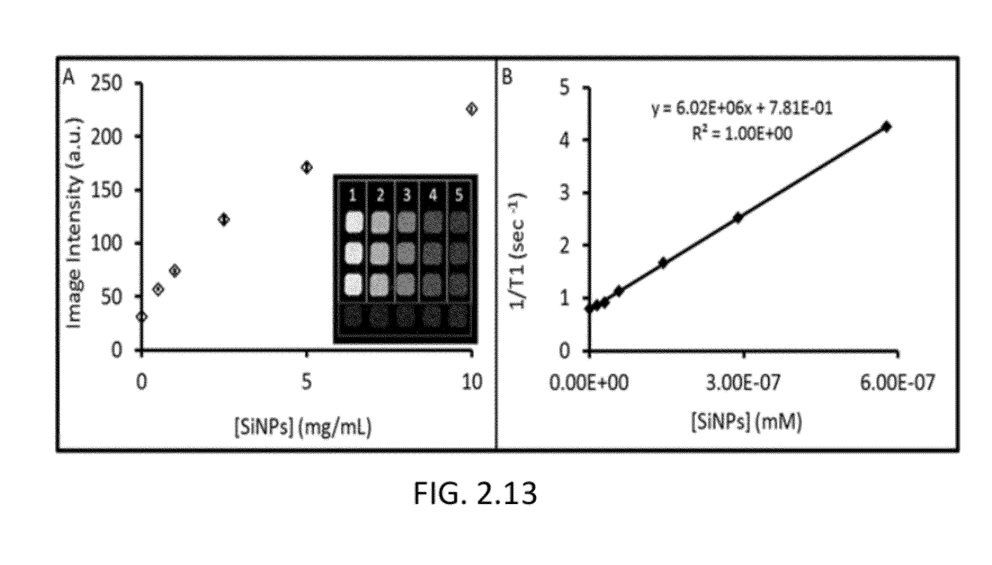

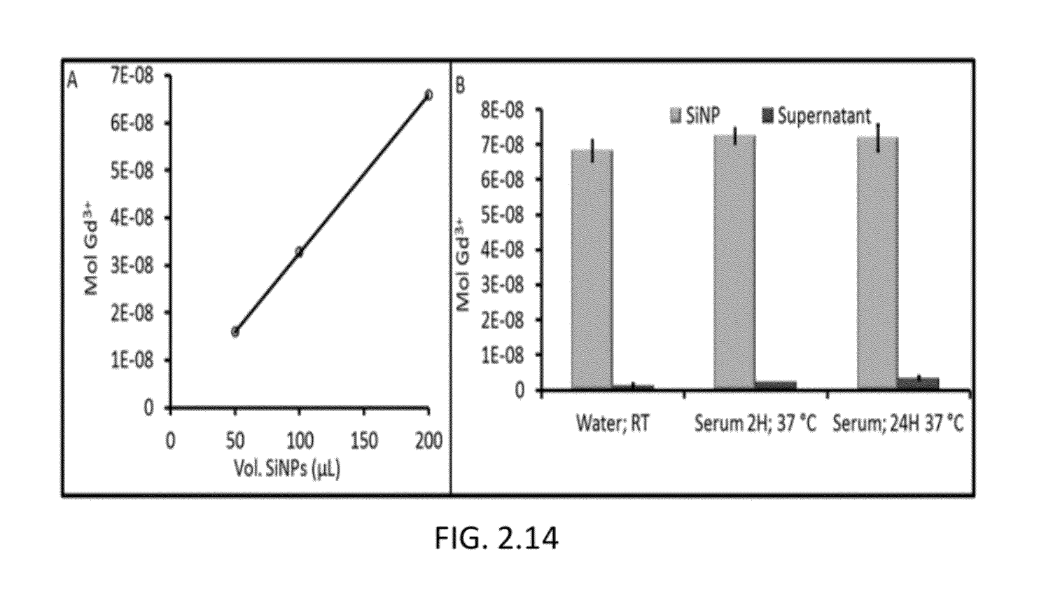

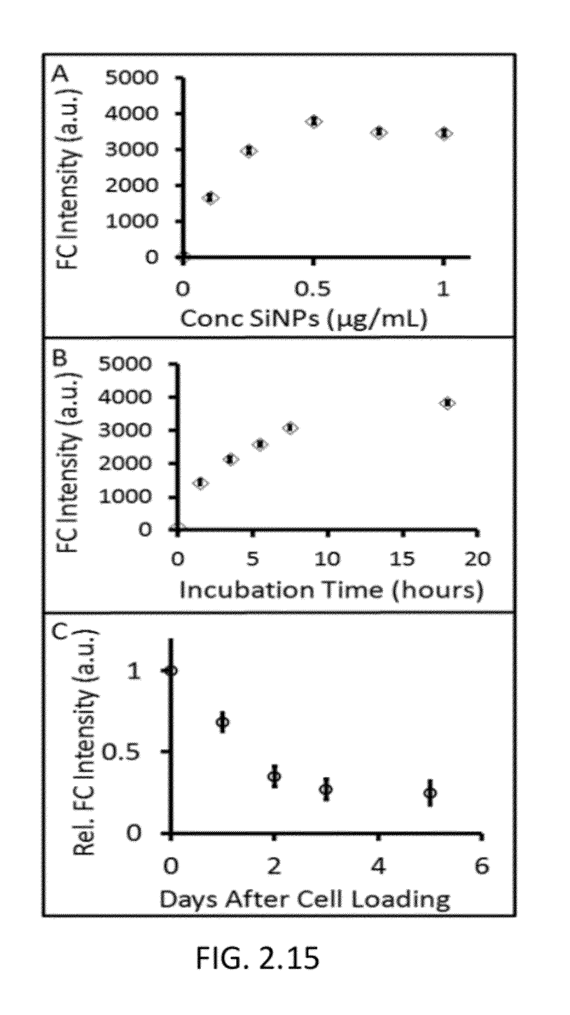

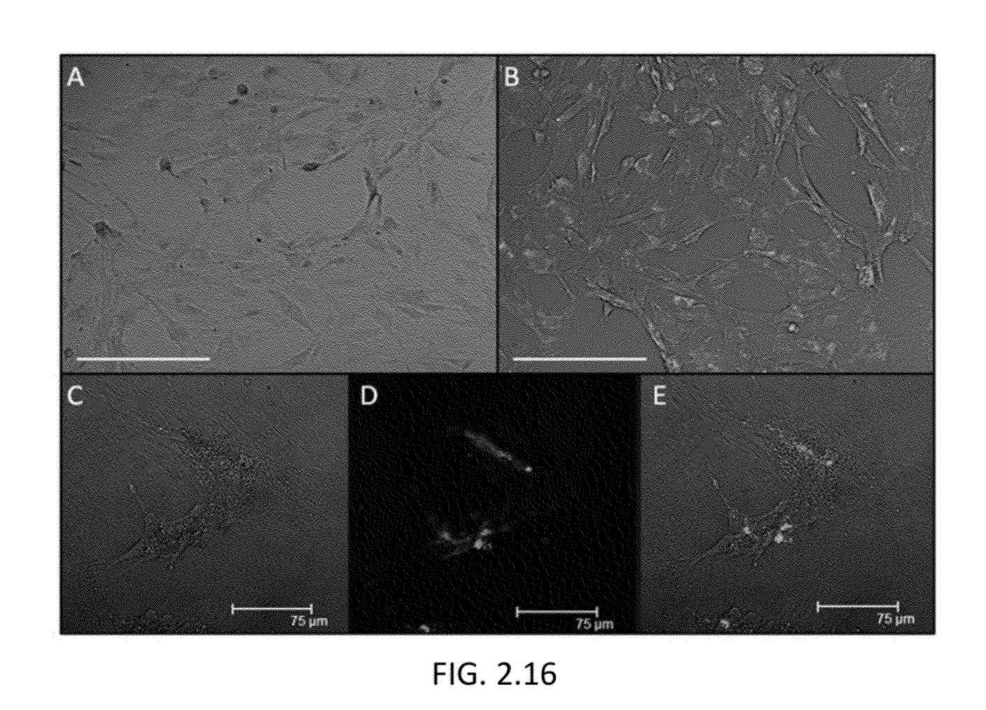

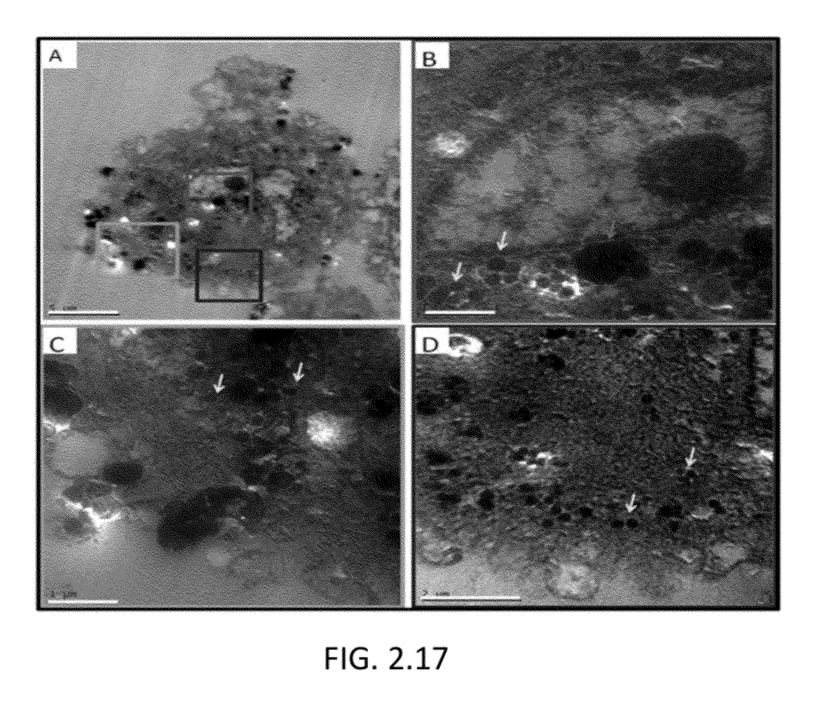

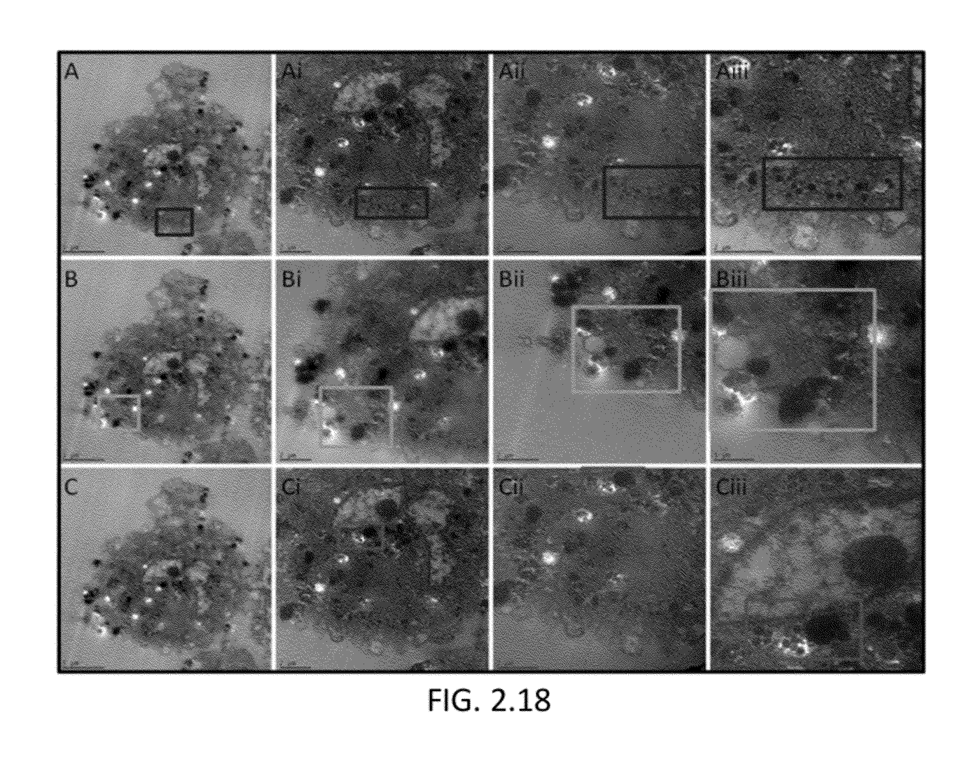

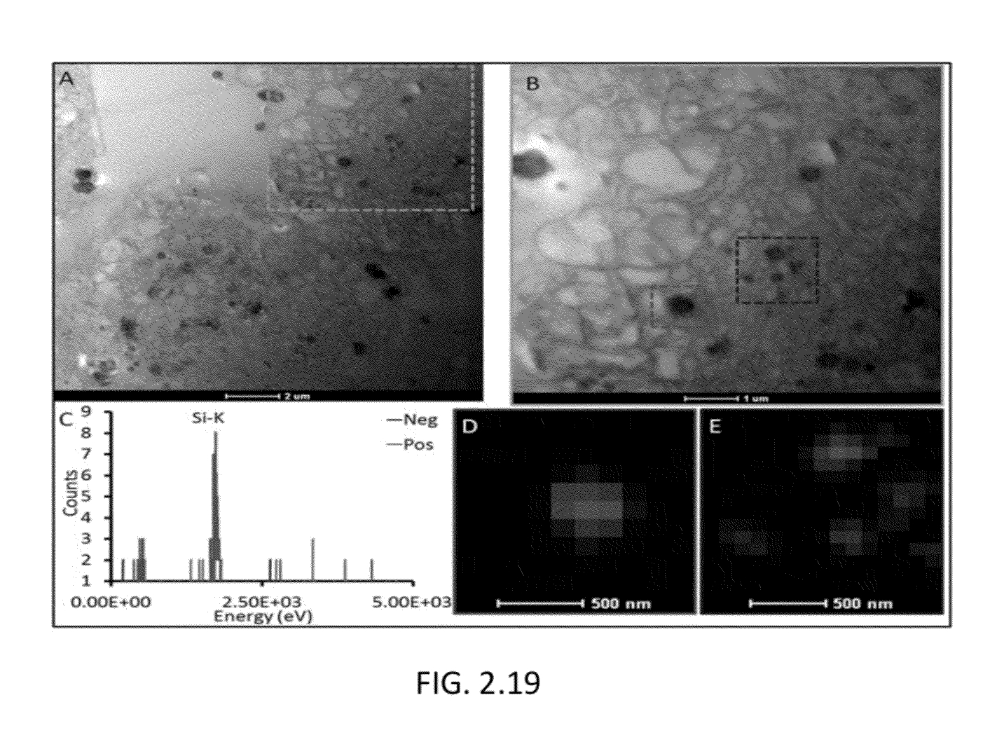

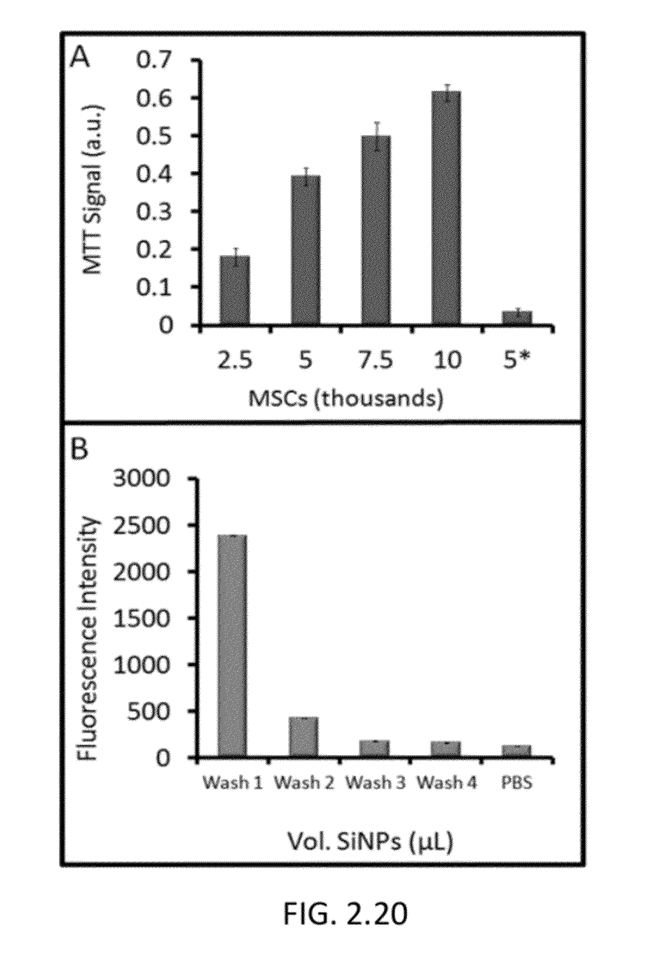

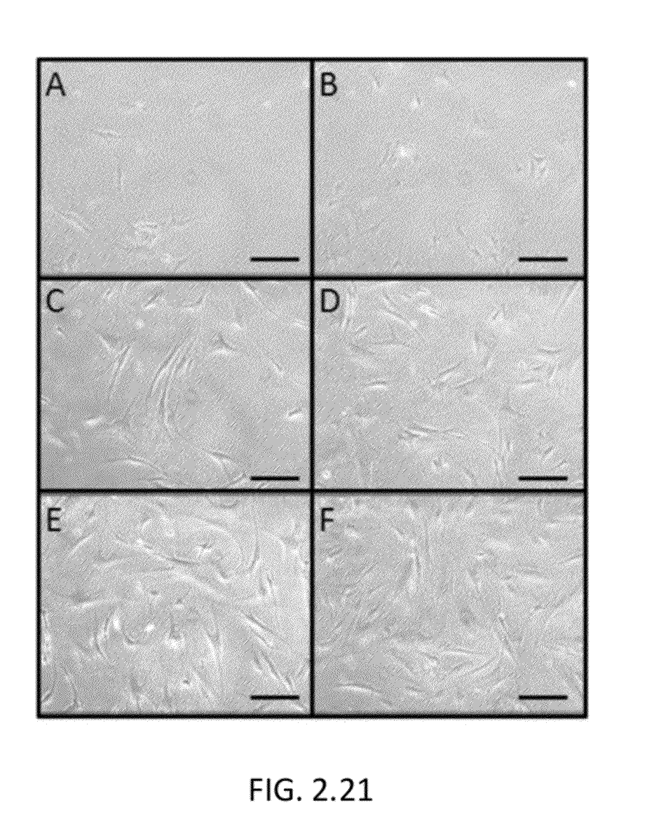

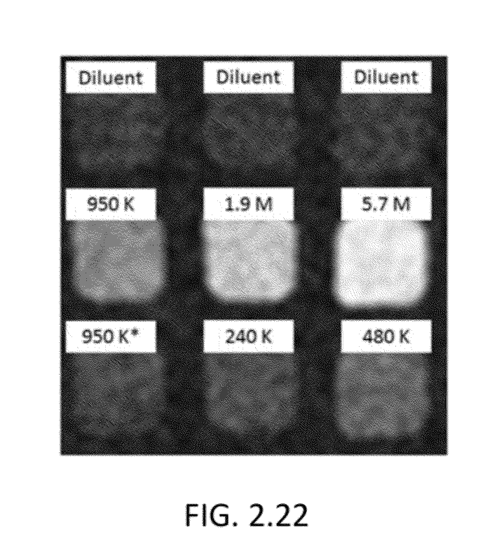

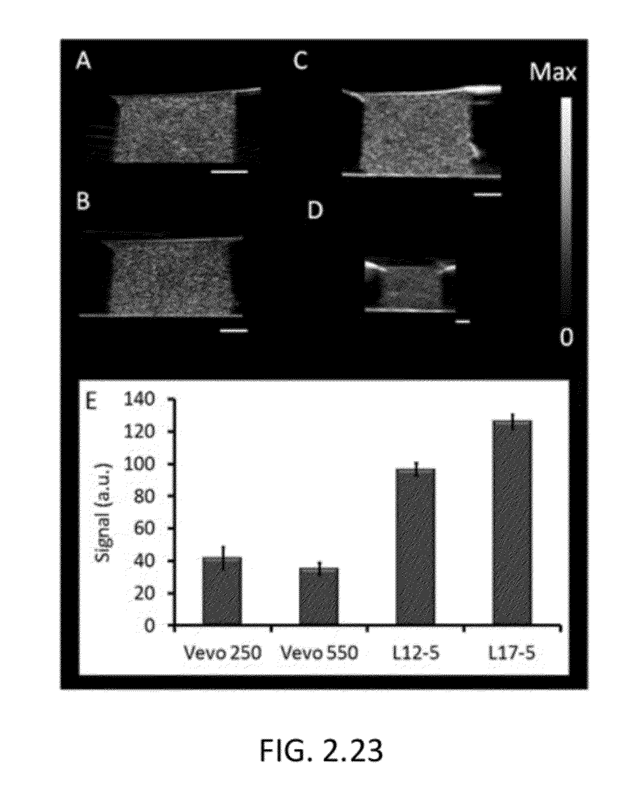

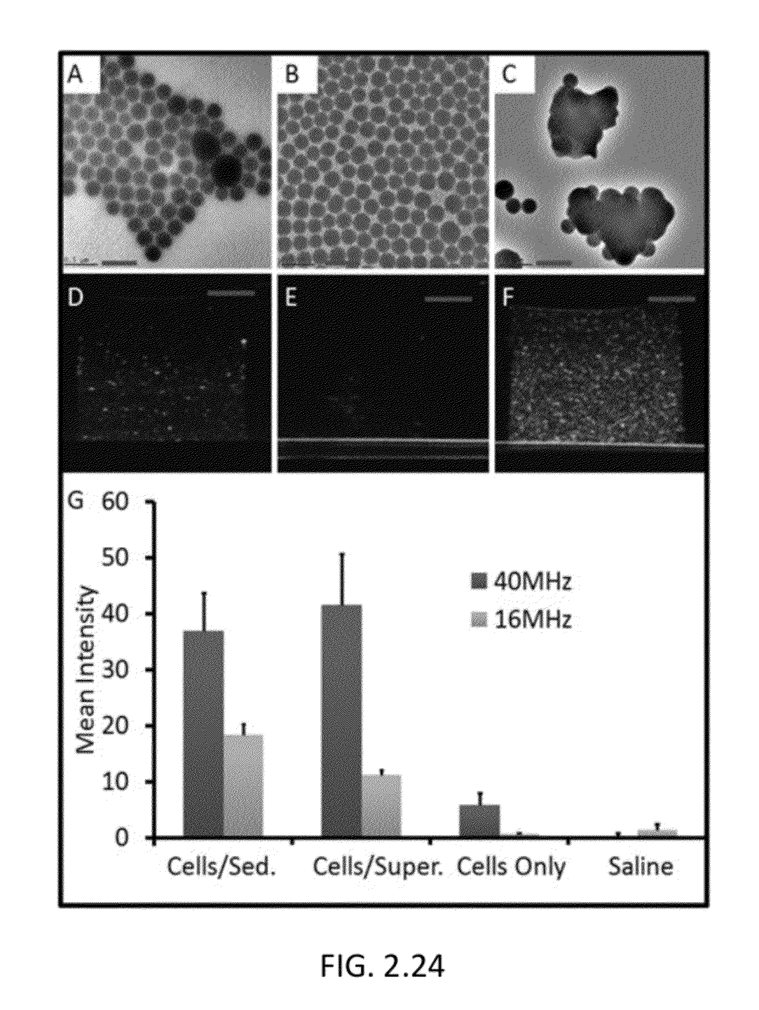

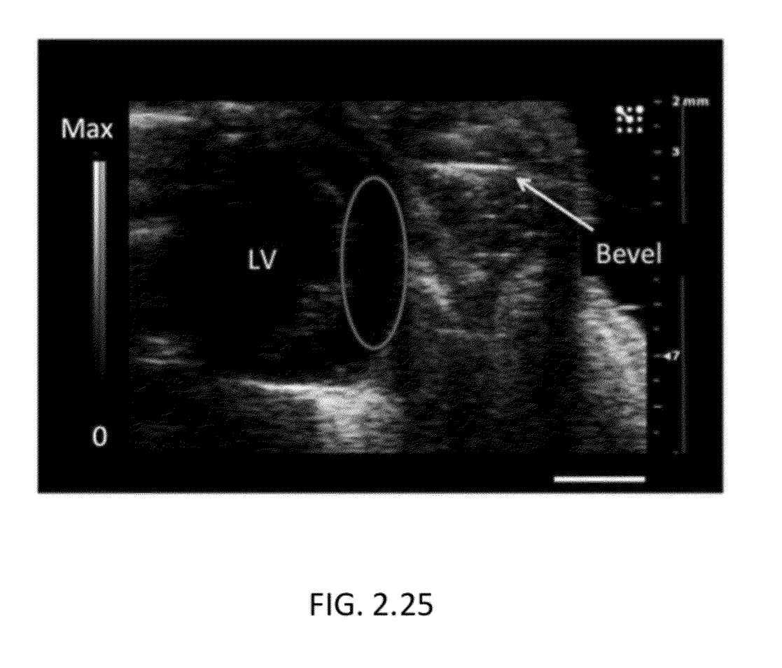

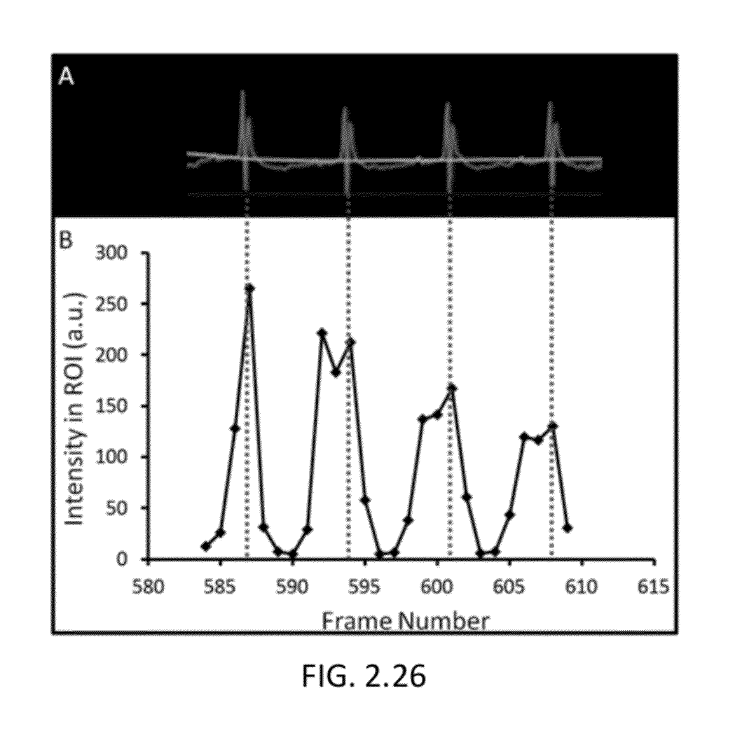

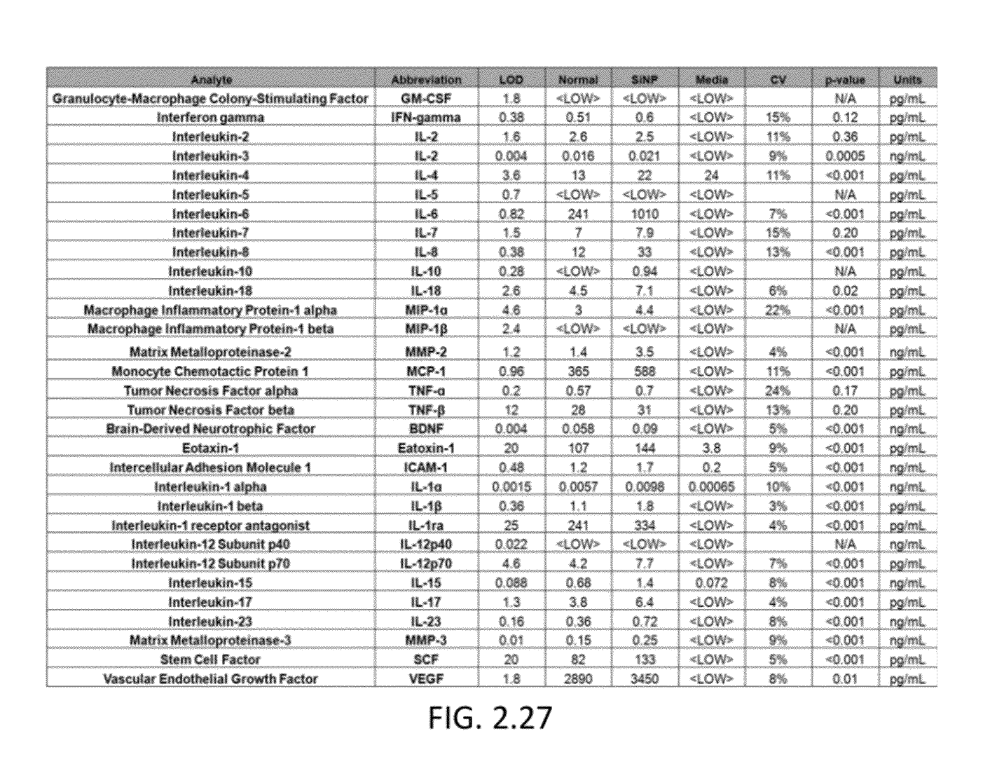

One initial goal was to measure the physical and signaling performance characteristics of the SiNPs (FIG. 2.1). We monitored the growth of the nanoparticles using DLS and observed rapid growth in the first three hours after addition of TEOS to the reaction mixture (FIG. 2.8). The typical yield was 250 mg±30 mg per batch with no changes to excitation and emission features of FITC (FIG. 2.8C). We characterized the physical size of the product with DLS and TEM. Size was determined by TEM by imaging 45 different fields-of-view (FOV), each with more than 20 SiNPs per FOV. Size analysis of these images is presented in FIG. 2.9A and indicates a mode size of 300 nm and a mean size of 403.8±342.8 nm. The size (hydrodynamic radius) was also determined by DLS and the Z-average value was 600 nm with a polydispersity index of 0.144 (FIG. 2.9B). The batch-to-batch variation in size of the product for these four different lots was 25% by TEM. The zeta potential of these materials was −6.5±6.2 mV in water and −29.0±−16.0 mV in 50 mM saline.

The capacity of the SiNPs to generate contrast in the US, fluorescence, and MRI (T1-reducing) modes was studied. The relative standard deviation (RSD) of fluorescence between batches at 0.5 mg/mL was 21.3%. As US was the focus of this study, we serially diluted the SiNPs between 10 and 0 mg/mL in an agar phantom and analyzed the specimens at 16 and 40 MHz with optimized gain, frequency, power, and dynamic range (FIG. 2.10). Representative fields-of-view for each concentration are shown in FIG. 2.11 and the calibration curves at the two frequencies are shown in FIG. 2.12. The limit of detection (LOD) and limit of quantitation (LOQ) at 40 MHz is 11.5 μg/mL and 100.5 μg/mL, respectively. At 16 MHz, LOD and LOQ are 18.5 μg/mL and 22.5 μg/mL, respectively. The RSD in ultrasound intensity as measured at replicate samples (n=4) of 1.25 mg/mL is 3.0% and 2.4% at 40 and 16 MHz, respectively (intra-batch relative standard deviation (RSD)). The variation (RSD) between batches was 11.1% at 40 MHz and 5.5% at 16 MHz. The T1 shortening capacity of the SiNPs was evaluated with tissue phantoms and spin echo imaging (FIG. 2.13). The LOD was 29.0 μg/mL and concentrations above 10 mg/mL showed a decreased signal due to water exclusion. Finally, the relaxivity of the SiNPs was measured via decreasing concentrations of material and found to be 6.02×10⁶/(mM s) at 1.0 T with batch-to-batch signal variation of 1.04% (FIG. 2.13B). Relaxivity per Gd³⁺ is 4.1/(mM s).

The amount of Gd³⁺ per SiNP was determined via ICP analysis. A dose response curve for triplicate measurements of three different amounts of SiNPs was linear (R²>0.99; FIG. 2.14) with intra-measurement RSD<3.5%. An average of 1.47×10⁶±3.06×10⁴ Gd³⁺ ions were determined per SiNP for the nine measurements. Free Gd³⁺ in the solution after dialyzing the SiNPs was less than background signal of diluents (0.037 ppm). The serum incubation study indicated 4.75% and 3.22% of the Gd³⁺ became dissociated from the SiNP at 24 and 2 hours of incubation at 37° C., respectively. Incubation in water caused 1.80% dissociation. The RSD of these measurements were between 2.99% and 5.06% (FIG. 2.14B).

### Cell Labeling

The capacity to label MSCs with SiNPs was optimized in cell culture. MSCs were incubated with various concentrations of SiNPs between 0 and 1.5 mg/mL of SiNPs and incubated for 7.5 hours. Concurrently, MSCs were incubated at 0.5 mg/mL for various amounts of time from two hours to overnight. Cells were analyzed by flow cytometry (FC) and the results (FIG. 2.15) suggest that the optimal loading is 0.5 mg/mL. Although a slight benefit is seen to overnight incubation, for convenience, 6 hour incubation times were used for the remainder of the experiments. Thus, 0.5 mg/mL of material for 6 hours was used for all subsequent labeling protocols. To be sure that all SiNPs were contained within MSCs, we analyzed the fluorescent signal of the successive PBS washes (after SiNP loading, but before trypsin treatment) as shown in FIG. 2.20.B. After three washes, no fluorescence is observed suggesting that all SiNPs are contained within adherent cells. To measure the temporal stability of loading, 25,000 loaded MSCs were re-plated in each well of a 12 well plate. The FC signal from the FITC reporter was measured daily and the relative intensity is plotted in FIG. 2.15C and suggests that by 48 hours the cell intensity is reduced by 50%, which corresponds to the doubling time of 2 days. At this rate, the cells would still be detectable at generation five.

Cell loading was confirmed with microscopy. Before MSCs were removed from the plate, they were analyzed with light and fluorescence microscopy. FIG. 2.16 shows FITC signal distributed throughout the cells. Confocal microscopy was used to better understand the 3-dimensional distribution of SiNPs throughout the cells and suggest that SiNPs were located throughout the MSC interior and are not relegated to cell exterior (FIG. 2.16C-E). As a secondary confirmation of labeling, TEM shows nanoparticles throughout a 500 nm section of a MSC in FIG. 2.2 as well as a control MSC not labeled with SiNPs. The TEM images of 40 unique 500 nm sections indicated that 16.6±3.5 SiNP clusters were located per slice with a mode SiNP size of 1100 nm and a mean size of 1400±500 nm. FIG. 2.17 presents additional TEM images that illustrate the wide variety of SiNP sizes that are present inside the MSCs. FIG. 2.18 offers increasing magnification to illustrate SiNPs for an individual MSC and FIG. 2.19 presents scanning TEM (STEM) imaging with maps of the Si K-edge signal from the SiNPs inside one MSC. Extrapolation to the total cell volume gives approximately 850 SiNP clusters per cell. Analysis by ICP indicated 6540±620, 300 nm SiNPs per cell. The diameter of MSCs was used to calculate a volume of 2.3×10⁵ μm³ and a cellular concentration of SiNPs of 1.0 mg/mL. TEM determined the location of the SiNPs. The location was assigned to either the center 50% of the cell area, outer 40% of the cell area, or periphery of the cell. In 83% of the analyzed cells, a majority of the SiNPs were present inside the cell, rather than the periphery.

### Impact of SiNPs on MSC

We next performed a series of experiments to measure the impact of SiNPs on MSC metabolism, viability, proliferation, cytokine expression, and pluripotency. In the first experiment, 20,000 MSCs were incubated with 0 to 1.5 mg/mL of SiNPs for 6 hours and their metabolic activity was then probed with the MTT assay. (The utility of MTT with MSCs was first confirmed via simple cell counting; FIG. 2.20A). There was no statistically significant (p>0.05) difference between the cells with no SiNPs and those up to 1.5 mg/mL (FIG. 2.3A). A positive control that utilized cetyl trimethylammonium bromide (CTAB) as a cytotoxic agent validated the assay. Readout was A570 nm-A630 nm.

Second, MSCs both loaded and unloaded with SiNPs were plated at 5,000 cells/well in each well of a 96 well plate. 24 hours later the MTT assay was performed and on each subsequent day for four days. There was no difference in the growth rate between the two cell populations (FIG. 2.3B). After four days, further cell growth is mitigated by crowding in the wells. FIG. 2.21 white light microscopy images of cells at 1, 3, and 7 days of growth. No difference in size, shape or morphology was noted at the normal 0.5 mg/mL loading level. At 1.5 mg/mL, the cells lose some spindle-like character adopting a more spherical architecture.

Third, to determine whether normal metabolic processes within the MSCs caused leaching of Gd³⁺ ions from the SiNPs, MSCs loaded with contrast agent were re-plated and allowed to grow for 24 hours. The media from the wells (n=4) was then measured for Gd³⁺ content and found to be 7.7±5.0 ng/L. Media control in the absence of MSCs (n=3) was 7.4±1.7 ng/L. The p-value between the two sample sets was 0.93 suggesting there was no statistically significant difference in Gd³⁺ content between media and the media from metabolically active SiNP-loaded MSCs. All Gd³⁺ was contained within the SiNPs and isolated inside the MSCs.

Fourth, we analyzed the secretome of SiNP-loaded and control MSCs. Of the 31 analyzed proteins, 26 had levels in cell culture media that were measureable by the bead-based Luminex assay; FIG. 26, Table S1³⁸. We divided the cytokine concentrations from the labeled MSC secretome to unlabeled MSC secretome to construct FIG. 2.3C. All proteins were within 1-fold of baseline except IL-6, IL-8, and MMP-2.

Finally, we used differentiation reagents to determine whether the SiNPs impacted the pluripotency of the MSC⁴². This work sought to answer two questions: 1) Can SiNP-loaded MSCs still differentiate²⁸? and 2) Does the presence of SiNPs induce any unintended differentiation? We were especially concerned that the SiNPs might unintentionally transform MSCs into osteogenic cells as silicon-based structures have previously been show to induce osteogenic differentiation⁴³. Fortunately, SiNP-loaded cells were still easily transformed into ostegenic and adipogenic cell lines (FIG. 2.4). There was 4.9-fold more osteogenic signal (as determined by A402) in the induced (FIG. 2.4D) cells than non-induced cells (FIG. 2.4B). Adipogenic induction produced many lipid-containing vacuoles in both the control (FIG. 2.4G) and SiNP-containing cells (FIG. 2.4H). Cell labeling has been reported to inhibit chondrogenesis in MSCs^{44,45}. We noted an 18.9% reduction in staining intensity of the SiNP-labeled MSCs relative to unloaded controls. Nevertheless, the faint but consistent Alcian Blue staining suggests this pathway does remain intact.

### Signaling Capacity of SiNP Transfected MSCs

The agarose phantom experiment was repeated with SiNP-loaded MSCs and unlabeled MSCs. When 50,000 of each type were measured, the labeled cells had 3.0 and 2.4-fold higher B-mode signal at 40 and 16 MHz, respectively (FIG. 2.2G, H). This difference was significant at p<0.001. When decreasing numbers of cells were added to the phantom and imaged as described above, the LOD for 16 MHz was 1,430 MSCs and 635 cells at 40 MHz (FIG. 2.2C). Cells were also imaged with T1 SE MRI imaging (FIG. 2.2F and 2.21). Decreasing amounts of cells from 5.7×10⁶ to 2.4×10⁵ were imaged in a 384 well plate. The LOD above the background of the saline diluents was 338,000. One experiment (FIG. 2.21) compared the signal intensity of 950,000 loaded and unloaded cells. The loaded cells were twice as intense as the native cells.

To confirm that the SiNPs were consistent with clinical ultrasound scanners, we scanned contrast agent alone, 40,000 SiNP-loaded MSCs, and an agarose blank using the Vevo pre-clinical scanner and a Phillips iU22 clinical scanner (FIG. 2.22). The LOD of contrast agent with the clinical scanner was 17 μg/mL SiNPs and 40,000 SiNP-labeled MSCs were easily visualized with the clinical system (FIG. 2.22E). The LOD at clinical frequencies is 14,000 MSCs. The signal-to-background ratio of these cells versus the agarose/saline vehicle was 9.2 and 10.9 for the 40 and 16 MHz preclinical transducers, respectively. On the Philips clinical system, the values were 8.3 and 6.7 for the L12-5 and L17-5 transducers, respectively.

The study presented in FIG. 2.23 suggests that size plays an important role in the generation of contrast. The SiNPs were allowed to settle overnight and separate into the larger, irregular sedimented particles and the smaller supernatant. The sediment yields higher US contrast in phantoms (36-fold higher than that of the smaller supernatant). However, when these two fractions were used to label cells, there was no statistical difference between the two populations (p=0.38) with a 7-fold increase in signal above unlabeled cells.

### Utilization of SiNP in Cardiac Applications

A final study investigated the capacity to image labeled SiNPs after intra-cardiac injection in mice. All echocardiograms are in short-axis mode and injections employed 100 μL 50% PBS/50% matrigel into the wall of the LV. Images were collected before and after injection. To interrogate the contribution of traumatic process of cell injection to the observed US signal, we did a sham injection (matrigel only) and observed no increase in B-mode signal (FIG. 2.5C). To confirm that capacity of the SiNPs to generate contrast in the cardiac space, we next injected SiNPs only (6 mg in 100 μL 50% PBS/50% matrigel) into the LV and saw increased B-mode signal (380%) at the site of injection (FIG. 2.5F). Finally, 1,000,000 SiNP-loaded MSCs were injected into the LV and increased B-mode signal (640%) was seen at the injection site (FIG. 2.5I). Cells are clearly seen entering at the 0:11.

Movement was reduced to create contrast enhanced video of the injection process. Movies were performed away from the movement-rich thick ventral wall near the apex of the heart. These videos also use both respiratory gating to disregard frames near peak animal movement and also gate via the ECG response. Frames were collected 50-100 ms after the QRS complex, depending on animal heart rate, which was maintained near 400 beats per minute (bpm) via fine tuning the anesthesia. One movie is the B-mode video of a typical injection with an ROI drawn to highlight the treated area with injection near 0:12. Another offers a contrast enhanced view using the first 115 frames as the reference frames. Pixels above this background are coded green. Another is the same sequence, but processed as a maximum intensity projection (MIP). To determine how much of the MIP signal was due to motion in the chest cavity, the injection was in two stages. In another video, cells are initially injected near the 0:15 point. No cells are injected from 0:18 to 0:26 and no increase in contrast is seen. Injection of MSCs is resumed after 0:27 and along with a corresponding increase in MIP signal. Importantly, the use of US allowed immediate identification of mis-injected cells (FIG. 2.24).

We next investigated the capacity of loaded MSCs to be imaged via T1 SE imaging after implantation via US-guided injection. Mice were injected with either 1.5×10⁶ SiNP-loaded MSCs in 100 μL 50% PBS/matrigel or the PBS/matrigel mixture only. FIG. 2.6 shows axial sections of the murine chest cavity before and after injection of both types of contrast. The labeled cells are clearly visible in FIG. 2.6D and the average increase in T1-weighted signal for the study was 250±18%. To confirm that MSCs were embedded in the wall of the LV, we measured the wall thickness before (2.25±0.1 mm) and after (2.49±0.4 mm) injection via T1 SE images and found that there was no significant (p=0.4) difference between the two. We also observed movement of a MSC bolus (in US) relative to the LV (as determined by electrocardiogram) and observed precise integration between the two (FIG. 2.25).

The capacity of the SiNPs to quantitate MSCs in vivo was determined by injecting increasing amounts of MSCs into the cardiac space of nu/nu mice (n=3 animals at each point). The animals were imaged with both US and MRI and the intra-cardiac LOD was 250,000 MSCs via MRI and 70,000 via US (FIG. 2.6E.) The subjects injected with 1.0 million cells were longitudinally monitored via MRI and the signal was elevated above baseline for 13 days (p<0.05).

We confirm that the signal seen by US and MRI indeed correlates to MSCs via histology. In one study, mouse hearts were excised 24 hours post injection, preserved, and imaged with H&E staining. FIGS. 2.7A-2.7C show increasing magnification of a treated area of the left ventricle. The darker purple area protruding from the ventricle wall indicates MSCs. In another set of mice (n=3), the heart was removed 10 days after implant and imaged with H&E as well as fluorescence imaging in both treated (FIG. 2.7D red box) and untreated (FIG. 2.7D blue box) areas of the heart. While both treated and untreated areas are positive for troponins (red; FIG. 2.7G and 2.7H), only the treated area presents green fluorescence indicative of the MSCs.

### Discussion

We used SiNPs to label MSCs and follow their fate in living subjects. The fluorescent mode was helpful during cell culture and facilitated microscopy and FC. The ultrasound mode was used during the implantation step and offered real-time guidance of both the needle and the cell bolus. Finally, the MRI mode offered high-resolution studies and to confirm the location of MSCs as well as for long-term follow-up studies.

The synthesis of the SiNPs was simple, completed within 24 hours, and utilized easily obtained starting materials with Gd³⁺ ions electrostatically immobilized by the negative surface charge of silica and oxygen lone pairs. The size was characterized by both DLS and TEM, with important differences observed between the two techniques (FIG. 2.9). The value determined by DLS (−600 nm) is larger than TEM (300 nm) as this technique measured hydrodynamic radius as opposed to TEM studies under high vacuum. DLS is also biased by larger particles to a much higher degree and is influenced by the 15% of the SiNPs above 500 nm on the histogram in FIG. 2.9A⁴⁶. Still, a relatively monodisperse population was observed with a PDI of 0.144. Although future work will seek a more uniform size distribution, this initial range was suitable for imaging in all three modalities.

We confirmed the location of the SiNPs in the MSCs through microscopy. Loading MSCs with SiNPs did not need transfection reagents and the internalization of silica structures has been described by many cell lines^{47,48} including HeLa⁴⁹ and MSCs⁵⁰. Previously, uptake of mesoporous SiNPs by MSCs was inhibited by phenylarsine oxide and cytochalasin D suggesting a clathrin- and an actin-dependent endocytosis⁵⁰. The mechanism has been shown to be folic acid or caveolae-mediated in other cell lines⁴⁹. MSCs containing SiNPs showed no changes to cell morphology, metabolism, growth rate, or pluripotency (FIGS. 2.3, 2.4, and 2.21). There was also no measureable metabolic toxicity even at concentrations 3-fold the standard loading level of 0.5 mg/mL (FIG. 2.3A).

Microbubbles are highly echogenic contrast agents, but offer poor ultrasound signaling capabilities in cell tracking applications. They have low internalization potential due to their large size. Those microbubbles that are internalized have a short half-life (5-10 minutes in vivo)⁵¹. The SiNPs are present both on the cell exterior and interior with the majority (60%) of material inside the cell. SiNPs are stable for many weeks. Contrast agent loading on the cell interior is critical for SCT as imaging agents not directly attached to the cells could be incorrectly interpreted as cells. Over 60% of the approximately 6,000 SiNPs loaded per cell were shown to be on the cell interior. Importantly, this cell loading level provides an internal cellular concentration of 1 mg/mL of SiNPs, which is well above the LOD determined for SiNPs in FIG. 2.12, but still shown to be a non-toxic dose (FIG. 2.3A).

The SiNPs are highly echogenic and are compatible with a wide range of US frequencies including those used clinically. Higher frequencies (40 MHz) are useful for high resolution imaging (sub-micron), while lower frequencies (<20 MHz) offer a better depth of penetration⁵². The intra-cardiac LOD was 250,000 MSCs via MRI and 70,000 via US. The temporal resolution achieved with this technique is 3.3 ms, which is 10,000 times lower than the next nearest real time, deep tissue, cell tracking technique, PET/SPECT⁵³. Although low numbers of cells could be measured, such limits of detection are rarely clinically applicable as human cardiac SCT typically involves millions of cells^{54,55}. An important feature of cell tracking methods is quantitation. The relationship between number of cells and the US and MRI signal was very linear (R²>0.98 for both methods), which suggests the method is quantitative in this regime of cells. Cell counting below 2×10⁶ MSCs was also linear in vitro (FIG. 2.4D). Cells were tracked for 13 days (FIG. 2.6F) and confirmed with histology (FIG. 2.7).

Previous work with ultrasound imaging in regenerative medicine has used gold microcapsules⁵⁶ or perfluorcarbon droplets⁵⁷ as ultrasound contrast agents for imaging pancreatic islets. Although microbubble ultrasound contrast agents have a well-detailed mechanism, the nature by which sub-micron materials generate backscatter ultrasound contrast is not immediately clear. B-mode contrast will increase as the impedance mismatch of the sample increases and increasing both the size and number of nanoparticles will increase this mismatch³⁴. We hypothesize that the capacity of the smaller SiNPs to generate US contrast in cells is due to an intracellular aggregation event, which may or may not be due to localization in endosomes. Indeed, sizing of the SiNP fragments present in MSCs by TEM indicated a mean size of 1300 nm versus 300 nm for SiNPs before cell loading. The TEM imaging in FIGS. 2.2, 2 15 and 2.16 illustrate that both large and small SiNPs are present.

This work builds on the long-standing use of MRI in cell tracking and extends it to T1 contrast. The relaxivity of the material at 2.0×10⁶ mM⁻¹ s⁻¹ at 1T or 1.4 mM⁻¹ s⁻¹ on a per Gd³⁺ basis⁵⁸. We suspect that many of the Gd³⁺ ions on the SiNP interior or in the silica framework may not be affecting a T1 shortening. Besides temporal resolution, a fundamental limitation of MRI is poor sensitivity, which means that 50-500 μmol/mL (50 to 500 mM) of iron oxide is needed for T2 imaging³⁰. The SiNPs were detectable in 40 MHz US at 11.5 μg/mL (0.67 pM; molecular weight=1.7×10¹⁰). Furthermore, this system offers a positive contrast mechanism, which is often clinically preferable to negative contrast via iron oxide.

Although the toxicity of free Gd³⁺ is an important concern due to cases of nephrogenic systemic fibrosis (NSF)⁵⁹, less than 5% of Gd³⁺ becomes dissociated after 24 hours of incubation with mouse serum at physiological temperature. If one million cells were implanted the corresponding gadolinium dose would be 9.6×10¹⁵ Gd³⁺ (6540 SiNPs/MSC; 1.47×10⁶ Gd³⁺/SiNP) or 2.5 μg of gadolinium. When we consider that only 5% of the gadolinium becomes dissociated from the SiNP, the effective dose is 0.125 μg gadolinium per 1 million MSCs. From the package insert of the clinically-approved gadolinium-containing agent, Prohance, the recommend dose is 0.1 mmol/kg. For an 82 kg individual, this corresponds to 1.29×10⁶ μg of gadolinium. Thus, the dose encountered by a stem cell therapy patient with these SiNPs is more than 10,000,000-fold lower than a typical T1 enhanced scan with i.v. administration of gadolinium-based contrast. Such a dramatic decrease in overall dose will likely ameliorate any potential risk of NSF. Interestingly, this stability is achieved without the use of chelator or with mesoporous SiNPs; future work will incorporate chelators for even lower dissociation of Gd³⁺.

Analysis of the secretome of the SiNP-labeled MSCs shows a general increase in levels of cytokines. IL-6, IL-8, and MMP-2 are upregulated more than two fold. While some of the cytokines profiled may have both positive and negative effects, many of the broadly implicated positive agents including VEGF, MMP-2, SCF, and MCP-1 are significantly upregulated relative to non-labeled cells. This work is not unexpected—previous work with microarrays has shown that some genetic pathways are upregulated and downregulated after loading stem cells with nanoparticles⁶⁰. Because the MSC mechanism of action is largely considered to be secretome-mediatee^{4,10}, there are large research efforts underway to use physiological preconditioning (hypoxia, serum depletion), genetic manipulation, molecular preconditioning (TNF-α or TGF-α), or pharmacological treatment (Lipopolysaccharides) to increase the expression of these beneficial agents by MSCs⁸. This work with SiNPs is a simple approach that not only allows for cellular imaging, but also increases the output of beneficial components of the MSC secretome.

One arguable limitation of this work is that the contrast agent is diluted during successive generations due to cell division. This is not exclusive to SiNPs and contrast dilution is a common feature of exogenous contrast. Because the signal is intense in both US and MRI mode with low LODs, MSCs can be visualized even with contrast dilution. The initial intracellular concentration was 1 mg/mL. Assuming even distribution of SiNPs between daughter cells, the cells will still have a concentration above the detection limit through generation five (30 μg/mL), which is one generation longer than iron oxide-based approaches. The signal of loaded cells remains significantly (p<0.05) elevated relative to baseline for up to thirteen days, which approximates the 48-hour doubling time we observed in vitro. Furthermore, it is not necessary to label all cells involved in therapy. Because the probe is small and intense, we could label only 5-10% of the cells and use them to track the behavior of the entire cohort. This percentage would still be feasible in clinical applications because millions of cells will be used in humans are used and the 10% that are labeled would still be well within the detection limit of the assay.

Although used tangentially here, fluorescent silica has enjoyed much popularity in the last decade and has even entered clinical trials (NCT01266096)^{28,61}. Red-shifted fluorophores will be used in future studies to facilitate in vivo imaging of the SiNPs via optical imaging in pre-clinical models. We will also study biodegradable SiNPs and SiNPs as a delivery vehicle for a stem cell differentiation agent or prosurvival cocktail^{62,63}. Finally, we will study SiNPs as an alternative to microbubbles for vascular and extravascular imaging of cancer biomarkers in vivo. Importantly, the use of this contrast is not limited to cardiovascular applications. Indeed, ultrasound guided stem cell therapy in most other organ systems would be more straightforward due the high incidence of motion artifacts in the cardiac space. Nevertheless, this work demonstrates the potential utility of SiNP for the treatment of ischemic heart disease with SCT.

Some researchers are developing MRI-compatible catheters for SCT applications¹⁵. Although plausible, such an approach would not have the temporal resolution or cost benefit of US. The approach presented here is useful in bridging the gap between the US and MRI modalities and is a fundamentally new approach to imaging cell implantation. There is currently no alternative technique to allow real time imaging of MSC delivery with such high temporal resolution and high sensitivity. This contrast agent offers a clear, color-coded anatomic interpretation that clearly differentiates between MSCs and the surrounding tissue via standard respiratory and ECG gating procedures, both of which could easily translate into the clinic. In addition, power and color Doppler US can be used in tandem to avoid active blood vessels to reduce blood loss during the graft. Rather than forcing clinicians to choose one modality or the other, this approach satisfies both.

### CONCLUSION

The SiNP detailed above provides contrast for the implantation and monitoring of MSCs. Rather than an implantation event, patient transfer to PET or MRI scanner, and image acquisition, this approach offers instant content on the location and number of implanted cells. The MRI modality has a rich history in cell tracking because of its high resolution and depth insensitive nature, and US has a long-standing role in cardiovascular medicine. Thus, the marriage of US and MRI is ideal for cardiovascular stem cell therapy imaging. This probe is a sub-micron contrast agent, in sharp contrast to the 2-4 μm size of microbubbles, and thus is an exciting alternative to the current paradigm of ultrasound contrast agents because they could potentially leave the vascular space for applications impossible with microbubbles. The probe is quickly endocytosed by MSCs with no transfection reagents, is easy to synthesize reproducibly, and increases the B-mode US and MRI contrast of MSCs by 700% and 200%, respectively, with increased contrast up to thirteen days post-loading and detection limits below 100,000 cells. SiNPs increased the expression level of many of the cytokines implicated in MSC-based SCT. The agent had no significant impact on cell metabolic activity, proliferation, or pluripotency and can likely be used to track ESCs, CPCs, and CSCs. To the best of our knowledge, this is the first application of US-guided MSC therapy and the first report of such a triple modality optical/US/MRI beacon.

References, each of which is incorporated herein by reference
1. Leri, A., Kajstura, J. & Anversa, P. Role of cardiac stem cells in cardiac pathophysiology: a paradigm shift in human myocardial biology. Circ. Res. 109, 941-961 (2011).
2. Domian, I. J., et al. Generation of functional ventricular heart muscle from mouse ventricular progenitor cells. Science 326, 426 (2009).
3. Caspi, O., et al. Tissue engineering of vascularized cardiac muscle from human embryonic stem cells. Circ. Res. 100, 263-272 (2007).
4. Williams, A. R. & Hare, J. M. Mesenchymal stem cells: biology, pathophysiology, translational findings, and therapeutic implications for cardiac disease. Circ. Res. 109, 923-940. (2011).
5. Meyer, G. P., et al. Intracoronary bone marrow cell transfer after myocardial infarction: eighteen months' follow-up data from the randomized, controlled BOOST (BOne marrOw transfer to enhance ST-elevation infarct regeneration) trial. Circulation 113, 1287 (2006).
6. Lee, R. H., et al. Intravenous hMSCs improve myocardial infarction in mice because cells embolized in lung are activated to secrete the anti-inflammatory protein TSG-6. Cell Stem Cell 5, 54-63 (2009).
7. Bolli, R., et al. Cardiac stem cells in patients with ischaemic cardiomyopathy (SCIPIO): initial results of a randomised phase 1 trial. Lancet 378, 1847-1857 (2011).
8. Ranganath, S. H., Levy, O., Inamdar, M. S. & Karp, J. M. Harnessing the mesenchymal stem cell secretome for the treatment of cardiovascular disease. Cell Stem Cell 10, 244-258 (2012).
9. Parekkadan, B. & Milwid, J. M. Mesenchymal stem cells as therapeutics. Annu. Rev. Biomed. Eng. 12, 87-117 (2010).
10. Gnecchi, M., Zhang, Z., Ni, A. & Dzau, V. J. Paracrine mechanisms in adult stem cell signaling and therapy. Circ. Res. 103, 1204-1219 (2008).
11. Hare, J. M., et al. A randomized, double-blind, placebo-controlled, dose-escalation study of intravenous adult human mesenchymal stem cells (prochymal) after acute myocardial infarction. J Am Coll Cardiol 54, 2277-2286 (2009).
12. NIH. www.clinicaltrials.gov. Vol. 2012 (National Institutes of Health, Bethesda, Md., 2011).
13. Nguyen, P. K., Lan, F., Wang, Y. & Wu, J. C. Imaging: guiding the clinical translation of cardiac stem cell therapy. Circ. Res. 109, 962-979. (2011).
14. de Vries, I. J., et al. Magnetic resonance tracking of dendritic cells in melanoma patients for monitoring of cellular therapy. Nat. Biotechnol. 23, 1407-1413. (2005).
15. Bulte, J. W. M. In vivo MRI cell tracking: clinical studies. Am. J. Roentgen. 193, 314 (2009).
16. Kircher, M. F., Gambhir, S. S. & Grimm, J. Noninvasive cell-tracking methods. Nat. Rev. Clin. Pract. 8, 677-688 (2011).
17. Yaghoubi, S. S., et al. Noninvasive detection of therapeutic cytolytic T cells with 18F-FHBG PET in a patient with glioma. Nat. Clin. Pract. Oncol. 6, 53-58 (2009).
18. Adonai, N., et al. Ex vivo cell labeling with 64Cu-pyruvaldehyde-bis(N4-methylthiosemicarbazone) for imaging cell trafficking in mice with positron-emission tomography. Proc. Natl. Acad. Sci. USA 99, 3030 (2002).
19. Modo, M., et al. Tracking transplanted stem cell migration using bifunctional, contrast agent-enhanced, magnetic resonance imaging. Neuroimage 17, 803-811 (2002).
20. Kraitchman, D. L., et al. In vivo magnetic resonance imaging of mesenchymal stem cells in myocardial infarction. Circulation 107, 2290 (2003).
21. Hill, J. M., et al. Serial cardiac magnetic resonance imaging of injected mesenchymal stem cells. Circulation 108, 1009 (2003).
22. Daldrup-Link, H. E., et al. Migration of Iron Oxide-labeled Human Hematopoietic Progenitor Cells in a Mouse Model: In Vivo Monitoring with 1.5-T MR Imaging Equipment. Radiology 234, 197-205 (2005).
23. Shapiro, E. M., Sharer, K., Skrtic, S. & Koretsky, A. P. In vivo detection of single cells by MRI. Magn. Reson. Med. 55, 242-249 (2006).
24. Weissleder, R., et al. Ultrasmall superparamagnetic iron oxide: characterization of a new class of contrast agents for MR imaging. Radiology. 175, 489-493. (1990).
25. Foster Gareau, P., Heyn, C., Alejski, A. & Rutt, B. K. Imaging single mammalian cells with a 1.5 T clinical MRI scanner. Magn. Reson. Med. 49, 968-971 (2003).
26. Nkansah, M. K., Thakral, D. & Shapiro, E. M. Magnetic poly(lactide-co-glycolide) and cellulose particles for MRI-based cell tracking. Magn. Reson. Med. 65, 1776-1785 (2011).
27. Henning, T. D., Boddington, S. & Daldrup-Link, H. E. Labeling hESCs and hMSCs with iron oxide nanoparticles for non-invasive in vivo tracking with MR imaging. JoVE (2008).
28. Hsiao, J. K., et al. Mesoporous silica nanoparticles as a delivery system of gadolinium for effective human stem cell tracking. Small 4, 1445-1452 (2008).
29. Rodriguez-Porcel, M., Gheysens, O., Chen, I. Y., Wu, J. C. & Gambhir, S. S. Image-guided cardiac cell delivery using high-resolution small-animal ultrasound. Mol. Ther. 12, 1142-1147 (2005).
30. Frangioni, J. V. & Hajjar, R. J. In vivo tracking of stem cells for clinical trials in cardiovascular disease. Circulation 110, 3378 (2004).
31. Pysz, M. A. & Willmann, J. K. Targeted contrast-enhanced ultrasound: an emerging technology in abdominal and pelvic imaging. Gastroenterology 140, 785-790. e786.
32. Casciaro, S., et al. Optimal enhancement configuration of silica nanoparticles for ultrasound imaging and automatic detection at conventional diagnostic frequencies. Invest. Radiol. 45, 715.
33. Martinez, H. P., et al. Hard shell gas-filled contrast enhancement particles for colour Doppler ultrasound imaging of tumors. Med. Chem. Commun. 1, 266-270 (2010).
34. Liu, J., et al. Nanoparticles as image enhancing agents for ultrasonography. Phys. Med. Biol. 51, 2179-2189. (2006).
35. Stober, W., Fink, A. & Bohn, E. Controlled growth of monodisperse silica spheres in the micron size range. J. Coll. Interface Sci. 26, 62-69 (1968).
36. Rossi, L. M., Shi, L., Quina, F. H. & Rosenzweig, Z. Stober synthesis of monodispersed luminescent silica nanoparticles for bioanalytical assays. Langmuir 21, 4277-4280 (2005).
37. Westcott, S. L., Oldenburg, S. J., Lee, T. R. & Halas, N. J. Formation and adsorption of clusters of gold nanoparticles onto functionalized silica nanoparticle surfaces. Langmuir 14, 5396-5401 (1998).
38. Dunbar, S. A. Applications of Luminex xMAP™ technology for rapid, high-throughput multiplexed nucleic acid detection. Clin. Chim. Acta. 363, 71-82 (2006).
39. Craig-Schapiro, R., et al. Multiplexed immunoassay panel identifies novel CSF biomarkers for Alzheimer's disease diagnosis and prognosis. PLoS One 6, e18850 (2011).
40. Thakor, A. S., et al. Oxidative Stress Mediates the Effects of Raman Active Gold Nanoparticles in Human Cells. Small 7, 126-136 (2011).
41. AbrÃ moff, M. D., MagalhÃ£es, P. J. & Ram, S. J. Image processing with ImageJ. Biophotonics international 11, 36-42 (2004).
42. Rides, L. M., Nam, S. Y., Sokolov, K., Emelianov, S. Y. & Suggs, L. J. Function of mesenchymal stem cells following loading of gold nanotracers. Int. J. Nanomed. 6, 407-416 (2011).
43. Amaral, M., et al. Si3N4-bioglass composites stimulate the proliferation of MG63 osteoblast-like cells and support the osteogenic differentiation of human bone marrow cells. Biomaterials 23, 4897-4906 (2002).
44. Kostura, L., Kraitchman, D. L., Mackay, A. M., Pittenger, M. F. & Bulte, J. W. Feridex labeling of mesenchymal stem cells inhibits chondrogenesis but not adipogenesis or osteogenesis. NMR Biomed 17, 513-517 (2004).
45. Boddington, S. E., et al. Labeling human mesenchymal stem cells with fluorescent contrast agents: the biological impact. Mol. Imag. Biol. 13, 3-9 (2011).
46. Hackley, V. A. & Clogston, J. D. Measuring the hydrodynamic size of nanoparticles in aqueous media using batch-mode dynamic light scattering. Meth. Mol. Biol. 697, 35 (2011).
47. Xia, T., et al. Polyethyleneimine coating enhances the cellular uptake of mesoporous silica nanoparticles and allows safe delivery of siRNA and DNA constructs. ACS Nano 3, 3273-3286 (2009).
48. Slowing, I. I., Trewyn, B. G., Giri, S. & Lin, V. S. Y. Mesoporous silica nanoparticles for drug delivery and biosensing applications. Adv. Func. Mater. 17, 1225-1236 (2007).
49. Slowing, I., Trewyn, B. G. & Victor, S. Y. L. Effect of surface functionalization of MCM-41-type mesoporous silica nanoparticles on the endocytosis by human cancer cells. J. Am. Chem. Soc 128, 14792-14793 (2006).
50. Chung, T. H., et al. The effect of surface charge on the uptake and biological function of mesoporous silica nanoparticles in 3T3-L1 cells and human mesenchymal stem cells. Biomaterials 28, 2959-2966 (2007).
51. Deshpande, N., Needles, A. & Willmann, J. K. Molecular ultrasound imaging: current status and future directions. Clinical radiology 65, 567-581 (2010).
52. Foster, F., et al. A new ultrasound instrument for in vivo microimaging of mice. Ultrasound Med. Biol. 28, 1165-1172 (2002).
53. Ankrum, J. & Karp, J. M. Mesenchymal stem cell therapy: Two steps forward, one step back. Trends Mol. Med. 16, 203-209.
54. Amado, L. C., et al. Cardiac repair with intramyocardial injection of allogeneic mesenchymal stem cells after myocardial infarction. Proc. Natl. Acad. Sci. USA 102, 11474 (2005).
55. Hare, J. M., et al. A randomized, double-blind, placebo-controlled, dose-escalation study of intravenous adult human mesenchymal stem cells (prochymal) after acute myocardial infarction. J. Am. Coll. Cardiol. 54, 2277-2286 (2009).
56. Arifin, D. R., et al. Trimodal gadolinium-gold microcapsules containing pancreatic islet cells restore normoglycemia in diabetic mice and can be tracked by using US, CT, and positive-contrast MR imaging. Radiology 260, 790-798 (2011).
57. Barnett, B. P., et al. Use of perfluorocarbon nanoparticles for non-invasive multimodal cell tracking of human pancreatic islets. Contrast Media Mol. Imaging 6, 251-259 (2011).
58. Tasciotti, E., et al. Mesoporous silicon particles as a multistage delivery system for imaging and therapeutic applications. Nat. Nanotechnol. 3, 151-157 (2008).
59. Grobner, T. Gadolinium—a specific trigger for the development of nephrogenic fibrosing dermopathy and nephrogenic systemic fibrosis? Neph. Dialy. Transplan. 21, 1104-1108 (2006).
60. Lee, E. S., et al. Microgel iron oxide nanoparticles for tracking human fetal mesenchymal stem cells through magnetic resonance imaging. Stem Cells 27, 1921-1931 (2009).
61. PET Imaging of Patients With Melanoma Using an 1241-labeled cRGDY Silica Nanomolecular Particle Tracer: A Microdosing Study. Vol. NCT01266096 (Memorial Sloan Kettering Cancer Center, clinicaltrials.gov, 2011).
62. Laflamme, M. A., et al. Cardiomyocytes derived from human embryonic stem cells in pro-survival factors enhance function of infarcted rat hearts. Nat. Biotechnol. 25, 1015-1024 (2007).
63. Labbaf, S., et al. Spherical bioactive glass particles and their interaction with human mesenchymal stem cells in vitro. Biomaterials (2010).

### Supplementary Information for Example 3

FIG. 2.8 illustrates the growth curve and spectral behavior of SiNPs. FIG. 2.8A illustrates the size of SiNPs was measured periodically during the synthesis; the maximum size is reached at approximately 2 hours with no further growth seen in overnight incubation. FIG. 2.8B illustrates a UV-Vis absorbance curve of 0.01 mg/mL SiNPs in water. Red arrow indicates FITC absorption. FIG. 2.8C illustrates excitation (solid) and emission (dashed) spectra of SiNPs (1 μg/mL) and stock FITC (1 μM). Spectra are normalized to their maximum. RFU=relative fluorescence intensity. The US signals for different sized SiNPs in an agarose phantom are shown from imaging at 40 MHz (FIG. 2.8D) and 16 (MHz FIG. 2.8E). Error bars represent the standard deviation of five measurements.

FIG. 2.9 illustrates SiNP characterization. FIG. 2.9A illustrates that the SiNP size was determined via TEM images with a mode size of 300 nm. 83% of all NPs were between 200-500 nm. FIG. 2.9B illustrates a histogram of intensity-weighted DLS shows the bias of that technique to larger particles with a Z-average size of 600 nm (0.1 mg/mL in water).

FIG. 2.10 illustrates optimization of US imaging parameters for SiNPs. Imaging conditions were optimized for (FIG. 2.10A) frequency, (FIG. 2.10B) power, (FIG. 2.10C) gain, and (FIG. 2.10D) dynamic range. A 2.5 mg/mL sample of SiNPs was placed in an agarose phantom and imaged at varying input ultrasound frequencies. Signal was defined as B-mode contrast generated from the SiNP inclusion and background was defined as the US contrast generated from the agarose gel surrounding the inclusion. Signal to background ratio (S/B) was calculated as a descriptor of contrast. Error bars represent the standard deviation of five different fields of view. Default settings for this study include 100% power, transducer gain of 20 dB, dynamic range of 60 dB, 40 MHz frequency, and display map G1.

FIG. 2.11 illustrates the concentration-dependent US contrast and ex vivo limit of detection. B-mode ultrasound images of SiNPs in 1% agarose phantom at the following concentrations: FIG. 2.11A, 4.0 mg/mL; FIG. 2.11B, 2.0 mg/mL; FIG. 2.11C, 1.25 mg/mL; FIG. 2.11D, 0.5 mg/mL; FIG. 2.11E, 0.05 mg/mL. FIG. 2.11F is a water control (0 mg/mL SiNPs). Red scale bar in FIG. 2.11A is 2 mm and applies to all images in FIG. 2.11. Intensity scale in A applies to all panels.

FIG. 2.12 illustrates the performance of SiNPs as US contrast ex vivo. FIG. 2.12A illustrates the B-mode ultrasound intensity of SiNPs plotted as a function of concentration shows a linear dynamic range of 0-5 mg/mL at 40 MHz and 0-2.5 mg/mL at 16 MHz. FIG. 2.12B shows lower concentrations and illustrates the LOD of 11.5 and 100.5 μg/mL for 40 and 16 MHz, respectively. Error bars represent standard deviation of five replicate measurements and both are linear at R²>0.99.

FIG. 2.13 illustrates the MRI LOD and T1-Relaxivity of SiNPs. FIG. 2.13A illustrates the decreasing concentrations of SiNPs in water were evaluated ex vivo. (Concentrations in inset: 1-10 mg/mL; 2-5 mg/mL; 3-2.5 mg/mL; 4-1.25 mg/mL; 5-0.625 mg/mL; red box is diluent control) The LOD is 0.03 mg/mL SiNPs. Error bars represent the standard deviation of the three replicate samples. FIG. 2.13B illustrates the T1 of decreasing concentrations of SiNPs from 2.0 to 0.1 mg/mL was measured in a 1T permanent magnet. The inverse of T1 as a function of concentration indicates a T1 relaxivity value of 6×10⁶/(mM s).

FIG. 2.14 illustrates Gd³⁺ loading and stability analyses of SiNPs via ICP. FIG. 2.13A illustrates increasing amounts of SiNPs were analyzed via ICP-AES, with a linear relationship between volume of SiNPs and moles of Gd³⁺ (R²=0.9999). From this, an average of 1.47×10⁶ Gd³⁺ ions per SiNP were calculated. Error bars represent the standard deviation of triplicate samples. FIG. 2.13B illustrates the stability of the SiNP in presence of diluents and murine serum. Less than 5% of the Gd³⁺ dissociated from the SiNP even in serum at 24 hours at body temperature. Error bars represent the standard deviation of triplicate samples.

FIG. 2.15 illustrates the optimization and characterization of SiNP cell loading. FIG. 2.13A), Loading MSCs with increasing amounts of SiNPs indicated that optimal signal (as determined by peak FC signal) was achieved with 0.5 mg/mL SiNPs. FIG. 2.13B), The length of incubation time was similarly optimized and indicated that 6 hours was sufficient. FIG. 2.13C), The stability of cell loading was probed by serially analyzing cells post loading (circles). Intensity reached a ½ max value at approximately two days post loading. Error bars represent the coefficient of variation of the FC histograms for the labeled cells.

FIG. 2.16: Visualizing MSC Loading of SiNPs with Fluorescence Microscopy. Unlabeled MSCs in FIG. 2.16A show little autofluorescence, while labeled MSCs in FIG. 2.16B show green fluorescence from SiNPs via a 10× objective and standard green fluorescent protein optical filters. Green channel images thresholded above 777 on 12 bit images via ImageJ and brightfield images employ 10 ms of exposure time. Scale bar in FIG. 2.16A and FIG. 2.16B is 100 μm. Panels C-E are confocal images under brightfield illumination in FIG. 2.16C, confocal fluorescence through the cells medial slice in FIG. 2.16D, and merged image in FIG. 2.16E. Confocal (Z-axis) scanning indicated that SiNPs were present not only on the cell exterior, but also inside the cell.

FIG. 2.17 illustrates TEM of SiNPs inside MSCs. 500 nm sections of fixed MSCs were imaged with TEM at various magnifications. Sections are coded by color. FIG. 2.17A illustrates 440× magnification shows MSC with dark areas indicating SiNP. FIGS. 2.17B and 2.17C are at 2100× and show both small SiNPs (yellow arrows) and larger clusters of SiNPs (magenta arrows). FIG. 2.17D offers an additional example at 1600×. Size bar in FIG. 2.17A is 5000 nm; FIG. 2.17B and FIG. 2.17C is 1000 nm; FIG. 2.17D is 2000 nm.

FIG. 2.18 illustrates a TEM of SiNPs Inside MSCs at Increasing Magnification. 500 nm sections of fixed MSCs were imaged with TEM at various magnifications. FIGS. 2.18A-C on far left are at 440× magnification. Subset i is at 830×, subset ii is at 1100×, and subset iii is at 2100× (except Aiii at 1600×).

FIG. 2.19 illustrates STEM and EDS mapping of SiNPs inside MSCs. FIGS. 2.19A and 2.19B are STEM images of MSCs including SiNPs (black spots). FIG. 2.19B is magnification of area indicated in FIG. 2.19A. FIG. 2.19C shows a representative positive (red) and negative (black) spectra (with the characteristic silica K electrons at 1700 eV) corresponding to an area with (positive) and without (negative) SiNPs. EDS mapping was done in the areas coded by red and blue boxes, which correspond to FIG. 2.19D and FIG. 2.19E, respectively. FIG. 2.19D and 2.19E show EDS maps of the highlighted areas with the highest red intensity corresponding to most silica. Panel D shows one large (˜500 nm) SiNP and the blue panel (FIG. 2.19E) shows a collection of smaller SiNPs.

FIG. 2.20 illustrates cell-based assays. FIG. 2.20A illustrates the MTT assay was used to count increasing numbers of cells, which confirmed its suitability for cell proliferation assays (FIG. 2.20, main text). Error bars are the standard deviation of four replicate wells and the experiment indicated by asterisk was a positive control that included a cytotoxic agent. FIG. 2.20B illustrates the florescence intensity of the saline wash after cell loading was measured and shows no further decrease after wash 3. Error bars represent the standard deviation of three replicate measurements.

FIG. 2.21 illustrates the cell morphology. MSCs loaded with SiNPs (left) and native cells (right) were studied by light microscopy daily for seven days. Cell morphology is similar in both sets of digital photomicrographs. Images in FIGS. 2.21 A and 2.21B were taken on day 1, FIG. 2.21C and 2.21D are day 3, and FIG. 2.21E and 2.21F day 7.

FIG. 2.22 illustrates a representative T1 weighted image of a phantom with PBS diluent as well as increasing numbers of SiNP-labeled MSCs. Values above section in the phantom indicate the number of MSCs (K=1000; M=million). The * indicates that the 950,000 in that well are unloaded with SiNPs. The presence of the contrast agent increases T1 signal by a factor of 2 (labeled versus unlabeled cells). See FIG. 2.2F.

FIG. 2.23 illustrates the compatibility of SiNP-based contrast with clinical scanners. 40,000 SiNP-loaded MSCs were implanted in an agarose phantom and imaged with 40 MHz (FIG. 2.23A) and 16 MHz (FIG. 2.23B) via the Vevo pre-clinical scanner as well as with a clinical Philips iU22 scanner with 17-5 MHz (FIG. 2.23C) and 12-5 MHz (FIG. 2.23D) transducers. FIG. 2.23E shows replicate measurements as well as the agarose vehicle (diluent). All scale bars are 2 mm and error bars represent standard deviation.

FIG. 2.24 illustrates the impact of SiNP Fraction on US Intensity. FIGS. 2.24A-C are TEM images of a freshly mixed batch of SiNPs showing a distribution of sizes (scale bar=500 nm). The batch was allowed to settle overnight. Then the supernatant was collected and imaged (FIG. 2.24B) showing a more monodisperse collection of SiNPs. The sediment was also imaged and showed larger particles above 1 μm in size. We also measured the US contrast of the three fractions with FIGS. 2.24D-F corresponding to the particle fractions above. Indeed, most of the US signal is generated by the larger aggregates. However, when both the sediment and the supernatant is incubated with cells, the US signal is the same and nearly 7 times higher than unlabeled cells. Error bars represent the standard deviation of five FOVs. Scale bars in FIGS. 2.24A-C are 500 nm. D-F scale bars are 2 mm.

FIG. 2.25 illustrates example Mis-Injection of MSC Detected via US Imaging. In this example, we intended to inject 500,000 SiNP-labeled MSCs (green) into the wall of the LV (red circle), but the bolus was actually delivered in the extra-cardial space. US imaging detected this error in real time.

FIG. 2.26 illustrates the integration of MSCs with LV Wall. FIG. 2.26A illustrates a subset of mouse electrocardiogram after injection of a SiNP-labeled MSC bolus (500,000 MSCs). This section was chosen because it was in between respiratory cycles. FIG. 2.26B illustrates the signal inside an AOI was measured as a function of time (frame rate number). Clear correlation between phase of electrocardiogram and location of the MSC bolus is seen.

FIG. 2.27 illustrates Table S1 Cytokine analysis of MSC secretome. 31 different proteins were measured in the cell culture media of MSCs (column “Normal”) and SiNP-MSCs (column “SiNP”). Culture medium with no MSCs was also analyzed (column “Media”). The limit of detection (LOD) of the assay is presented as well as the coefficient of variation (CV) in the assay at the given concentration regime. Values of p were calculated to determine whether the increase was statistically significant using p=0.05 as the discriminating value.

### Example 3

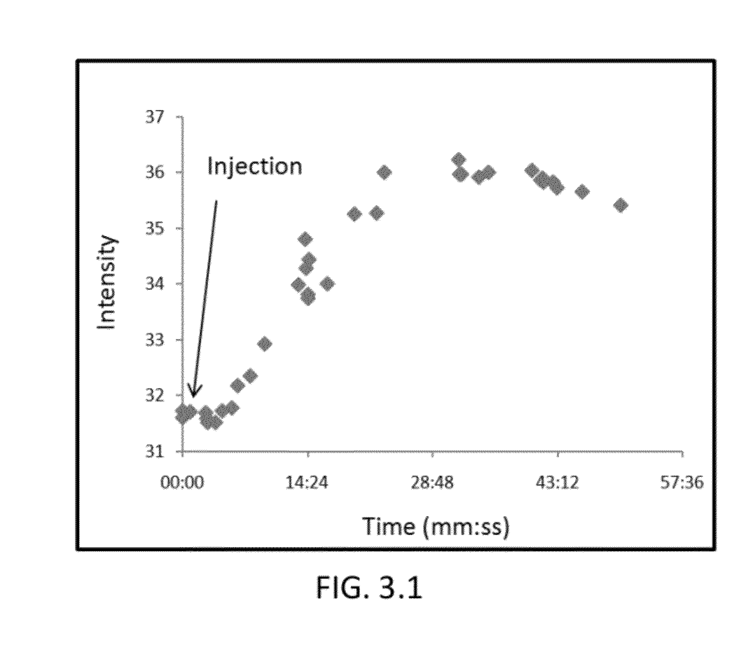

FIG. 3.1 is a graph that illustrates an increase in ultrasound contrast over time after intravenous injection of particles. This figure shows signal increase in a U87MG xenograft tumor after injection of particles. A 12.5% signal increase is realized.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to measurement techniques and the units of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

We claim at least the following:

1. A method of imaging cells, comprising:
disposing one or more cells in a subject, wherein one or more of the cells includes one or more silica nanoparticles within the cell;
imaging the subject and the cells to determine the placement of the cells within the subject; and
detecting an acoustic signal, wherein the acoustic signal correlates to the position of the cells within the subject.

2. The method of claim 1, wherein the imaging is conducted with an ultrasound device.

3. The method of claim 2, further comprising imaging the subject and cells in the B-mode or the contrast mode.

4. The method of claim 1, wherein the silica nanoparticles include a fluorescent agent within the silica nanoparticle.

5. The method of claim 4, further comprising imaging the subject and cells with a fluorescent device, and detecting a fluorescent signal, wherein the fluorescent signal correlates to the position of the cells within the subject.

6. The method of claim 5, wherein the cells are stem cells.

7. The method of claim 1, wherein the silica nanoparticles include a MRI agent.

8. The method of claim 7, further comprising imaging the subject and cells with a MRI device, and detecting a MRI signal, wherein the MRI signal correlates to the position of the cells within the subject.

9. The method of claim 1, wherein the silica nanoparticles include a photoacoustic agent.

10. The method of claim 9, further comprising imaging the subject and cells with a photoacoustic device, and detecting a photoacoustic signal, wherein the photoacoustic signal correlates to the position of the cells within the subject.

11. The method of claim 1, wherein the silica nanoparticles include a radiolabel agent.

12. The method of claim 11, further comprising imaging the subject and cells with a nuclear imaging device, and detecting a nuclear imaging signal, wherein the nuclear imaging signal correlates to the position of the cells within the subject.

13. The method of claim 1, wherein the detecting is done in real-time.

14. A method of making cells, comprising:
exposing one or more cells to a plurality of silica nanoparticles; and
incubating the cells and silica nanoparticles, wherein the silica nanoparticles become disposed within the cells.

15. A method of imaging a target, comprising:
exposing a subject to an imaging device, wherein the subject was introduced to a silica nanoparticle, wherein the silica nanoparticle includes a targeting agent having an affinity for a target; and
detecting the silica nanoparticles, wherein the location of the silica nanoparticles correlates to the location of the target.

16. A method of imaging a target, comprising:
exposing a subject to an imaging device, wherein the subject was introduced to a silica nanoparticle; and
detecting the silica nanoparticles, wherein the location of the silica nanoparticles correlates to the location of the target.

17. A method of imaging and treating a subject, comprising:
administering to a subject in need of treatment a therapeutically effective amount of an agent, wherein the agent is attached to a silica nanoparticle, wherein the silica nanoparticle includes a targeting agent having an affinity for a target;
exposing a subject to an imaging device; and
detecting the silica nanoparticles, wherein the location of the silica nanoparticles correlates to the location of the target.

18. The method of claim 15, further comprising ablating the silica nanoparticles using ultrasonication to release the agents.

19. The method of claim 13, further comprising the detection of the silica nanoparticles in a space decoupled from the vascular space or system circulation.
